(19) United States
(12) Patent Application Publication
(10) Pub. No.: US 2025/0059199 A1
(43) Pub. Date: Feb. 20, 2025

(54) SOLID FORMS OF A TYK2 INHIBITOR, METHOD OF PREPARATION, AND USE THEREOF

(71) Applicant: BeiGene Switzerland GmbH, Basel (CH)

(72) Inventors: Qiuwen Wang, Beijing (CN); Gongyin Shi, Beijing (CN); Yunhang Guo, Beijing (CN); Zhiwei Wang, Beijing (CN); Qian Li, Beijing (CN)

(21) Appl. No.: 18/748,855

(22) Filed: Jun. 20, 2024

Related U.S. Application Data
(63) Continuation of application No. PCT/CN2022/141016, filed on Dec. 22, 2022.

(30) Foreign Application Priority Data
Dec. 23, 2021 (WO) PCT/CN2021/141005
Dec. 2, 2022 (WO) PCT/CN2022/136234

(51) Int. Cl. C07D 491/056; A61K 31/444

(52) U.S. Cl. CPC C07D 491/056; A61K 31/444

(57) ABSTRACT

Disclosed herein are a solid Form of a TYK2 inhibitor N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide per se and the pharmaceutically acceptable salts of the TYK2 inhibitor or crystalline Forms of the salts, pharmaceutical compositions comprising the crystalline Form or the salts or the salts in crystalline Forms, the processes for preparing the crystalline Form or the salts or the salts in crystalline Forms, and methods of use therefor.

### FIELD OF THE DISCLOSURE

Disclosed herein is a solid Form of a TYK2 inhibitor N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide per se and the pharmaceutically acceptable salts of the TYK2 inhibitor or crystalline Forms of the salts. pharmaceutical compositions comprising the crystalline Form or the salts or the salts in crystalline Forms. the processes for preparing the crystalline Form or the salts or the salts in crystalline Forms, and methods of use therefor.

### BACKGROUND OF DISCLOSURE

Janus family of kinases includes JAK1, JAK2, JAK3, and tyrosine kinase 2 (Tyk2) and are nonreceptor tyrosine kinases that bind to the intracellular portion of cell surface cytokine receptors. In response to the stimulation of these receptors. the Janus kinases phosphorylate signal transducer and activator of transcription (STAT) proteins, which then dimerize, translocate to the nucleus, and activate gene transcription. Tyrosine kinase 2 (Tyk2) is a member of the Janus kinase (JAK) family of nonreceptor tyrosine kinases and has been shown to be critical in regulating the signal transduction cascade downstream of receptors for IL-12, IL-23, and type I interferons in both mice (Ishizaki, M. et al., “Involvement of Tyrosine Kinase-2 in Both the IL-12/TH1 and IL-23/TH17 Axes in vivo”, J. Immunol., 187:181-189 (2011); Prchal-Murphyl, M. et al., “TYK2 kinase activity is required for functional type I interferon responses in vivo”, PloS one, 7:e39141 (2012)) and humans (Minegishi, Y. et al., “Human tyrosine kinase 2 deficiency reveals its requisite roles in multiple cytokine signals involved in innate acquired immunity”. Immunity, 25:745-755 (2006)). Tyk2 mediates the receptor-induced phosphorylation of members of the STAT family of transcription factors, an essential signal that leads to the dimerization of STAT proteins and the transcription of STAT-dependent pro-inflammatory genes. Tyk2-deficient mice are resistant to an experimental model of colitis, psoriasis, and multiple sclerosis, demonstrating the importance of Tyk2-mediated signaling in autoimmunity and related disorders (Ishizaki, M. et al., “Involvement of Tyrosine Kinase-2 in Both the IL-12/TH1 and IL-23/TH17 Axes in vivo”, J. Immunol., 187:181-189 (2011); Oyamada, A. et al., “Tyrosine kinase 2 plays critical roles in the pathogenic CD4 T cell responses for the development of experimental autoimmune encephalomyelitis”. J. Immunol., 2009, 183, 7539-7546).

To date, most of the known small molecule JAK inhibitors that have progressed into development are active site-directed inhibitors that bind to the adenosine triphosphate (ATP) site of the catalytic domain (also referred to as the JH1 or “Janus Homology 1” domain) of the JAK protein, which prevents the catalytic activity of the kinase by blocking ATP, downstream phosphorylation, and resulting pathway signal transduction (Bryan, M. et al., “Kinase Inhibitors for the Treatment of Immunological Disorders: Recent Advances”, J. Med. Chem. 2018, 61, 9030-9058). It's well-known that JAK2 is involved in hematopoiesis (Neubauer, H.; et al., “JAK2 deficiency defines an essential developmental checkpoint in definitive hematopoiesis”, Cell 1998, 93, 397-409) and the inhibition of JAK2 can cause side effects such as anemia, neutropeniam and increased infection risk and dyslipidemia (Wollenhaupt, J., et al . . . “Safety and efficacy of tofacitinib, an oral Janus Kinase Inhibitor, for the treatment of rheumatoid arthritis in open-label. J. Rheumatol, 2014, 41, 837-852; He, Y., et al., Efficacy and safety of tofacitinib in the treatment of rheumatoid arthritis: a systematic review and meta-analysis. BMC Musculoskelet. Disord. 2013, 14, 298; Zerbini, C. A, et al., Tofacitinib for the treatment of rheumatoid arthritis. Expert Rev. Clin. Immunol. 2012, 8, 319-331).

N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide (Compound 1) is a TYK2 inhibitor described in an unpublished PCT application No. PCT/CN2021/101282. There is a great need to find solid Forms of Compound 1 or a pharmaceutically acceptable salt thereof, which can provide good stability and good manufacturability suitable for pharmaceutical Formulation. The present disclosure advantageously meets one or more of these requirements.

### SUMMARY OF THE DISCLOSURE

The present disclosure addresses the foregoing challenges and needs by providing a solid Form, particularly a crystalline Form of Compound 1 per se or a pharmaceutically acceptable salt of Compound 1 or the pharmaceutically acceptable salt in crystalline Forms, which are suitable for pharmaceutical use.

The inventors of the present disclosure unexpectedly found 4 crystalline Forms for Compound 1, including (Forms A, B, C, and D), wherein Compound 1 Form A has good physicochemical stability.

The inventors of the present disclosure also found salts of Compound 1, i.e., hydrochloride, sulfate salt, phosphate salt, maleate salt, fumarate salt, HBr salt, mesylate salt, and esilate salt. In particular, the inventors found that the salts of Compound 1 in crystalline Forms, i.e., including crystalline Forms of a hydrochloride salt (Forms B, C, and D), crystalline Forms of a sulfate salt (Forms A and B), a crystalline Form of a phosphate salt Form A, a crystalline Form of a maleate salt From A, crystalline Forms of a fumarate salt (Forms A and B), a crystalline Form of an HBr salt Form A, crystalline Forms of a mesylate salt (Forms A, B, and C), and crystalline Forms of an esilate salt (Forms A and B).

Among the crystalline Forms of Compound 1, Compound 1 mesylate Form C, phosphate Form A, malcate Form A, fumarate Form B, and hydrochloride Form C and hydrochloride Form D are physically and chemically stable under 25° C./60% RH or 40° C./75% RH over one week. And, some Forms are physically and chemically stable under 25° C./60% RH, 30° C./65% RH, or 40° C./75% RH in a tight container over six months.

Compound 1 mesylate Form C, phosphate Form A, maleate Form A and hydrochloride Form C are slightly hygroscopic, and Compound 1 hydrochloride Form D is non-hygroscopic. In addition, the crystalline Forms, including Compound 1 mesylate From C, phosphate Form A, maleate Form A, hydrochloride Form C, and hydrochloride Form D show good tolerance to compression, dry grinding, granulation simulation with no Form change, and no obvious crystallinity decrease, and Compound 1 hydrochloride Form C showed good tolerance to compression with no Form change and no obvious crystallinity decrease, but the XRPD peaks became slightly broadened when compressed at 5 MPa, 10 MPa, and 20 MPa.

In addition, some crystalline Forms, e.g., Compound 1 mesylate Form C, phosphate Form A, maleate Form A, or fumarate Form B show relatively high solubility in some solvent systems as well as good PK exposure.

In a first aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide, said Form is designated as Compound 1 Form A (i.e., the crystalline Form of Compound 1 per se).

In some embodiments, Compound 1 Form A has an X-ray powder diffraction pattern comprising diffraction peaks having °2θ angle values at 10.3±0.2°, 14.8±0.2°, and 16.2°±0.2°.

In some embodiments, Compound 1 Form A has an X-ray powder diffraction pattern comprising diffraction peaks having °2θ angle values at 10.3±0.2°, 14.8±0.2°, 16.2±0.2°, 20.7±0.2° and 21.6±0.2°.

In some embodiments, Compound 1 Form A has an X-ray powder diffraction pattern comprising diffraction peaks having °2θ angle values at 7.5±0.2°, 10.3±0.2°, 14.8±0.2°, 16.2±0.2°, 20.7±0.2°, and 21.6±0.2°.

In some embodiments, Compound 1 Form A has an X-ray powder diffraction pattern comprising diffraction peaks having °2θ angle values at 7.5±0.2°, 10.3±0.2°, 14.8±0.2°, 15.4±0.2°, 16.2±0.2°, 19.8±0.2°, 20.7±0.2°, 21.6±0.2°, 22.8±0.2° and 25.0±0.2°.

In some embodiments, Compound 1 Form A has an XRPD pattern substantially as shown in FIG. 1A.

In some embodiments, Compound 1 Form A is characterized by having one endotherm peak at about 305° C. by differential scanning calorimetry (DSC).

In some embodiments, Compound 1 Form A has a DSC thermogram substantially as shown in FIG. 1D.

In some embodiments, Compound 1 Form A is an anhydrate.

In a second aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro- 2H-pyran-4-yl)pyridin-2-yl)amino)pyridin-2-yl)acetamide maleate, said Form is designated as Compound 1 maleate Form A.

In some embodiments, Compound 1 maleate Form A has an X-ray powder diffraction pattern comprising diffraction peaks having °2θ angle values at 11.8±0.2°, 16.4±0.2° and 21.8±0.2°.

In some embodiments, Compound 1 maleate Form A has an X-ray powder diffraction pattern comprising diffraction peaks having °2θ angle values at 5.6=0.2°, 11.8±0.2°, 16.4±0.2°, 18.2±0.2°, 20.8±0.2° and 21.8±0.2°.

In some embodiments, Compound 1 maleate Form A has an X-ray powder diffraction pattern comprising diffraction peaks having °2θ angle values at 5.6±0.2°, 11.8±0.2°, 16.4±0.2°, 18.2±0.2°, 19.1±0.2°, 20.8±0.2°, 21.8±0.2° and 23.9±0.2°.

In some embodiments, Compound 1 maleate Form A has an X-ray powder diffraction pattern comprising diffraction peaks having °2θ angle values at 5.6±0.2°, 11.8±0.2°, 16.4±0.2°, 17.4±0.2°, 18.2±0.2°, 19.1±0.2°, 20.8±0.2°, 21.8±0.2° and 23.9±0.2°.

In some embodiments, Compound 1 maleate Form A has an XRPD pattern substantially as shown in FIG. 5A.

In some embodiments, Compound 1 maleate Form A is characterized by having one endotherm peak at about 215° C. by differential scanning calorimetry (DSC).

In some embodiments, Compound 1 maleate Form A has a DSC thermogram substantially as shown in FIG. 5D.

In some embodiments, Compound 1 maleate Form A is an anhydrate.

In a third aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide phosphate, said Form is designated as Compound 1 phosphate Form A.

In some embodiments, Compound 1 phosphate Form A has an X-ray powder diffraction pattern comprising diffraction peaks having °2θ angle values at 11.1±0.2°, 12.6±0.2°, and 19.8±0.2°.

In some embodiments, Compound 1 phosphate Form A has an X-ray powder diffraction pattern comprising diffraction peaks having °2θ angle values at 7.0±0.2°, 11.1±0.2°, 12.6±0.2°, 14.5±0.2° and 19.8±0.2°.

In some embodiments, Compound 1 phosphate Form A has an X-ray powder diffraction pattern comprising diffraction peaks having °2θ angle values at 7.0±0.2°, 11.1±0.2°, 12.6±0.2°, 14.5±0.2°, 18.1±0.2°, 19.8±0.2° and 21.7±0.2°.

In some embodiments, Compound 1 phosphate Form A has an X-ray powder diffraction pattern comprising diffraction peaks having °2θ angle values at 7.0±0.2°, 11.1±0.2°, 12.6±0.2°, 14.5±0.2°, 18.1±0.2°, 19.8±0.2°, 21.7±0.2°, 22.9±0.2° and 24.1±0.2°.

In some embodiments, Compound 1 phosphate Form A has an XRPD pattern substantially as shown in FIG. 4A.

In some embodiments, Compound 1 phosphate Form A is characterized by having one decomposition upon melting peak having an onset of 234° C. by differential scanning calorimetry (DSC).

In some embodiments, Compound 1 phosphate Form A has a DSC thermogram substantially as shown in FIG. 4C.

In some embodiments, Compound 1 phosphate Form A is an anhydrate.

In a forth aspect, disclosed herein is a crystalline Form of N-(5-(2,2-dimethyl-2,3-dihydro-[1,4]dioxino[2.3-b]pyridin-6-yl)-4-((6-(methylsulfonyl)-4-(tetrahydro-2H-pyran-4-yl)pyridin-2-yl)amino)pyridin-2-yl)acetamide hydrochloride, said Form is designated as Compound 1 hydrochloride Form C.

In some embodiments, Compound 1 hydrochloride Form C has an X-ray powder diffraction pattern comprising diffraction peaks having °2θ angle values at 9.1±0.2°, 13.8±0.2° and 21.3±0.2°.

In some embodiments, Compound 1 hydrochloride Form C has an X-ray powder diffraction pattern comprising diffraction peaks having °2θ angle values at 9.1±0.2°, 13.8±0.2°, 18.1±0.2° and 21.3±0.2°.

In some embodiments, Compound 1 hydrochloride Form C has an X-ray powder diffraction pattern comprising diffraction peaks having °2θ angle values at 9.1±0.2°, 13.8±0.2°, 18.1±0.2°, 19.9±0.2°, 21.3±0.2° and 24.0+0.2°.

In some embodiments, Compound 1 hydrochloride Form C has an X-ray powder diffraction pattern comprising diffraction peaks having °2θ angle values at 6.9±0.2°, 9.1±0.2°, 13.8±0.2°, 18.1±0.2°, 19.9±0.2°, 21.3±0.2° and 24.0±0.2°.

In some embodiments, Compound 1 hydrochloride Form C has an X-ray powder diffraction pattern comprising diffraction peaks having °2θ angle values at 6.9±0.2°, 9.1±0.2°, 13.8±0.2°, 18.1±0.2°, 19.9±0.2°, 21.3±0.2° and 24.0±0.2°.

In some embodiments, Compound 1 hydrochloride Form C has an X-ray powder diffraction pattern comprising diffraction peaks having °2θ angle values at 6.9±0.2°, 9.1±0.2°, 13.8±0.2°, 16.2±0.2°, 18.1±0.2°,19.2±0.2°, 19.9±0.2°, 21.3±0.2°, 24.0±0.2°, and 24.3±0.2°.

In some embodiments, the crystal system of Compound 1 hydrochloride Form C is monoclinic and the space group is P2₁/c having the cell parameters: (a) is about 9.846 (5), (b) is about 25.660 (11) Å, (c) is about 10.975 (4) Å, and (β) is about 92.551 (15)°.

In some embodiments, Compound 1 hydrochloride Form C has an XRPD pattern substantially as shown in FIG. 2G.

In some embodiments, Compound 1 hydrochloride Form C has a DSC thermogram substantially as shown in FIG. 2I.

In a fifth aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide hydrochloride, said Form is designated as Compound 1 hydrochloride Form D.

In some embodiments, Compound 1 hydrochloride Form D has an X-ray powder diffraction pattern comprising diffraction peaks having °2θ angle values at 9.9±0.2° and 20.4±0.2°.

In some embodiments, Compound 1 hydrochloride Form D has an X-ray powder diffraction pattern comprising diffraction peaks having °2θ angle values at 7.2±0.2°, 9.9±0.2° and 20.4±0.2°.

In some embodiments, Compound 1 hydrochloride Form D has an X-ray powder diffraction pattern comprising diffraction peaks having °2θ angle values at 7.2±0.2°, 9.9±0.2°, 14.4±0.2°, 20.4±0.2° and 23.5±0.2°.

In some embodiments, Compound 1 hydrochloride Form D has an X-ray powder diffraction pattern comprising diffraction peaks having °2θ angle values at 7.2±0.2°, 9.9±0.2°, 14.4±0.2°, 17.5±0.2°, 20.4±0.2°, 21.6±0.2°, and 23.5±0.2°.

In some embodiments, Compound 1 hydrochloride Form D has an X-ray powder diffraction pattern comprising diffraction peaks having °2θ angle values at 7.2±0.2°, 9.9±0.2°, 13.2±0.2°, 14.4±0.2°, 17.5±0.2°, 19.1±0.2°, 20.4±0.2°, 21.6±0.2° and 23.5±0.2°.

In some embodiments, Compound 1 hydrochloride Form D has an X-ray powder diffraction pattern comprising diffraction peaks having °2θ angle values at 7.2±0.2°, 9.9±0.2°, 13.2±0.2°, 14.4±0.2°, 16.5±0.2°, 17.5±0.2°, 19.1±0.2°, 20.4±0.2°, 21.6±0.2°, 23.5±0.2° and 24.9±0.2°.

In some embodiments, the crystal system of Compound 1 hydrochloride Form D is monoclinic and the space group is P2₁/n having the cell parameters: (a) is about 10.309 Å, (b) is about 10.855 (7) Å, (c) is about 24.995 (17), and (β) is about 98.84 (3)°.

In some embodiments, Compound 1 hydrochloride Form D has an XRPD pattern substantially as shown in FIG. 2K.

In some embodiments, Compound 1 hydrochloride Form D is characterized by having one endotherm peak at about 176° C. by differential scanning calorimetry (DSC).

In some embodiments, Compound 1 hydrochloride Form D has a DSC thermogram substantially as shown in FIG. 2M.

In a sixth aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide fumarate, said Form is designated as Compound 1 fumarate Form B.

In some embodiments, Compound 1 fumarate Form B has an X-ray powder diffraction pattern comprising diffraction peaks having °2θ angle values at 12.2±0.2°, 15.5±0.2°, and 20.4±0.2°.

In some embodiments, Compound 1 fumarate Form B has an X-ray powder diffraction pattern comprising diffraction peaks having °2θ angle values at 6.9±0.2°, 12.2±0.2°, 15.5±0.2°, 17.2±0.2°, and 20.4±0.2°.

In some embodiments, Compound 1 fumarate Form B has an X-ray powder diffraction pattern comprising diffraction peaks having °2θ angle values at 6.9±0.2°, 12.2±0.2°, 15.5±0.2°, 17.2±0.2°, 19.6±0.2°, 20.4±0.2°, and 21.8±0.2°.

In some embodiments, Compound 1 fumarate Form B has an X-ray powder diffraction pattern comprising diffraction peaks having °2θ angle values at 6.9±0.2°, 12.2±0.2°, 15.5±0.2°, 17.2±0.2°, 19.6±0.2°, 20.4±0.2°, 21.8±0.2°, and 23.3±0.2°.

In some embodiments, Compound 1 fumarate Form B has an XRPD pattern substantially as shown in FIG. 6E.

In some embodiments, Compound 1 fumarate Form B is characterized by having one endotherm peak (a melting peak) at about 235° C. by differential scanning calorimetry (DSC).

In some embodiments, Compound 1 fumarate Form B has a DSC thermogram substantially as shown in FIG. 6G.

In some embodiments, Compound 1 fumarate Form B is an anhdyrate.

In a seventh aspect, disclosed herein is a crystalline Form of N-(5-(2,2-dimethyl-2,3-dihydro-[1.4]dioxino[2,3-b]pyridin-6-yl)-4-((6-(methylsulfonyl)-4-(tetrahydro- 2H-pyran-4-yl)pyridin-2-yl)amino)pyridin-2-yl)acetamide mesylate, said Form is designated as Compound 1 mesylate Form C.

In some embodiments Compound 1 mesylate Form C.has an X-ray powder diffraction pattern comprising diffraction peaks having °2θ angle values at 6.6±0.2°, 13.3±0.2°, and 15.4±0.2°.

In some embodiments, Compound 1 mesylate Form C.has an X-ray powder diffraction pattern comprising diffraction peaks having °2θ angle values at 6.6±0.2°, 13.3±0.2°, 15.4±0.2°, 16.6±0.2°, and 21.6±0.2°.

In some embodiments, Compound 1 mesylate Form C.has an X-ray powder diffraction pattern comprising diffraction peaks having °2θ angle values at 6.6±0.2°, 13.3±0.2°, 15.4±0.2°, 16.6±0.2°, 21.6±0.2°, and 23.0±0.2°.

In some embodiments, Compound 1 mesylate Form C.has an X-ray powder diffraction pattern comprising diffraction peaks having °2θ angle values at 6.6±0.2°, 13.3±0.2°, 15.4±0.2°, 16.6±0.2°, 21.6±0.2°, and 23.0±0.2°.

In some embodiments, Compound 1 mesylate Form C.has an X-ray powder diffraction pattern comprising diffraction peaks having °2θ angle values at 6.6±0.2°, 7.7±0.2°, 13.3±0.2°, 15.4±0.2°, 16.6±0.2°, 19.9±0.2°, 21.6±0.2°, and 23.0±0.2°.

In some embodiments, Compound 1 mesylate Form C has an XRPD pattern substantially as shown in FIG. 8I.

In some embodiments, Compound 1 mesylate Form C is characterized by having one endotherm peak at about 122° C. by differential scanning calorimetry (DSC).

In some embodiments, Compound 1 mesylate Form C has a DSC thermogram substantially as shown in FIG. 8K.

In an eighth aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro- 2H-pyran-4-yl)pyridin-2-yl)amino)pyridin-2-yl)acetamide, said Form is designated as Compound 1 Form E.

In some embodiments, Compound 1 Form E has an X-ray powder diffraction pattern comprising diffraction peaks having °2θ angle values at 4.3±0.2°, 6.9±0.2°, and 18.4°±0.2°.

In some embodiments, Compound 1 Form E has an X-ray powder diffraction pattern comprising diffraction peaks having °2θ angle values at 4.3±0.2°, 6.9±0.2°, 13.1°±0.2, 13.7°±0.2 and 18.4±0.2°.

In some embodiments, Compound 1 Form E has an X-ray powder diffraction pattern comprising diffraction peaks having °2θ angle values at 4.3±0.2°, 6.9±0.2°, 13.1°±0.2, 13.7°±0.2, 18.4±0.2° and 19.6°±0.2,.

In some embodiments, Compound 1 Form E has an X-ray powder diffraction pattern comprising diffraction peaks having °2θ angle values at 4.3±0.2°, 6.9±0.2°, 13.1°±0.2, 13.7°±0.2, 16.5±0.2°, 18.4±0.2°, 19.6±0.2°, 21.7°±0.2 and 24.7±0.2°.

In some embodiments, Compound 1 Form E has an XRPD pattern substantially as shown in FIG. 10A.

In some embodiments, Compound 1 Form E is characterized by having two endotherm peaks at about 81° C. and about 303° C., and having one exotherm peak at about 148° C. by differential scanning calorimetry (DSC).

In some embodiments, Compound 1 Form E has a DSC thermogram substantially as shown in FIG. 10B.

In a ninth aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide, said Form is designated as Compound 1 Form B.

In some embodiments, Compound 1 Form B has an X-ray powder diffraction pattern comprising diffraction peaks having °2θ angle values at 6.4±0.2°, 7.4°±0.2° and 13.2±0.2°.

In some embodiments, Compound 1 Form B has an X-ray powder diffraction pattern comprising diffraction peaks having °2θ angle values at 4.9±0.2°, 6.4±0.2°, 7.4°±0.2°, 13.2±0.2° and 17.7±0.2°.

In some embodiments, Compound 1 Form B has an X-ray powder diffraction pattern comprising diffraction peaks having °2θ angle values at 4.9±0.2°, 6.4±0.2°, 7.4°±0.2, 9.8°±0.2, 13.2±0.2° and 17.7±0.2°.

In some embodiments, Compound 1 Form B has an X-ray powder diffraction pattern comprising diffraction peaks having °2θ angle values at 4.9±0.2°, 6.4±0.2°, 7.4°±0.2, 9.8°±0.2, 12.7±0.2°, 13.2±0.2°, 16.3±0.2°, 17.7±0.2°, and 18.1±0.2°.

In some embodiments, Compound 1 Form B has an XRPD pattern substantially as shown in FIG. 1D.

In some embodiments, Compound 1 Form B is characterized by having four endotherm peaks at about 75° C. about 123° C. about 174° C. and about 301° C. by differential scanning calorimetry (DSC).

In some embodiments, Compound 1 Form B has a DSC thermogram substantially as shown in FIG. 1F.

In some embodiments, Compound 1 Form B is a TFE and water hetero-solvate.

In a tenth aspect, disclosed herein is a crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide, said Form is designated as Compound 1 Form C.

In some embodiments, Compound 1 Form C has an X-ray powder diffraction pattern comprising diffraction peaks having °2θ angle values at 14.8±0.2°, 17.7±0.2°, and 20.7°±0.2°.

In some embodiments, Compound 1 Form C has an X-ray powder diffraction pattern comprising diffraction peaks having °2θ angle values at 14.8±0.2°, 17.4±0.2°, 17.7°±0.2, 20.7±0.2° and 21.6±0.2°.

In some embodiments, Compound 1 Form C has an X-ray powder diffraction pattern comprising diffraction peaks having °2θ angle values at 10.8±0.2°, 14.8±0.2°, 17.4°±0.2, 17.7±0.2°, 20.7±0.2°, and 21.6±0.2°.

In some embodiments, Compound 1 Form C has an X-ray powder diffraction pattern comprising diff80raction peaks having °2θ angle values at 10.8±0.2°, 14.4±0.2°, 14.8°±0.2, 15.3±0.2°, 15.7±0.2°, 17.4±0.2°, 17.7±0.2°, 20.7±0.2°, and 21.6±0.2°.

In some embodiments, Compound 1 Form C has an XRPD pattern substantially as shown in FIG. 1H.

In some embodiments, Compound 1 Form C is characterized by having an endothermic peak at about 86° C. (Tonset), followed by an endothermic peak at about 138° C. and a melting peak at about 303° C. by differential scanning calorimetry (DSC).

In some embodiments, Compound 1 Form C has a DSC thermogram substantially as shown in FIG. 1J.

In some embodiments, Compound 1 Form C is a 1,4-dioxane solvate.

In an eleventh aspect, disclosed herein is a crystalline Form of N-(5-(2,2-dimethyl-2,3-dihydro-[1,4]dioxino[2,3-b]pyridin-6-yl)-4-((6-(methylsulfonyl)-4- (tetrahydro-2H-pyran-4-yl)pyridin-2-yl)amino)pyridin-2-yl)acetamide, said Form is designated as Compound 1 Form D.

In some embodiments, Compound 1 Form D has an X-ray powder diffraction pattern comprising diffraction peaks having °2θ angle values at 16.1±0.2°, 16.5±0.2°, and 18.8°±0.2°.

In some embodiments, Compound 1 Form D has an X-ray powder diffraction pattern comprising diffraction peaks having °2θ angle values at 9.8±0.2°, 16.1±0.2°, 16.5°±0.2°, 18.1°±0.2°, and 18.8°±0.2°.

In some embodiments, Compound 1 Form D has an X-ray powder diffraction pattern comprising diffraction peaks having °2θ angle values at 9.4±0.2°, 9.8±0.2°, 16.1°±0.2°, 16.5°±0.2°, 18.1±0.2°, and 18.8±0.2°.

In some embodiments, Compound 1 Form D has an X-ray powder diffraction pattern comprising diffraction peaks having °2θ angle values at 9.4±0.2°, 9.8±0.2°, 16.1°±0.2°, 16.5°±0.2°, 18.1±0.2°, 18.8±0.2°, 21.6±0.2°, 22.7±0.2°, and 24.2±0.2°.

In some embodiments, Compound 1 Form D has an XRPD pattern substantially as shown in FIG. 1L.

In some embodiments, Compound 1 Form D is characterized by having an endothermic peak at about 105° C. (Tonset), followed by a melting peak at about 303° C. by differential scanning calorimetry (DSC).

In some embodiments, Compound 1 Form D has a DSC thermogram substantially as shown in FIG. 1N.

In some embodiments, Compound 1 Form D is a chloroform solvate.

In some embodiments of all above aspects, the crystalline Forms have at least 40%, 50%, 60%, 70%, 80%, 90% or 95% crystallinity. In some embodiments of all above aspects, the crystalline Forms are at least 40%, 50%, 60%, 70%, 80%, 90% or 95% crystalline.

In a twelfth aspect, disclosed herein is a pharmaceutical composition, comprising a therapeutically effective amount of a crystalline Form of Compound 1 or a crystalline Form of a pharmaceutically acceptable salt of Compound 1 according to any one of the above aspects, and at least one pharmaceutically acceptable excipient.

In a thirteenth aspect, disclosed herein is a method for treating an inflammatory or autoimmune disease in a subject in a subject in need thereof comprising administering to the subject a therapeutically effective amount of a crystalline Form of Compound 1 or a crystalline Form of a pharmaceutically acceptable salt of Compound 1 according to any one of the above aspects. In some embodiments, the subject is a human.

In some embodiments, disclosed herein is a process for preparing Compound 1 Form A comprising
a) dissolving Compound 1 in N-methylpyrrolidone or Dimethylacetamide, adding acetone or butanone, cooling, to obtain Compound 1 Form A.

In some embodiments, disclosed herein is a process for preparing Compound 1 Form A comprising any one of the following procedures:
a) dissolving Compound 1 in N-methylpyrrolidone, adding acetone, cooling, to obtain Compound 1 Form A; or
b) dissolving Compound 1 in Dimethylacetamide, adding butanone, cooling, to obtain Compound 1Form A.

In some embodiments, disclosed herein is a process for preparing Compound 1 maleate Form A comprising:
a) adding Compound 1 or Compound 1 Form A and maleic acid into acetone, stirring, cooling, to obtain Compound 1 maleate Form A.

In some embodiments, disclosed herein is a process for preparing Compound 1 phosphate Form A comprising:
a) dissolving Compound 1 or Compound 1 Form A in ACN (acetonitrile), adding phosphoric acid/ACN solution, stirring, to obtain Compound 1 phosphate Form A.

In some embodiments, disclosed herein is a process for preparing Compound 1 hydrochloride Form C comprising any one of the following procedures:
a) dissolving Compound 1 or Compound 1 Form A in MeOH/H₂O (v/v=9/1), adding HCl, stirring, cooling, to obtain Compound 1 hydrochloride Form C;
b) dissolving Compound 1 or Compound 1 Form A in MeOH, adding HCl/MeOH solution, stirring, cooling, to obtain Compound 1 hydrochloride Form C;
c) dissolving Compound 1 or Compound 1 Form A in NMP/MeOH (v/v=1/4), adding HCl/MeOH solution, stirring, cooling, to obtain Compound 1 hydrochloride Form C; or
d) dissolving Compound 1 or Compound 1 Form A in water, heating, adding HCl, stirring, adding acetone, cooling, to obtain the Compound 1 hydrochloride Form C.

In some embodiments, disclosed herein is a process for preparing Compound 1 hydrochloride Form C comprising any one of the following procedures:
a) dissolving Compound 1 or Compound 1 Form A in MeOH/H₂O (v/v=9/1), adding HCl, stirring below about 50° C., cooling, to obtain Compound 1 hydrochloride Form C;
b) dissolving Compound 1 or Compound 1 Form A in MeOH, heating to about 60° C., adding HCl/MeOH solution, stirring, cooling, to obtain Compound 1 hydrochloride Form C;
c) dissolving Compound 1 or Compound 1 Form A in NMP/MeOH (v/v=1/4), adding HCl/MeOH solution, stirring under about 50° C., cooling, to obtain Compound 1 hydrochloride Form C; or
d) dissolving Compound 1 or Compound 1 Form A in water, heating to about 50° C., adding HCl, stirring, adding acetone, cooling, to obtain the Compound 1 hydrochloride Form C.

In some embodiments, disclosed herein is a process for preparing Compound 1 hydrochloride Form D comprising:
a) equilibrating Compound 1 hydrochloride Pattern A in a solvent/water system and ACN/water (90/10, v/v), to obtain Compound 1 hydrochloride Form D.

In some examples of these embodiments, the solvent/water system is acetone/water (90/10,v/v) and THF/water (85/15,v/v); or ACN/water (90/10,v/v) or acetone/water (80/20,v/v) and MEK/water (95/15,v/v); or ACN/water (90/10,v/v). More specifically, Compound 1 hydrochloride Form D is obtained from acetone/water (90/10,v/v) and THF/water (85/15,v/v) by equilibration at 25° C. for 24 days; from ACN/water (90/10,v/v) by equilibration at 25° C. for 14 days; from acetone/water (80/20,v/v) and MEK/water (95/15,v/v) by equilibration at 25° C. for 76 days; from ACN/water (90/10,v/v) by equilibration at 50° C. for 7 days.

In some embodiments, disclosed herein is a process for preparing Compound 1 hydrochloride Form D comprising:
a) equilibrating Compound 1 hydrochloride Pattern A in a solvent/water system at about 23° C. and ACN/water (90/10, v/v) at 50° C. to obtain Compound 1 hydrochloride Form D.

In some examples of these embodiments, the solvent/water system is acetone/water (90/10,v/v) and THF/water (85/15,v/v); or ACN/water (90/10,v/v) or acetone/water (80/20,v/v) and MEK/water (95/15,v/v); or ACN/water (90/10,v/v). More specifically, Compound 1 hydrochloride Form D is obtained from acetone/water (90/10,v/v) and THF/water (85/15,v/v) by equilibration at 25° C. for 24 days; from ACN/water (90/10,v/v) by equilibration at 25° C. for 14 days; from acetone/water (80/20,v/v) and MEK/water (95/15,v/v) by equilibration at 25° C. for 76 days; from ACN/water (90/10,v/v) by equilibration at 50° C. for 7 days.

In some embodiments, disclosed herein is a process for preparing Compound 1 fumarate Form B comprising any one of the following procedures:
a) adding Compound 1 or Compound 1 Form A and fumaric acid into acetone, stirring, to obtain Compound 1 fumarate Form B; or
b) dissolving Compound 1 or Compound 1 Form A in ACN, adding fumaric acid, stirring, to obtain Compound 1 fumarate Form B.

In some embodiments, disclosed herein is a process for preparing Compound 1 mesylate Form C comprising:
a) dissolving Compound 1 or Compound 1 Form A in acetone/H₂O (v/v=9/1), adding methane sulfonic acid, stirring, to obtain Compound 1 mesylate Form C.

In some embodiments, any process as described above further comprises adding a crystal seed in the solution system.

### BRIEF DESCRIPTION OF THE FIGURES

FIG. 1A illustrates an X-ray powder diffraction (XRPD) pattern of Compound 1 Form A (anhydrate).

FIG. 1B illustrates a differential scanning calorimetry (DSC) profile/thermogravimetric analysis (TGA) profile of Compound 1 Form A.

FIG. 1C illustrates a ¹H-nuclear magnetic resonance (¹H-NMR) spectrum of Compound 1 Form A.

FIG. 1D illustrates an X-ray powder diffraction (XRPD) pattern of Compound 1 Form B (TFE and water hetero-solvate).

FIG. 1E illustrates a thermogravimetric analysis (TGA) profile of Compound 1 Form B (TFE and water hetero-solvate).

FIG. 1F illustrates a differential scanning calorimetry (DSC) profile of Compound 1 Form B (TFE and water hetero-solvate).

FIG. 1G illustrates a ¹H-nuclear magnetic resonance (¹H-NMR) spectrum of Compound 1 Form B (TFE and water hetero-solvate).

FIG. 1H illustrates an X-ray powder diffraction (XRPD) pattern of Compound 1 Form C (1,4-dioxane solvate).

FIG. 11 illustrates a thermogravimetric analysis (TGA) profile of Compound 1 Form C (1,4-dioxane solvate).

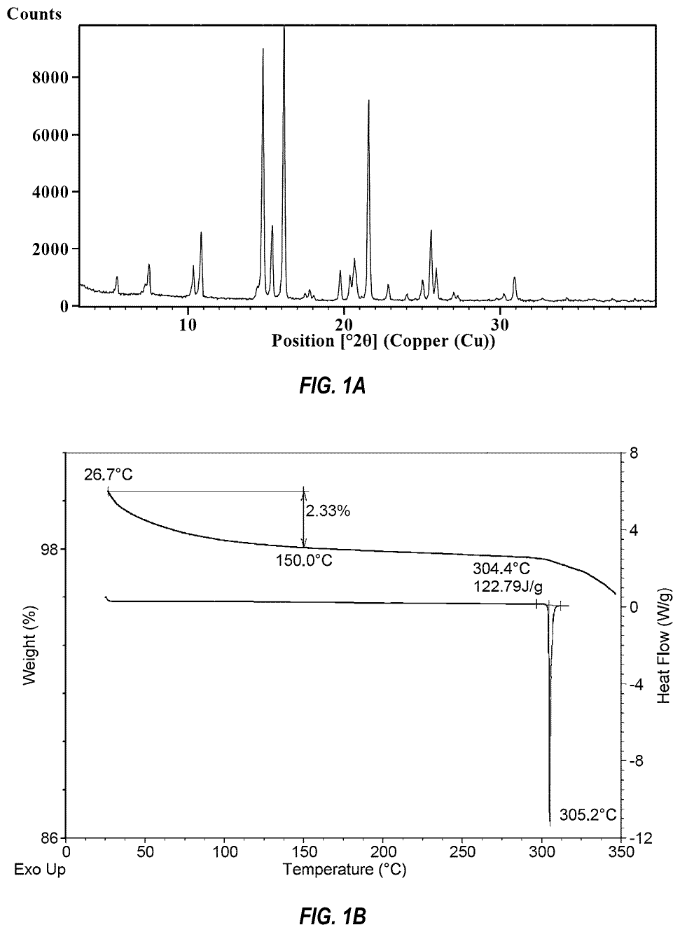

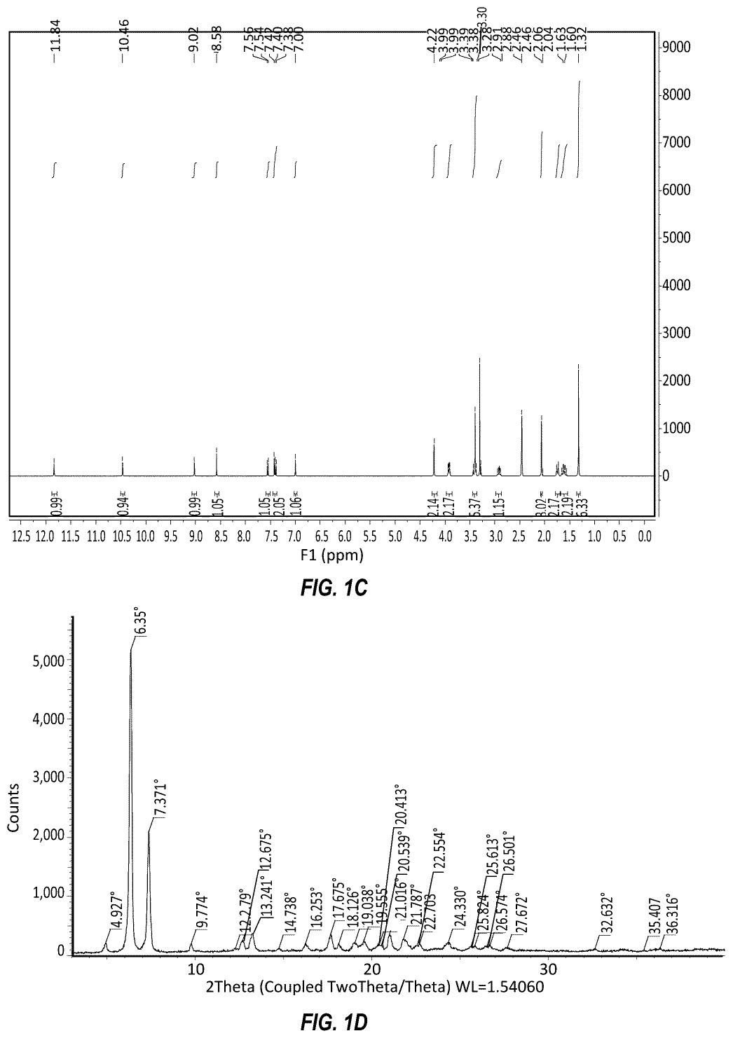

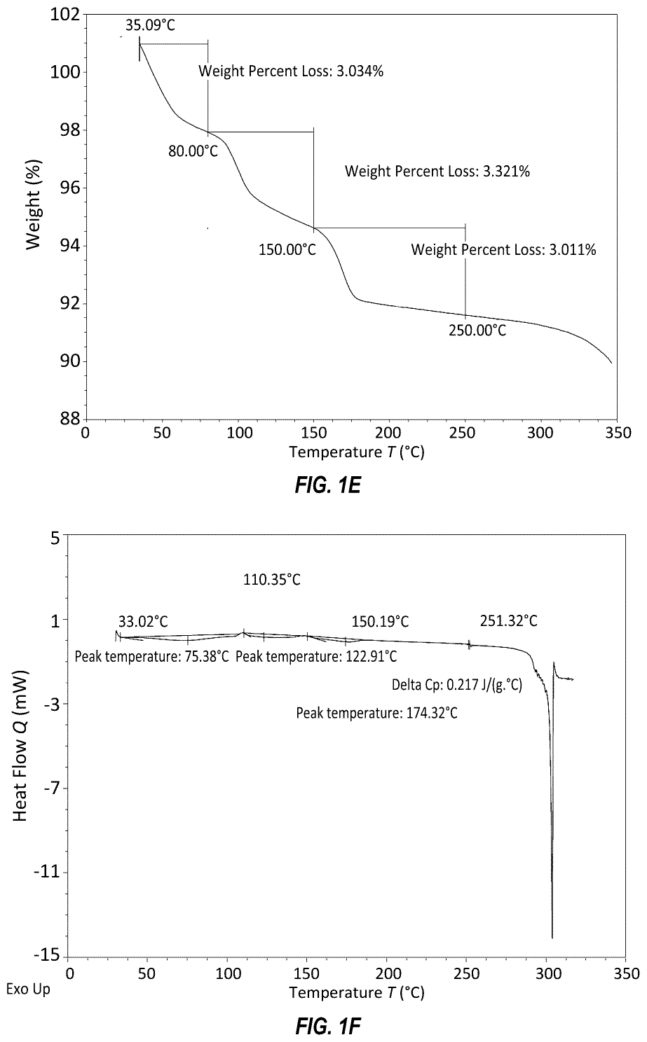

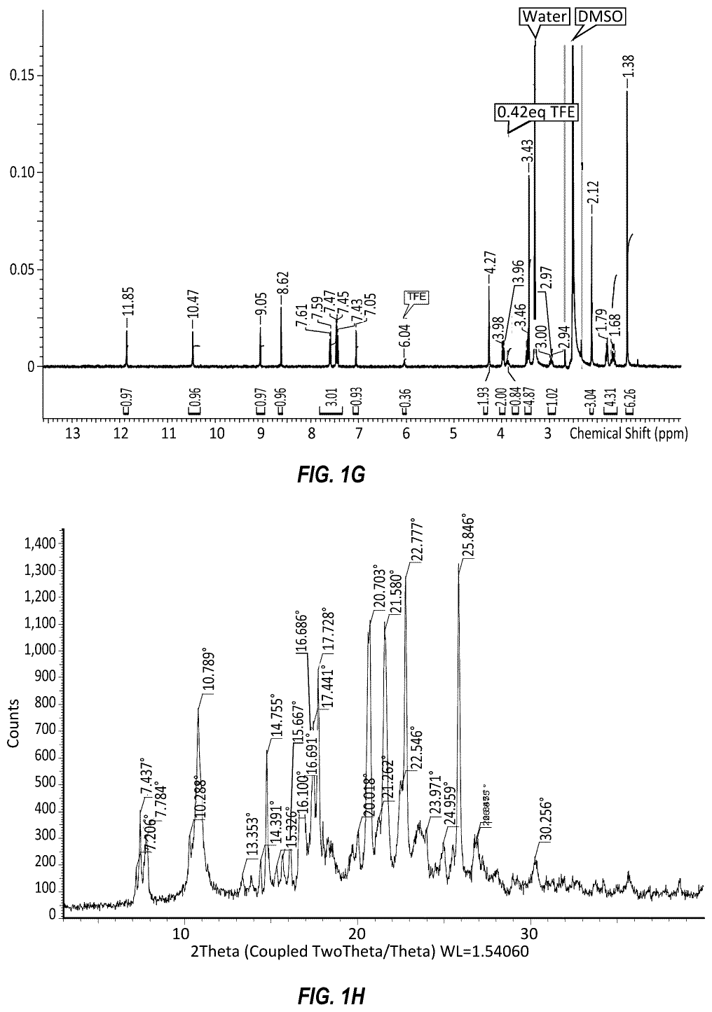

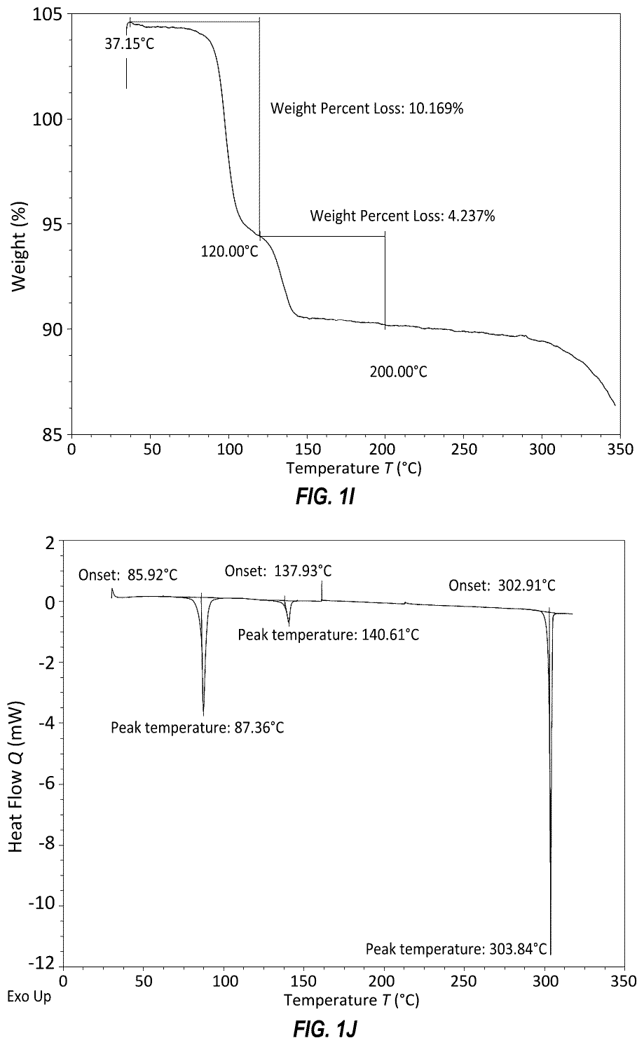

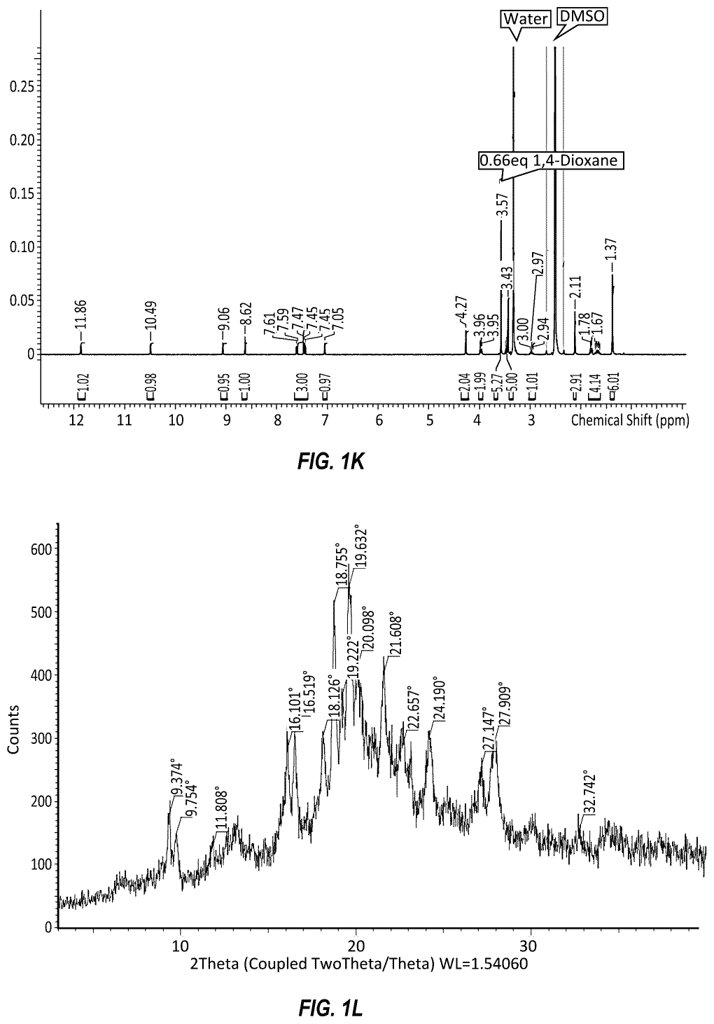

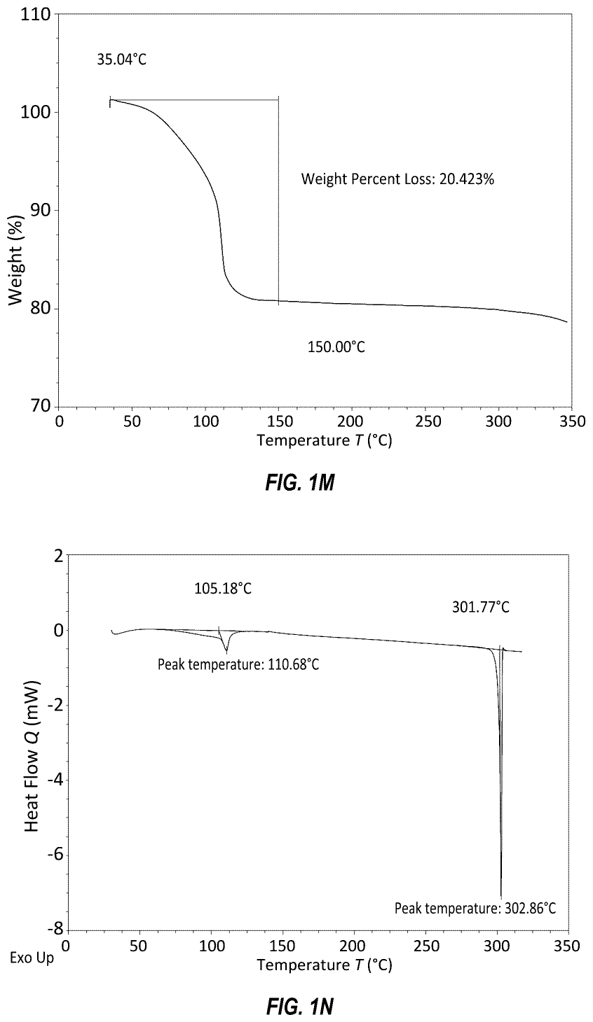

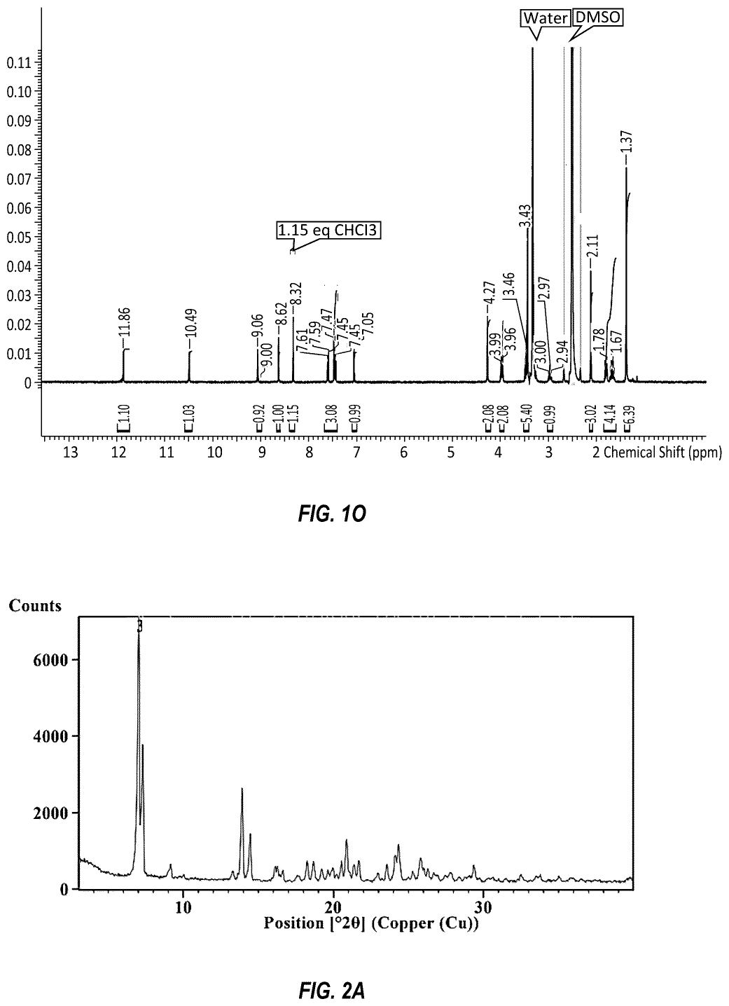

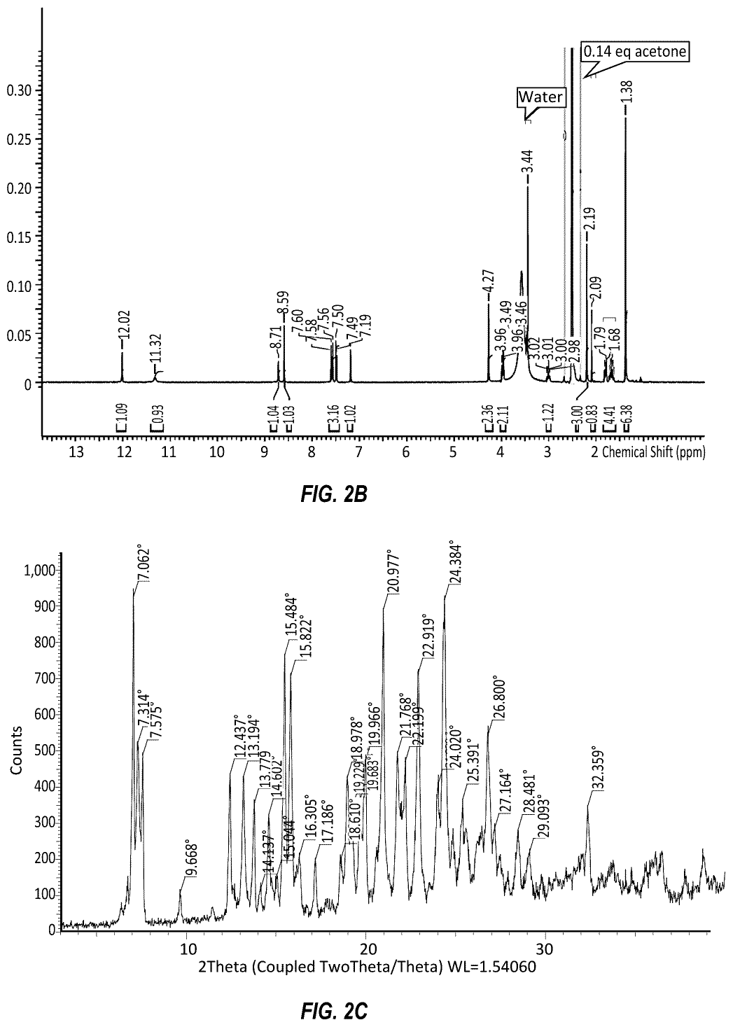

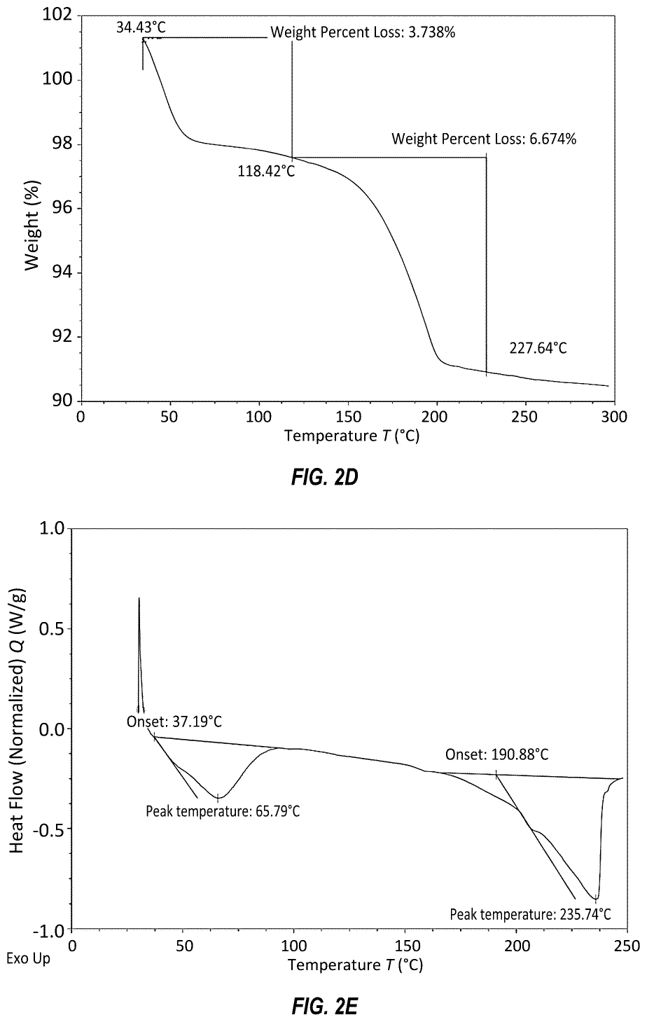

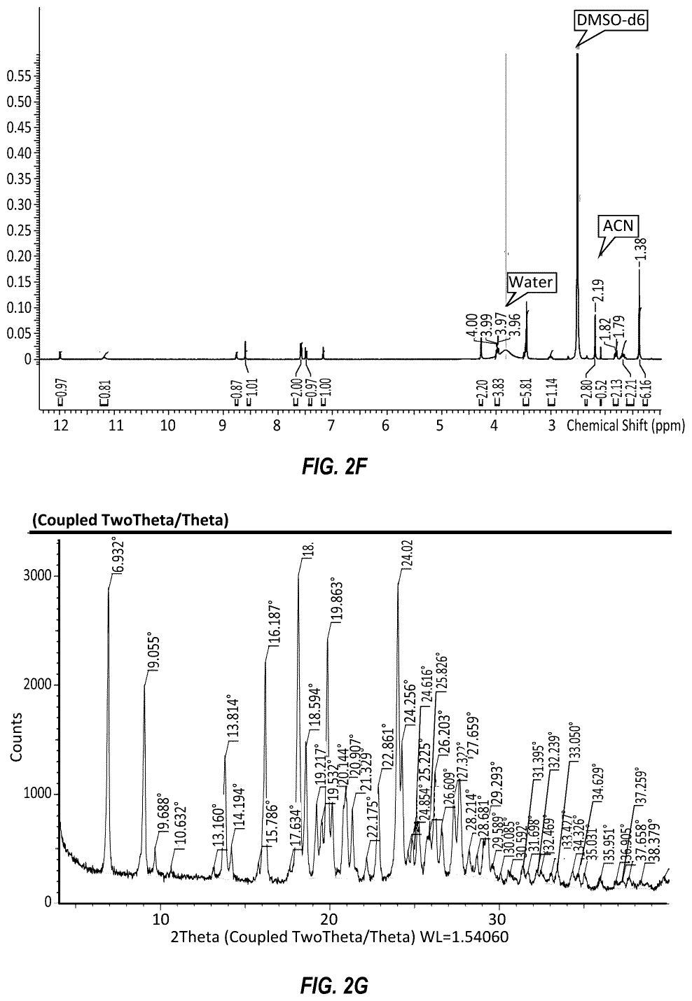

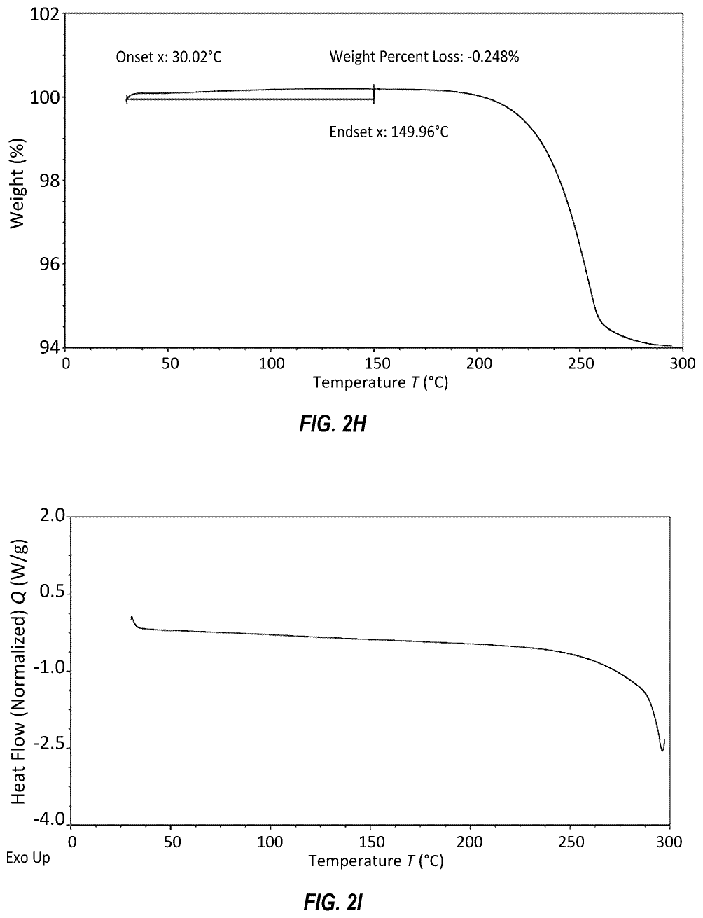

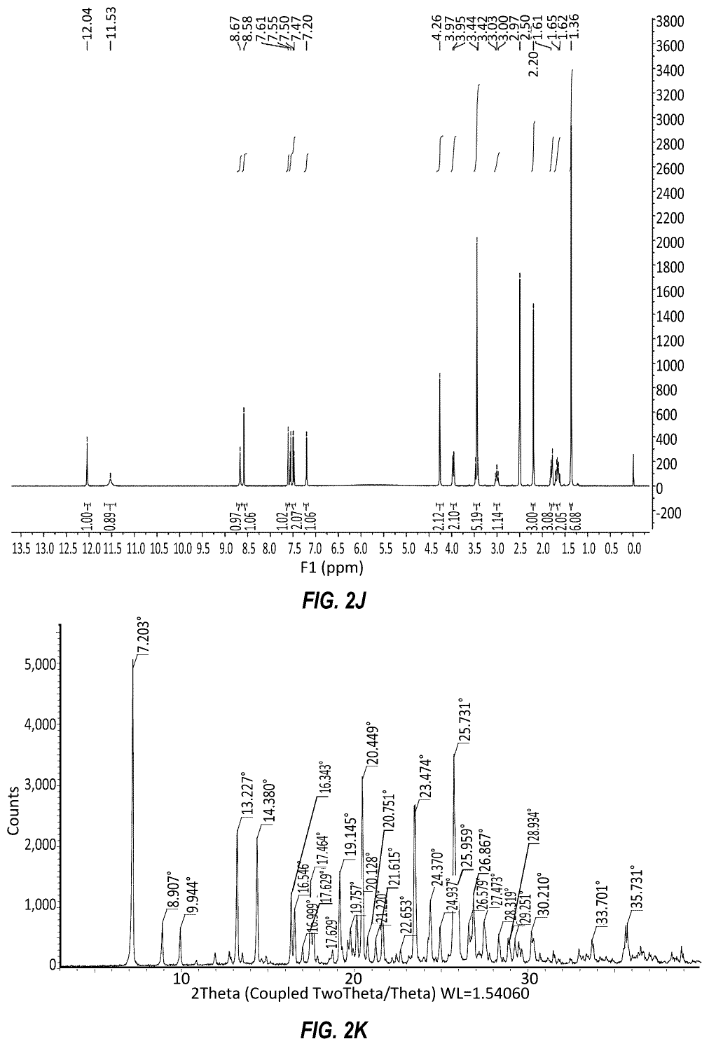

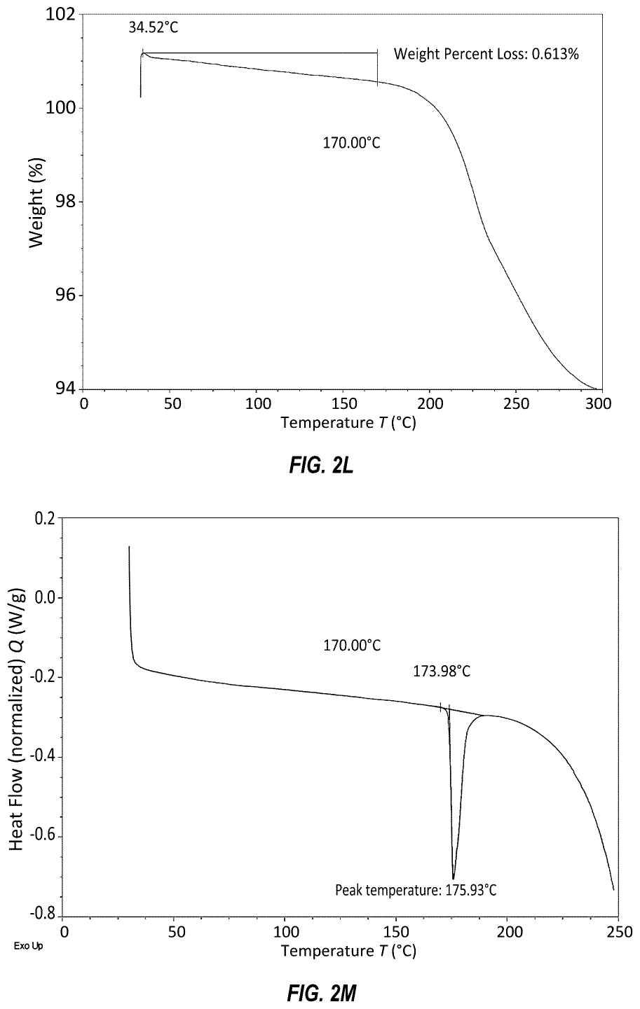

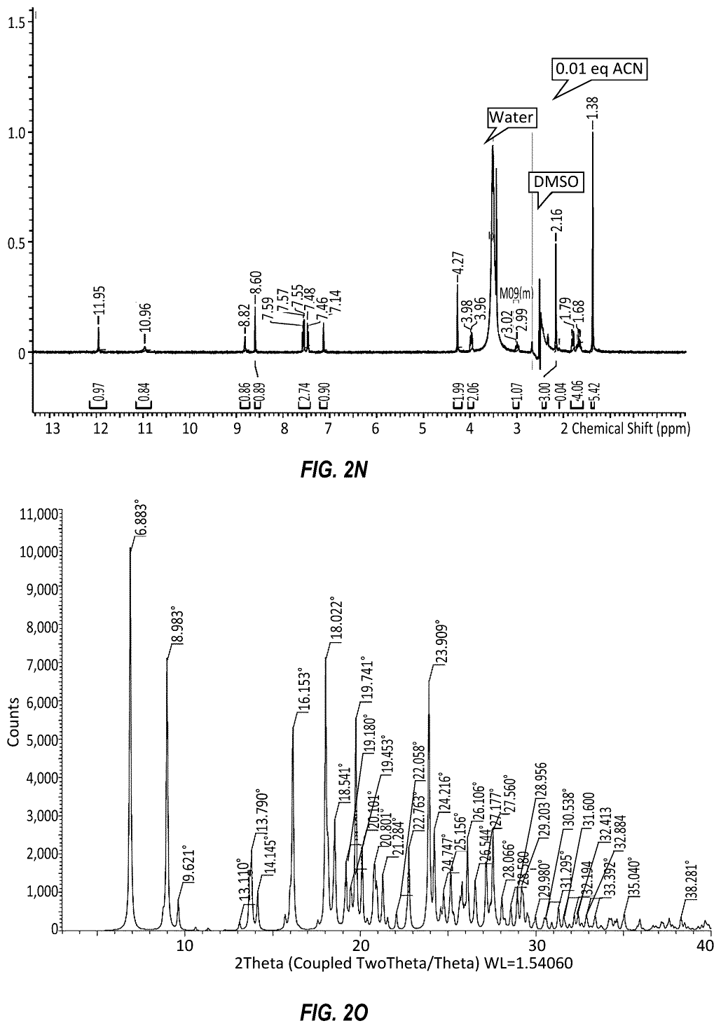

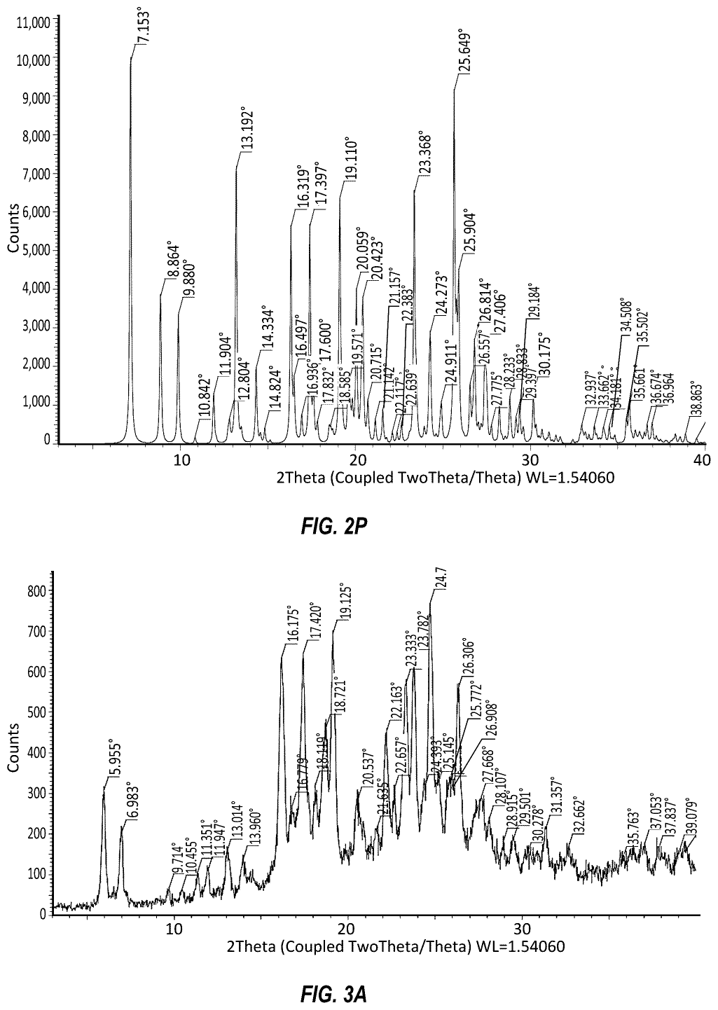

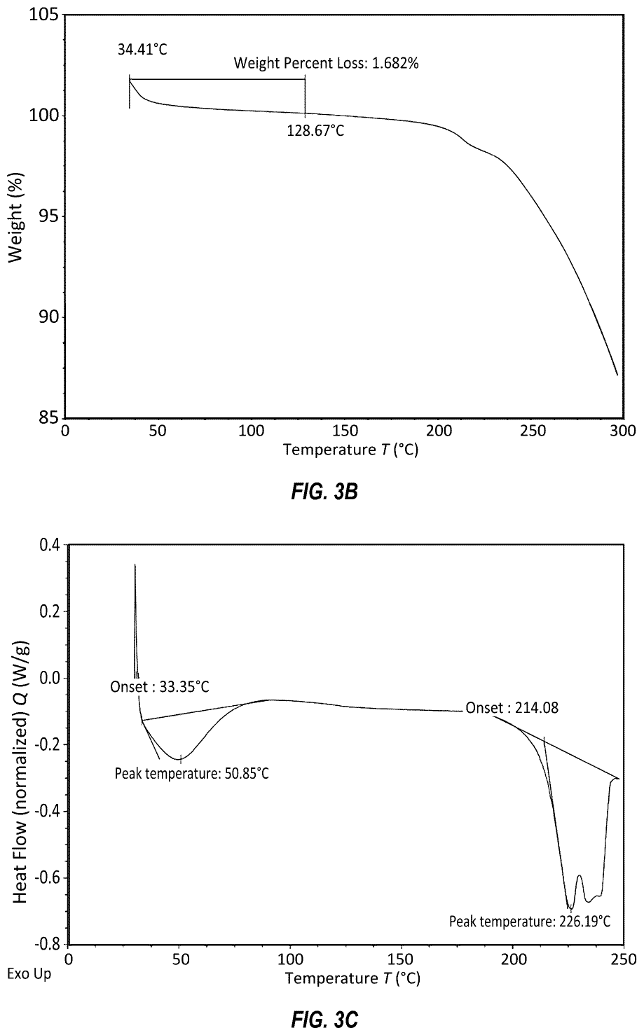

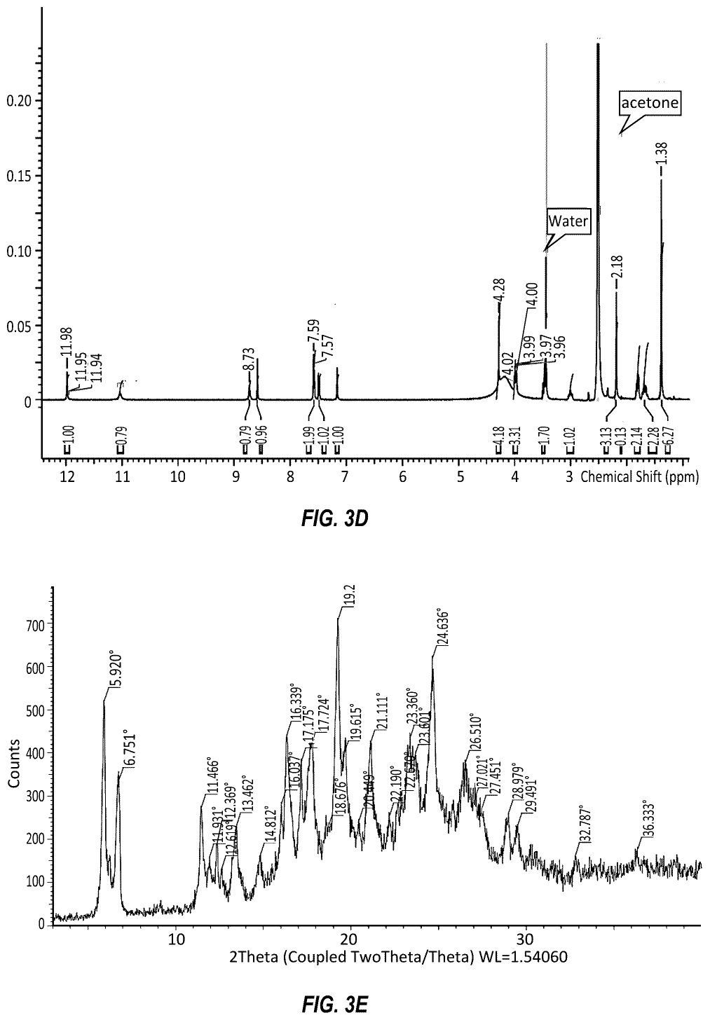

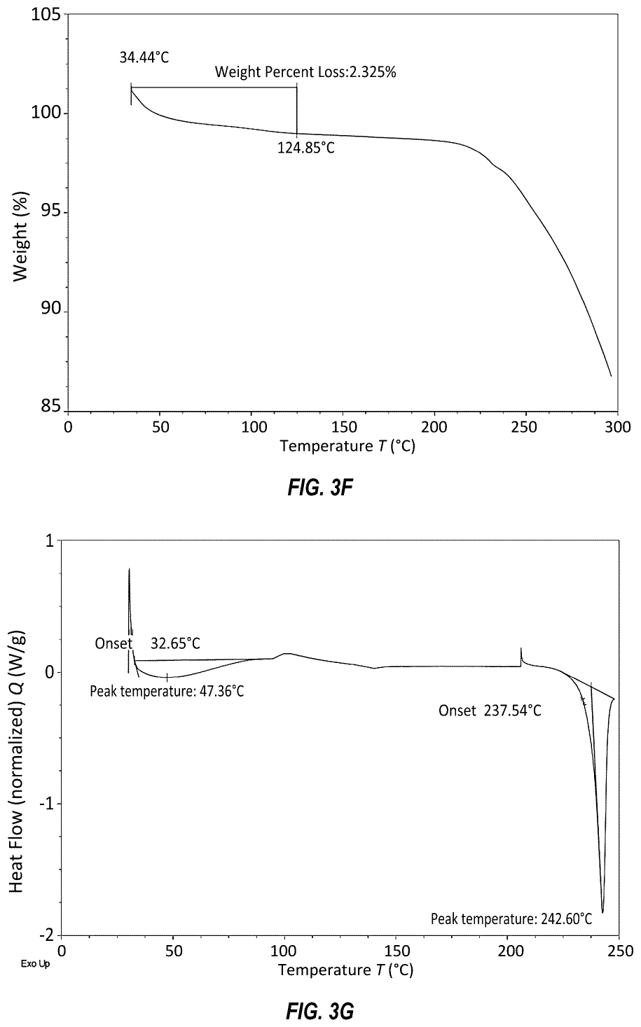

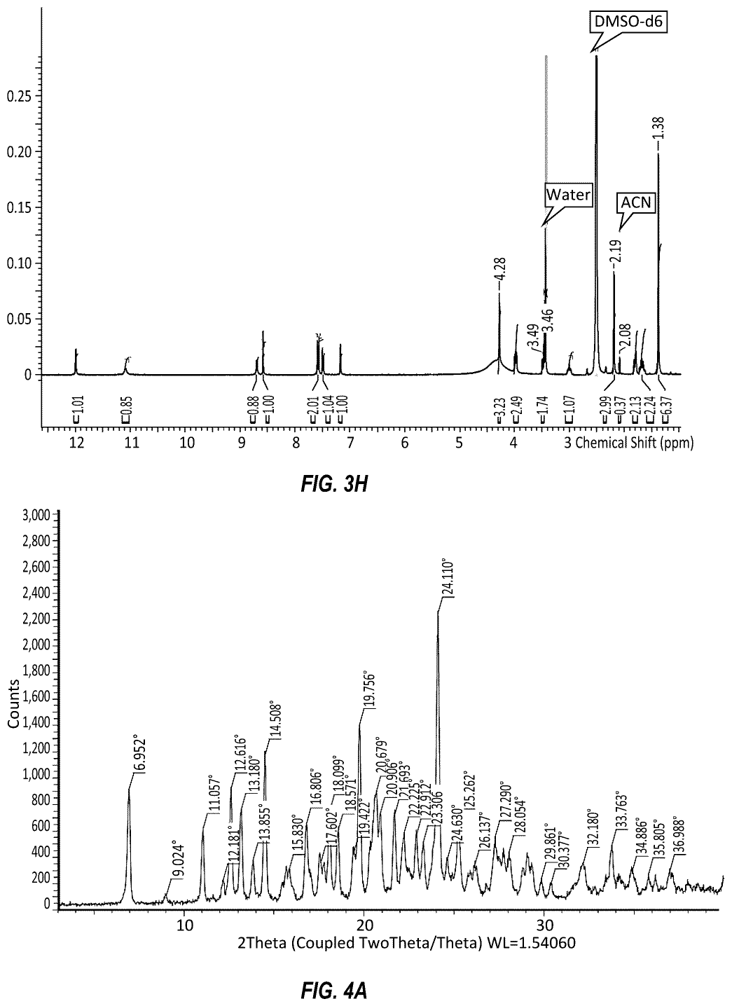

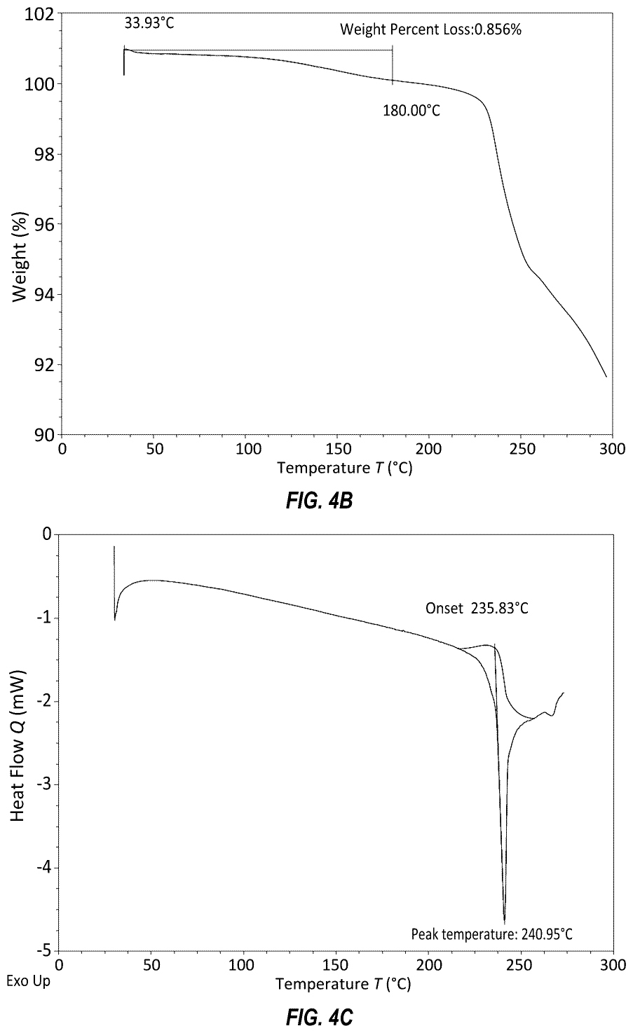

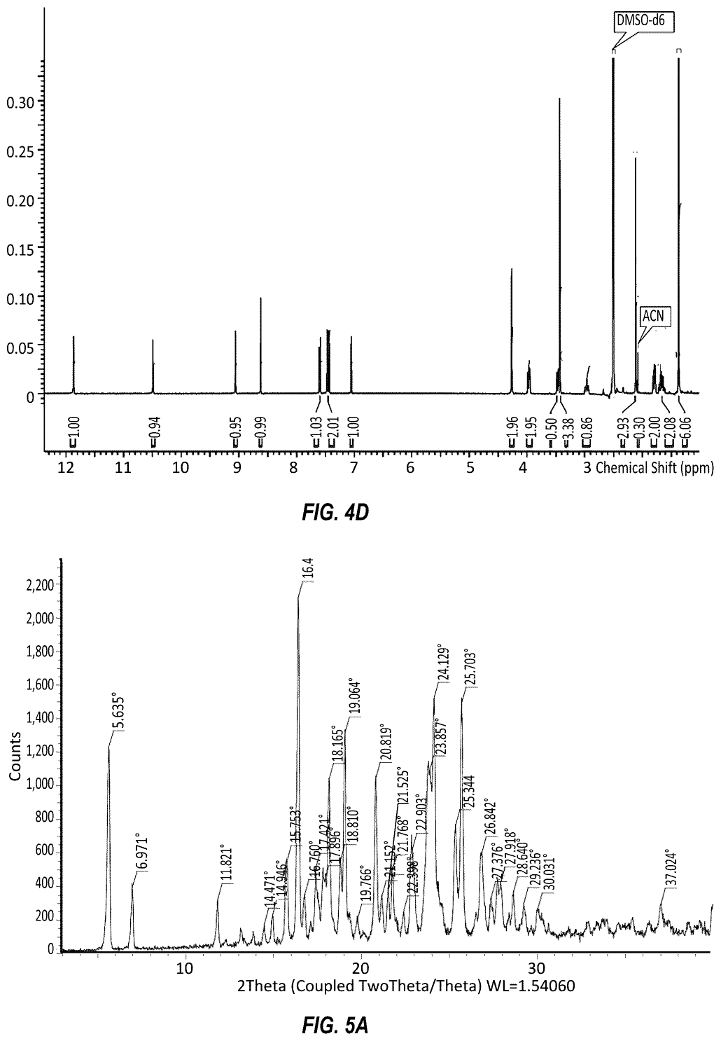

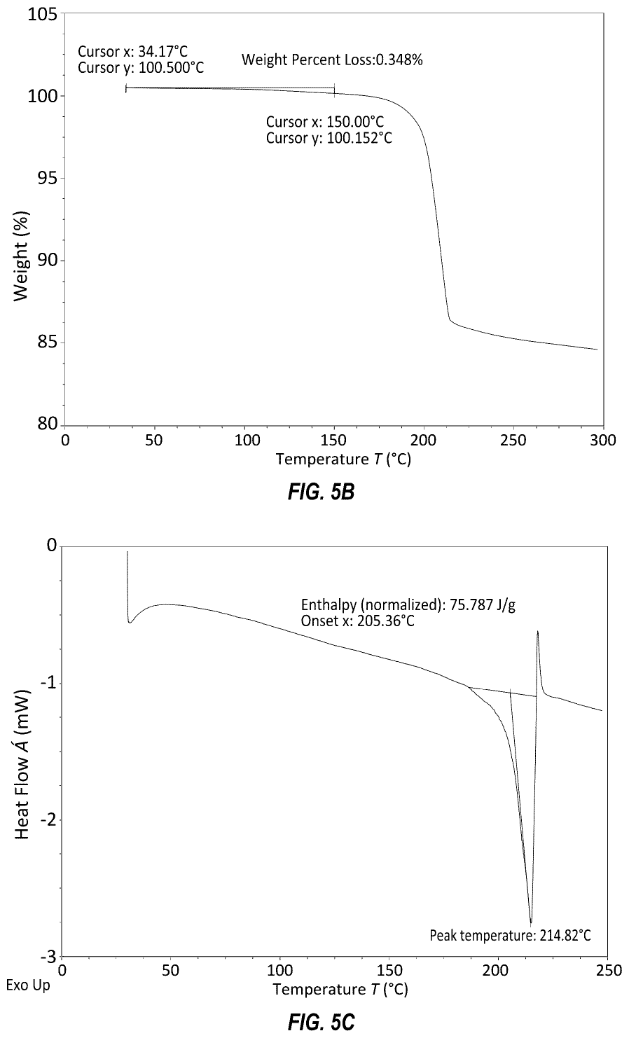

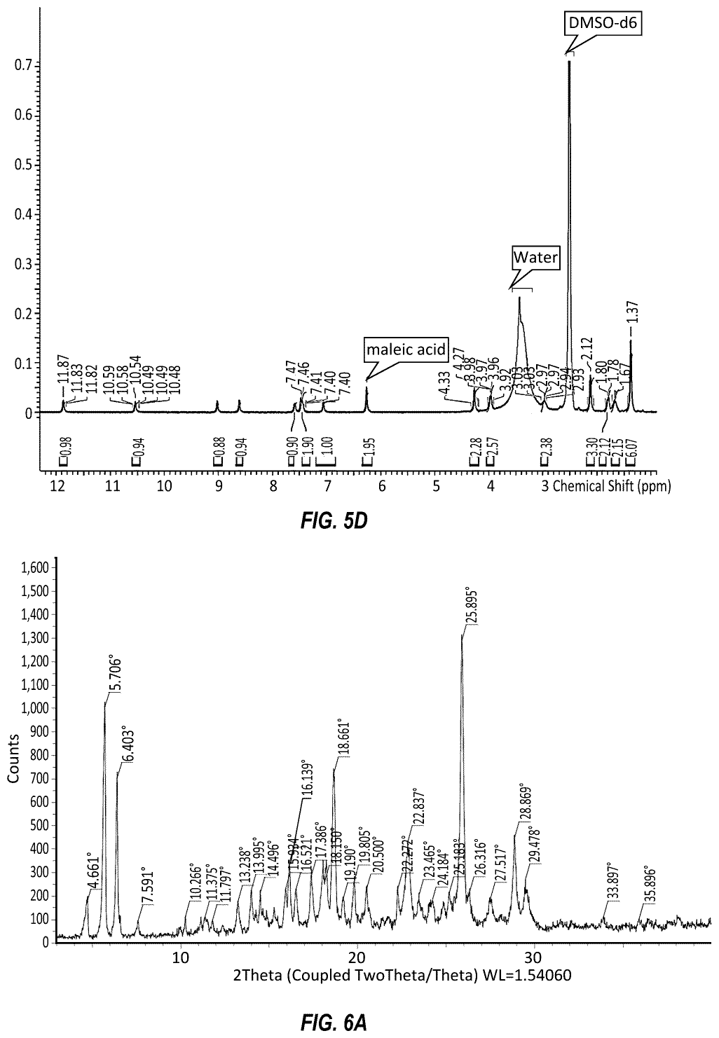

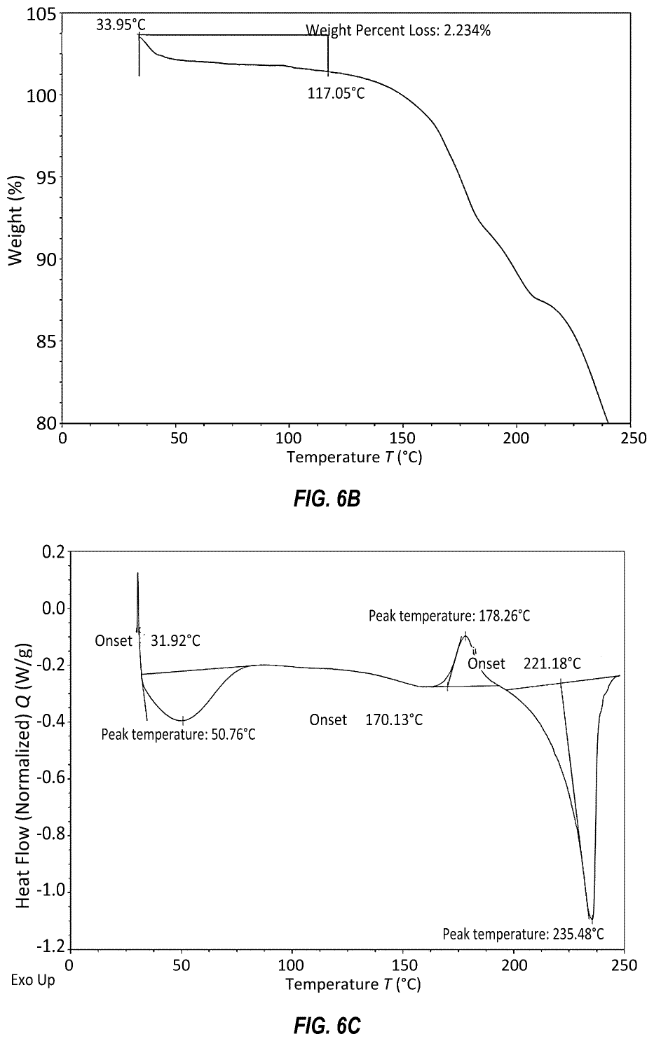

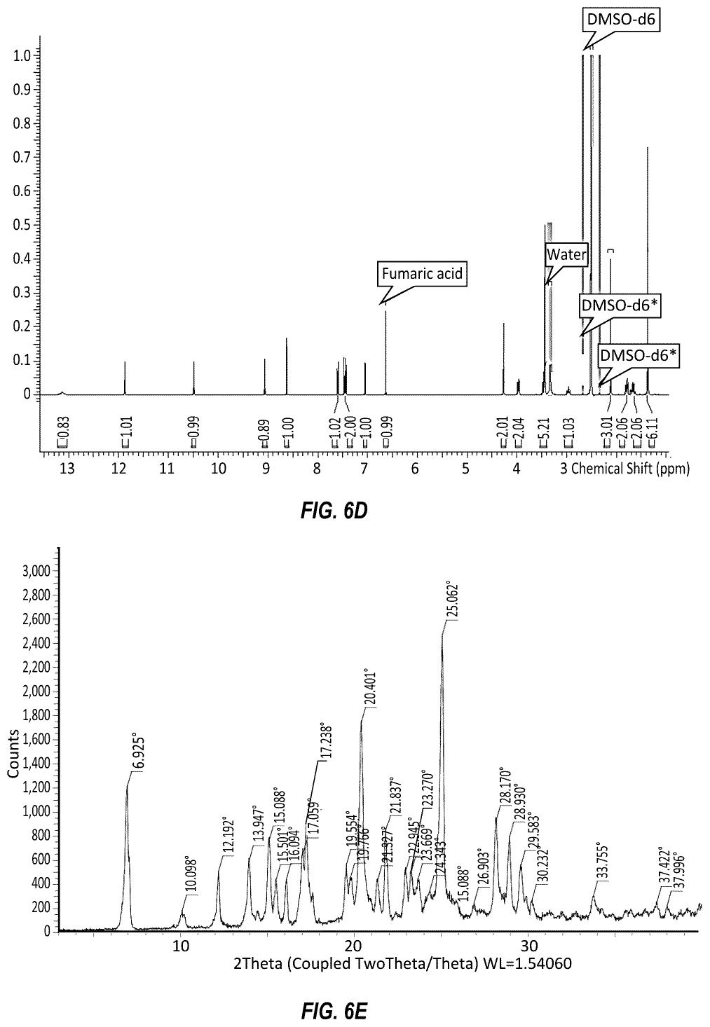

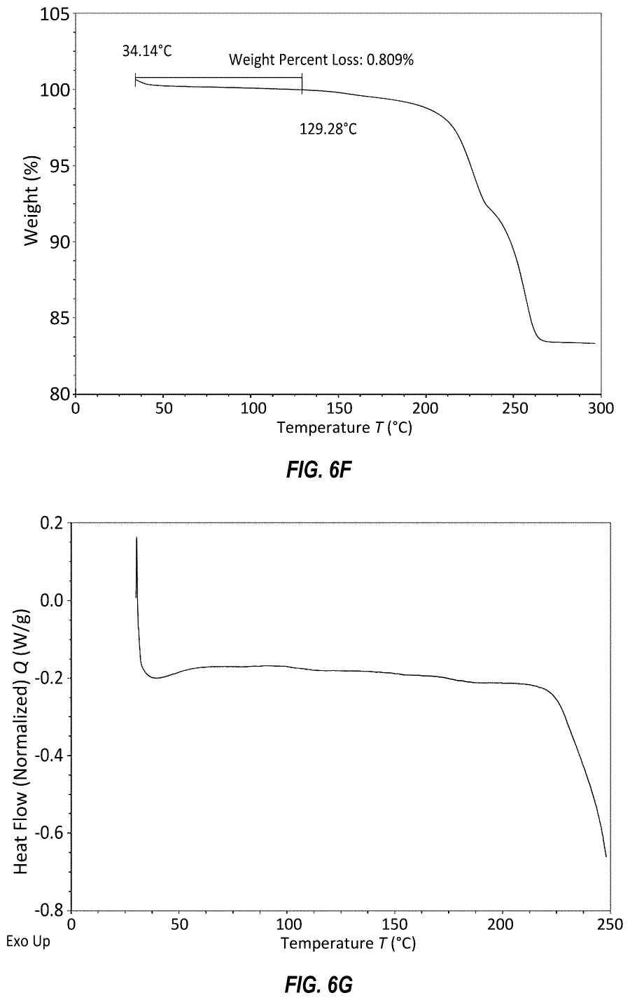

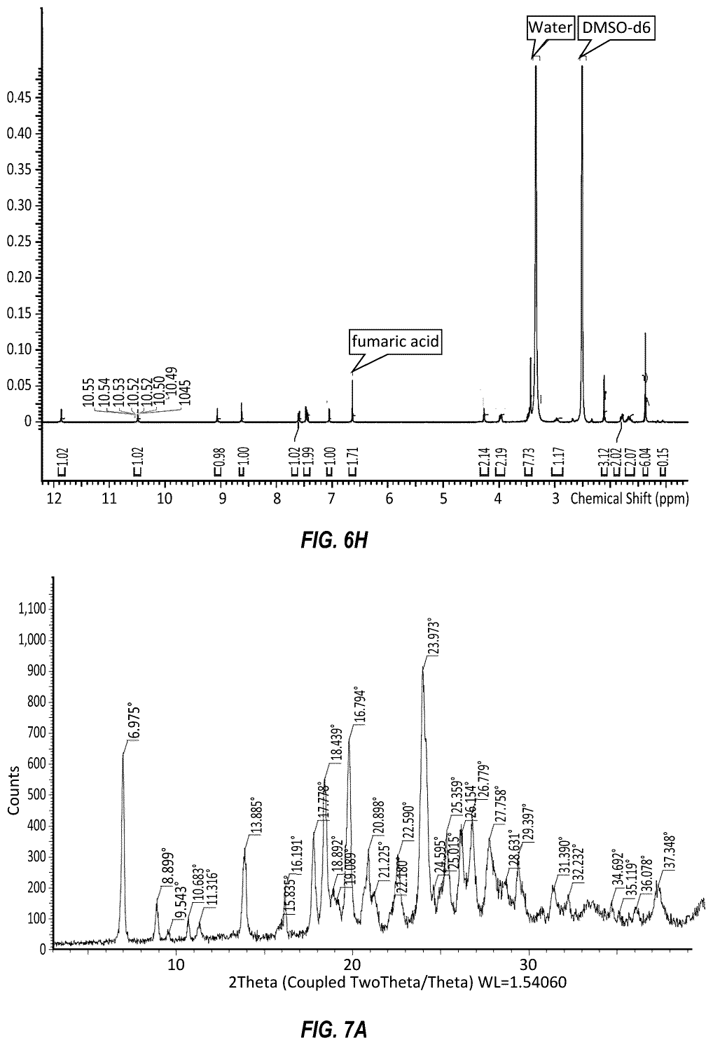

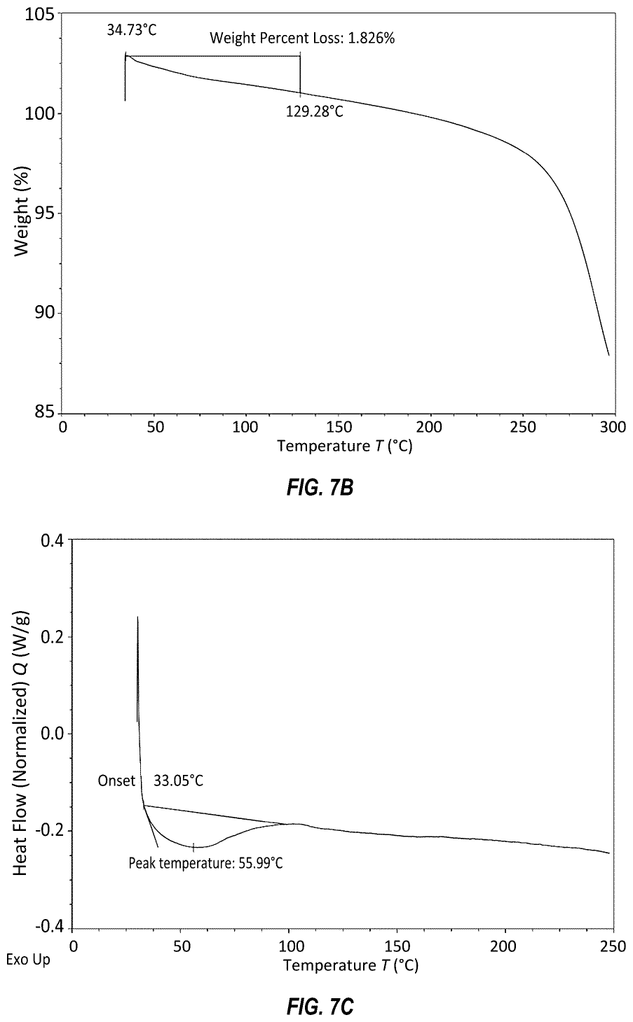

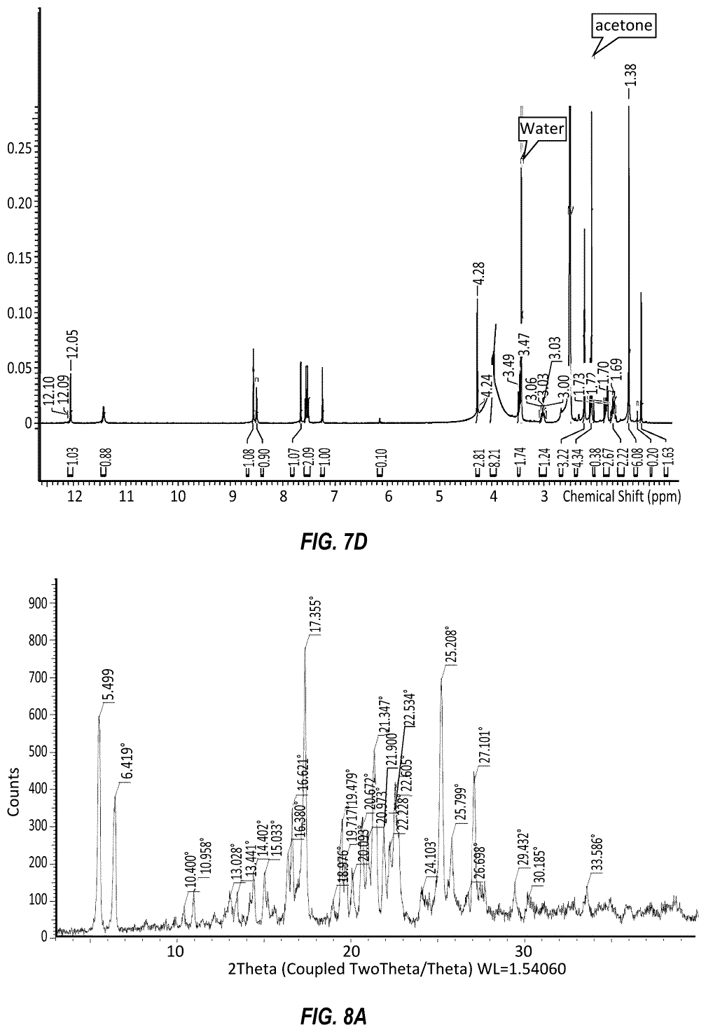

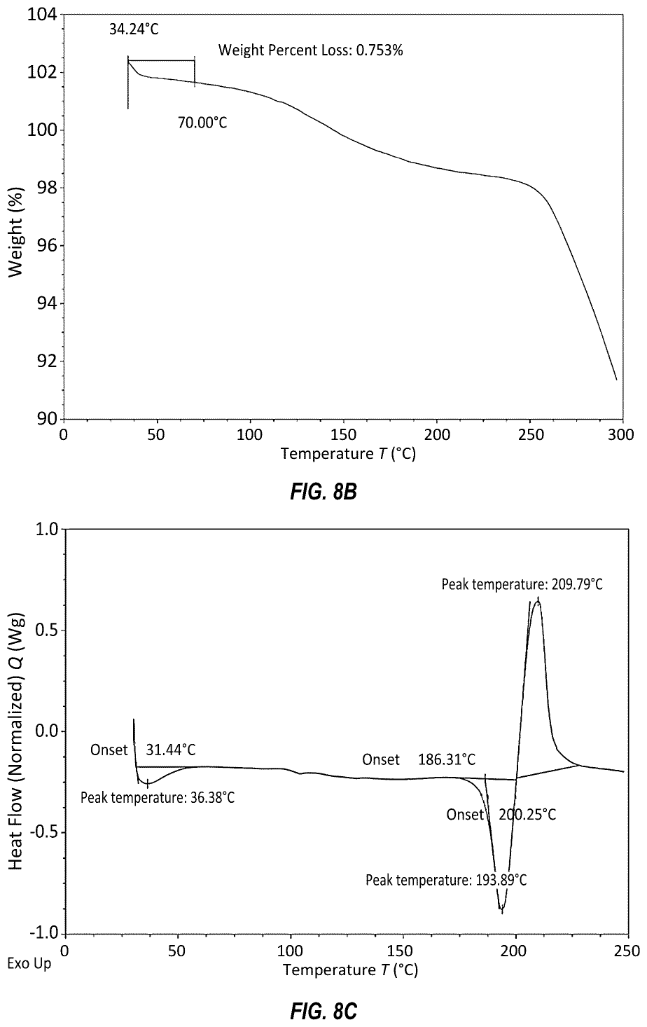

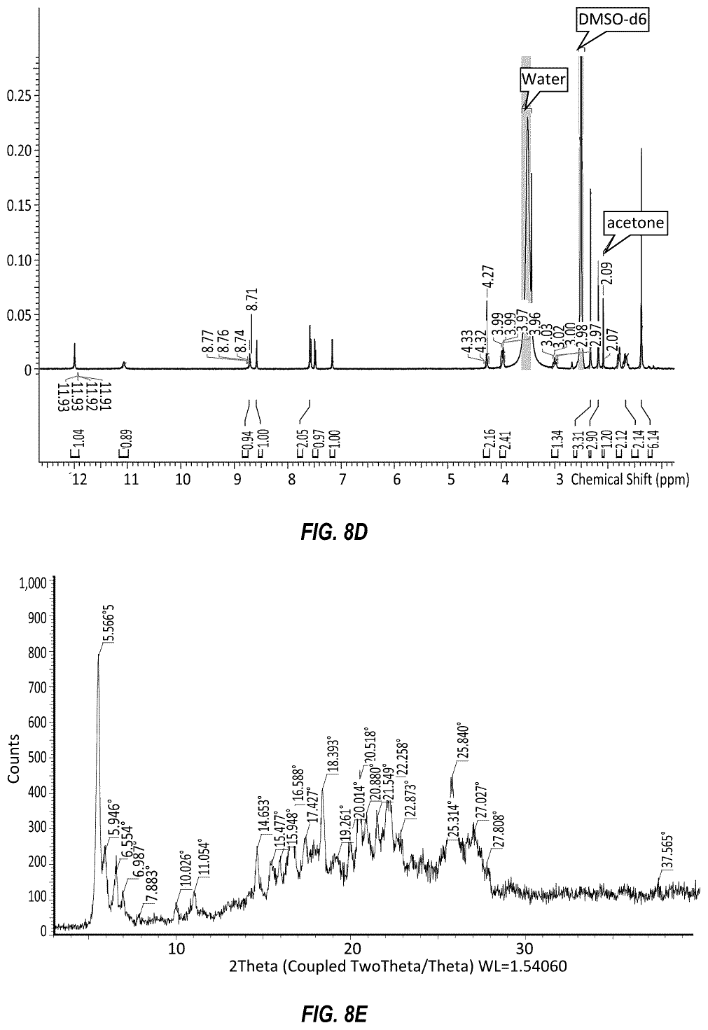

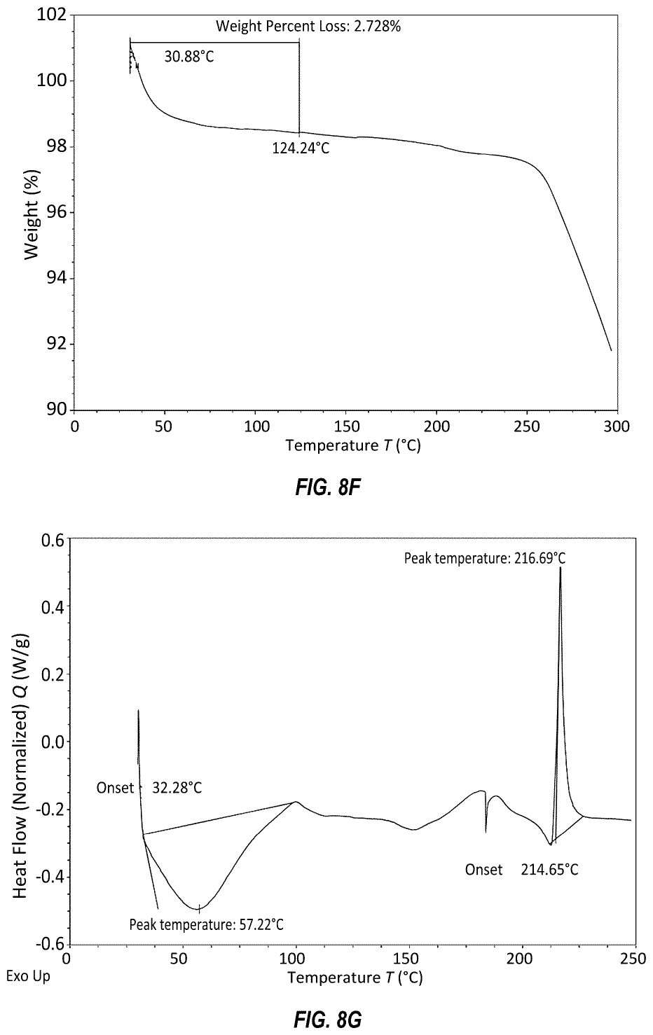

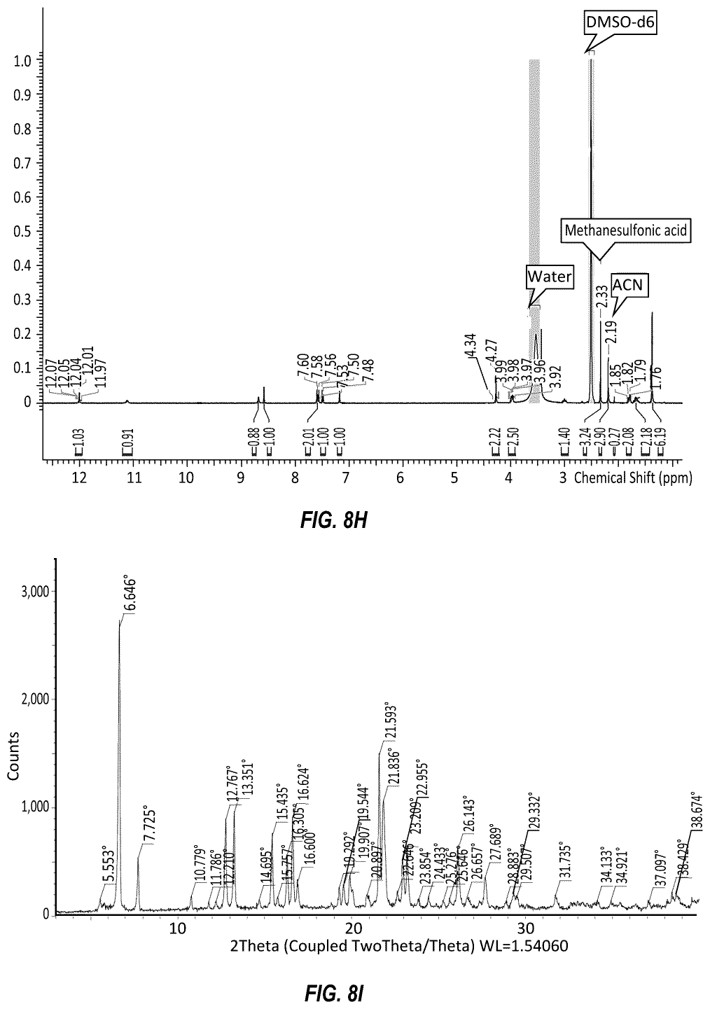

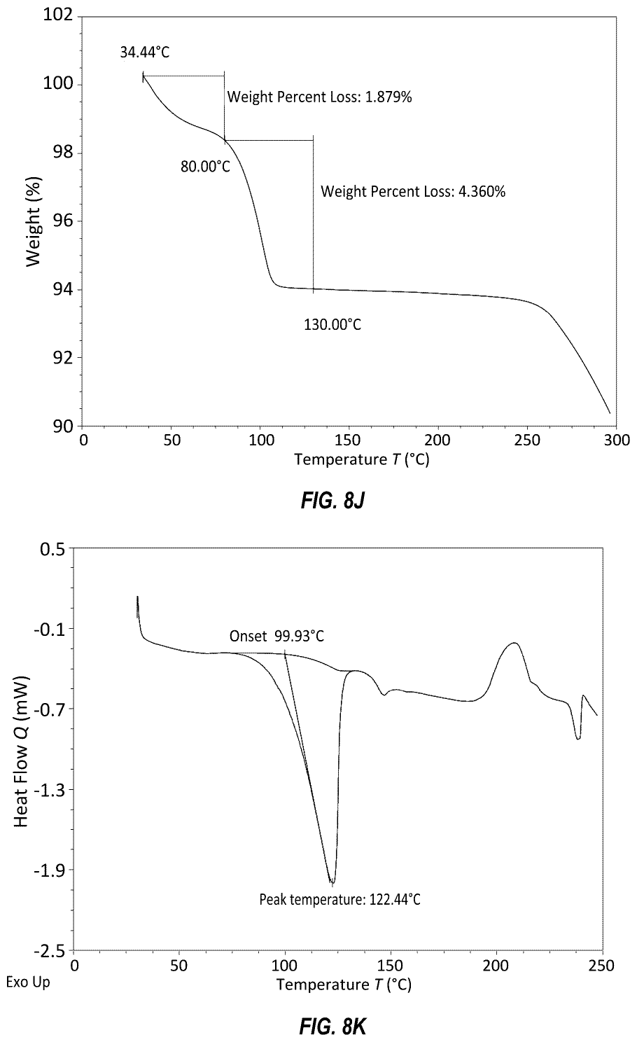

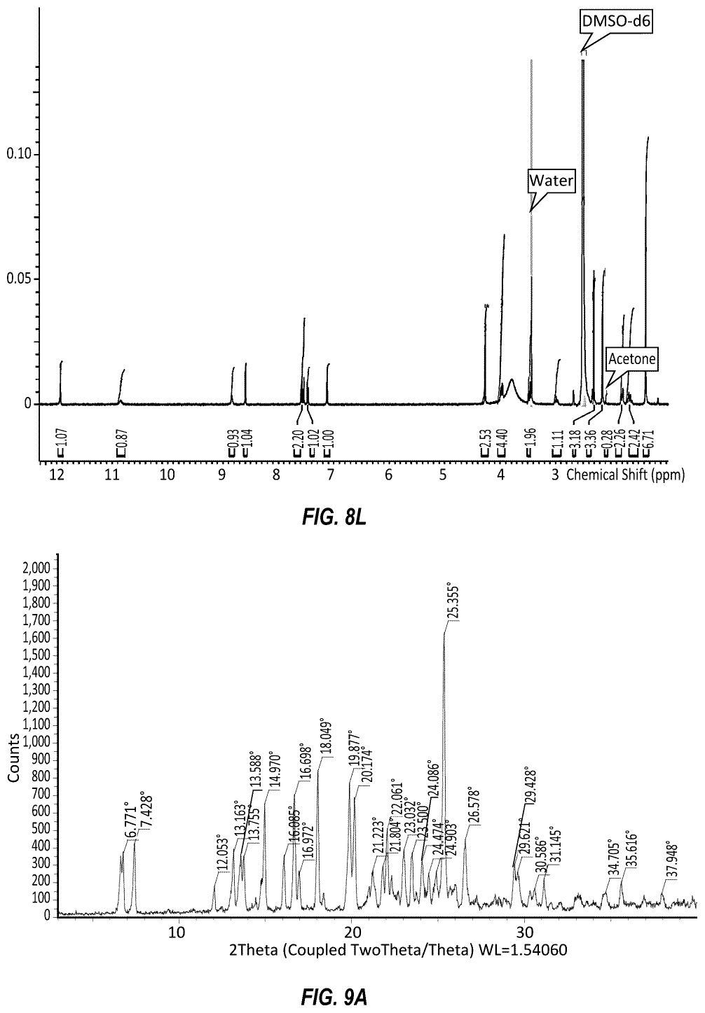

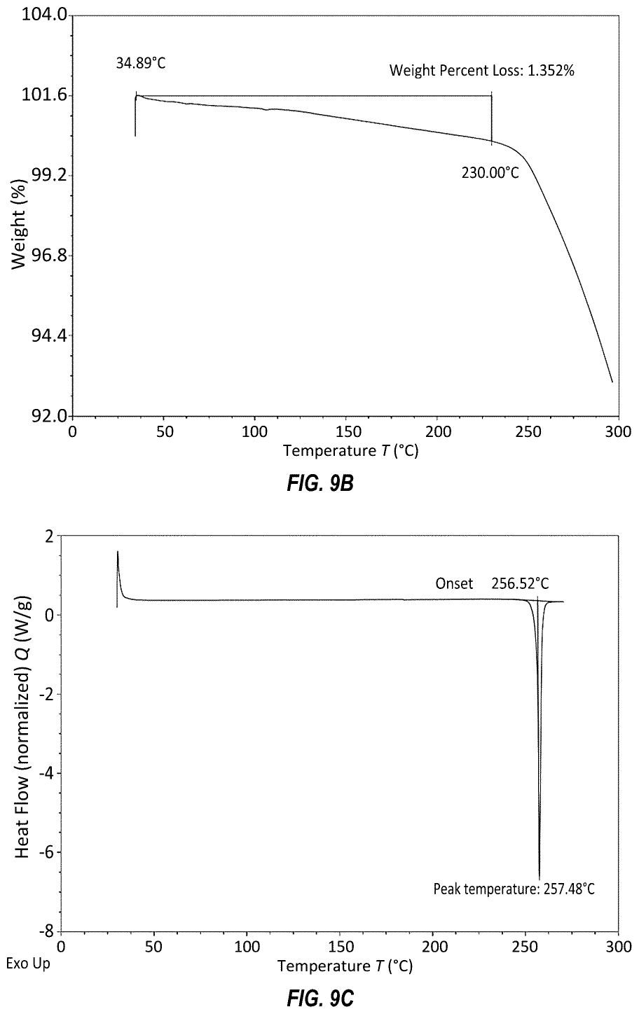

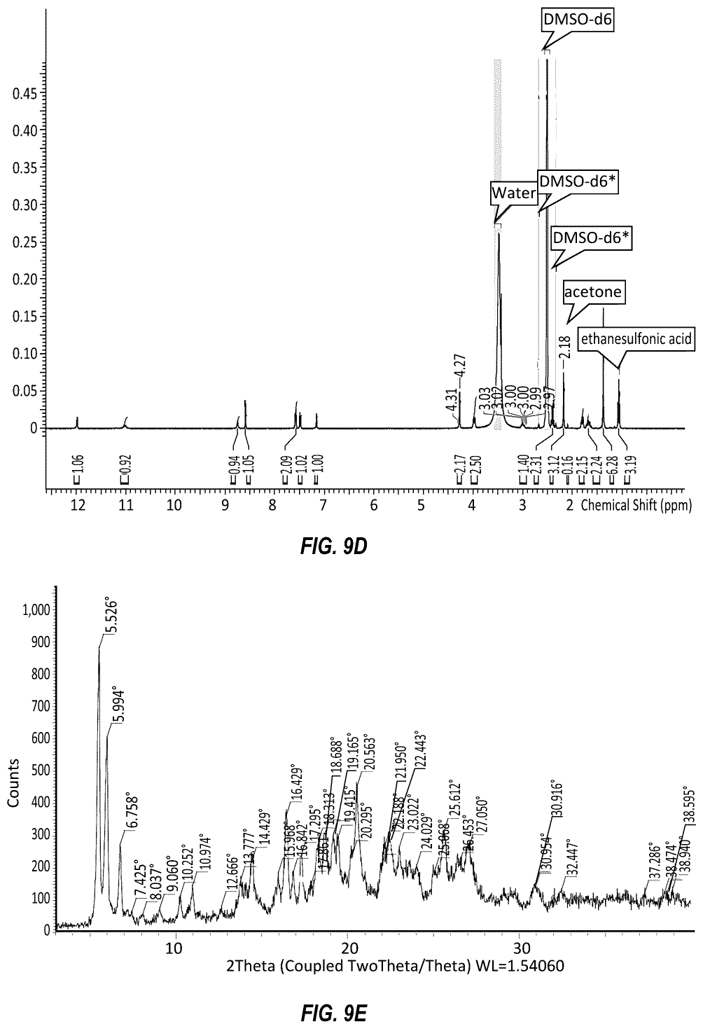

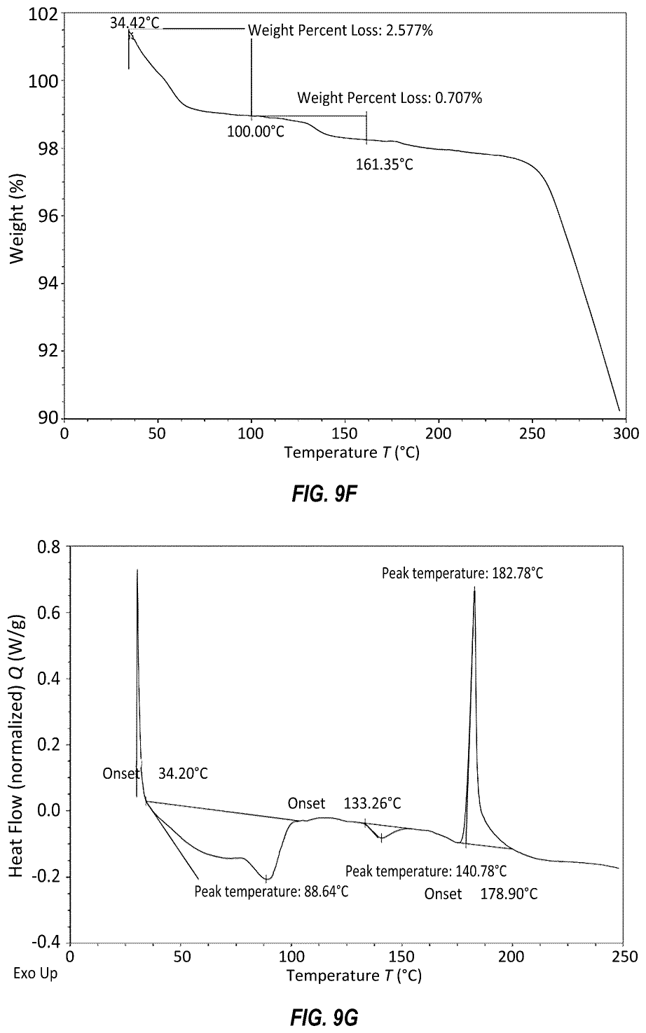

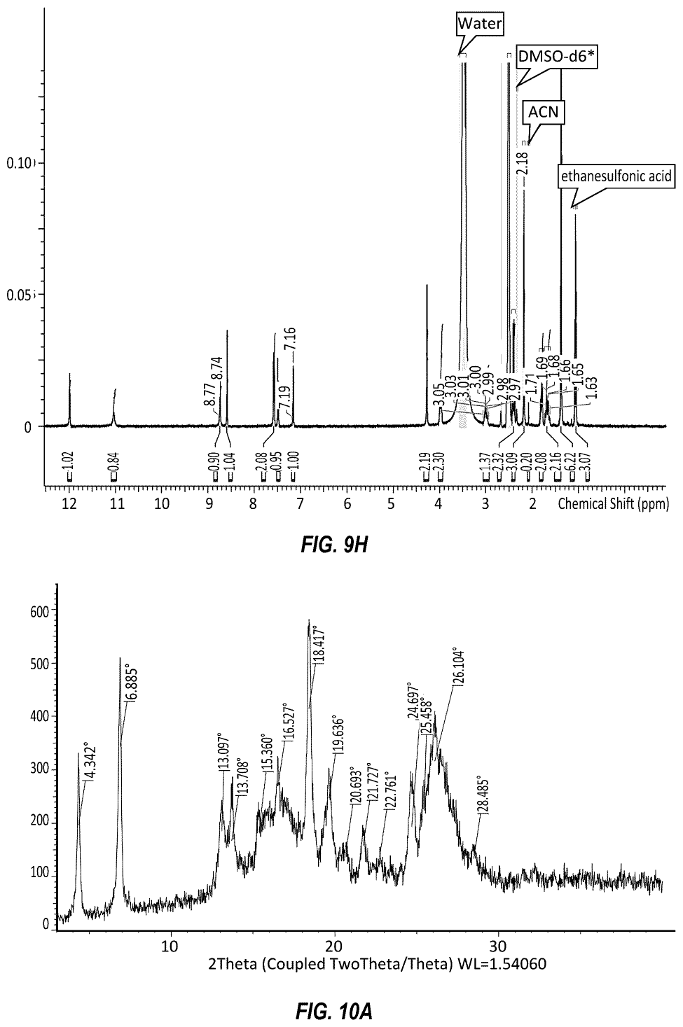

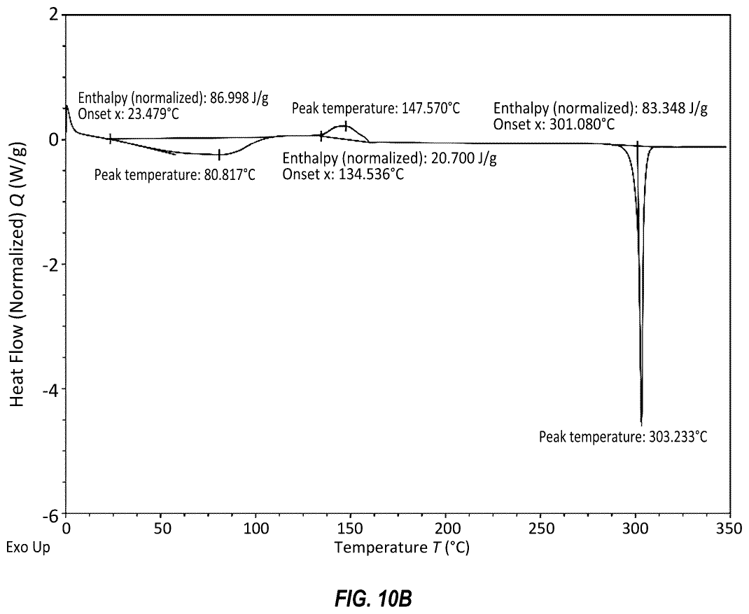

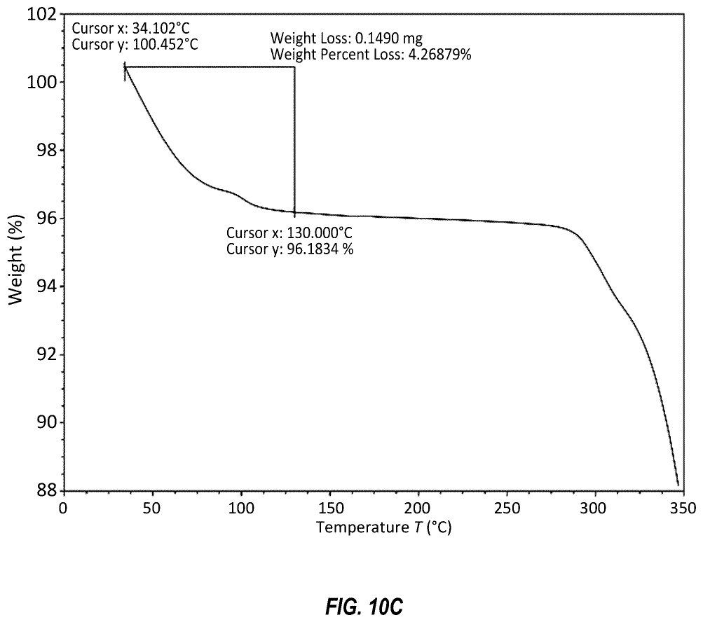

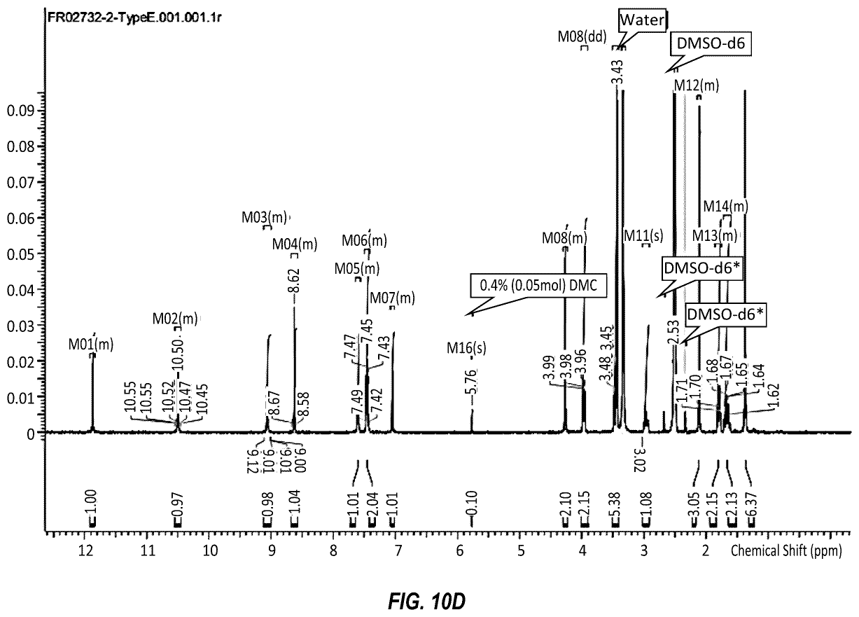

FIG. 1J illustrates a differential scanning calorimetry (DSC) profile of Compound 1 Form C (1,4-dioxane solvate).

FIG. 1K illustrates a ¹H-nuclear magnetic resonance (¹H-NMR) spectrum of Compound 1 Form C (1,4-dioxane solvate).

FIG. 1L illustrates an X-ray powder diffraction (XRPD) pattern of Compound 1 Form D (chloroform solvate).

FIG. 1M illustrates a thermogravimetric analysis (TGA) profile of Compound 1 Form D (chloroform solvate).

FIG. 1N illustrates a differential scanning calorimetry (DSC) profile of Compound 1 Form D (chloroform solvate).

FIG. 1O illustrates a ¹H-nuclear magnetic resonance (¹H-NMR) spectrum of Compound 1 Form D (chloroform solvate).

FIG. 2A illustrates an X-ray powder diffraction (XRPD) pattern of Compound 1 hydrochloride (1:1) Pattern A.

FIG. 2B illustrates a ¹H-nuclear magnetic resonance (¹H-NMR) spectrum of Compound 1 hydrochloride Pattern A.

FIG. 2C illustrates an XRPD overlay pattern of Compound 1 hydrochloride (1:1) Form B.

FIG. 2D illustrates a thermogravimetric analysis (TGA) profile of Compound 1 hydrochloride (1:1) Form B.

FIG. 2E illustrates a differential scanning calorimetry (DSC) profile of Compound 1 hydrochloride (1:1) Form B.

FIG. 2F illustrates a ¹H-nuclear magnetic resonance (¹H-NMR) spectrum of Compound 1 hydrochloride (1:1) Form B.

FIG. 2G illustrates an X-ray powder diffraction (XRPD) pattern of Compound 1 hydrochloride (1:1) Form C.

FIG. 2H illustrates a thermogravimetric analysis (TGA) profile of Compound 1 hydrochloride (1:1) Form C.

FIG. 2I illustrates a differential scanning calorimetry (DSC) profile of Compound 1 hydrochloride (1:1) Form C.

FIG. 2J illustrates a ¹H-nuclear magnetic resonance (¹H-NMR) spectrum of Compound 1hydrochloride (1:1) Form C.

FIG. 2K illustrates an X-ray powder diffraction (XRPD) pattern of Compound 1 hydrochloride (1:1) Form D.

FIG. 2L illustrates a thermogravimetric analysis (TGA) profile of Compound 1 hydrochloride (1:1) Form D.

FIG. 2M illustrates a differential scanning calorimetry (DSC) profile of Compound 1 hydrochloride (1:1) Form D.

FIG. 2N illustrates a ¹H-nuclear magnetic resonance (¹H-NMR) spectrum of Compound 1 hydrochloride (1:1) Form D.

FIG. 2O illustrates the calculated XRPD of the single crystal structure and the simulated XRPD of the single crystal of Compound 1 hydrochloride (1:1) Form C.

FIG. 2P illustrates the calculated XRPD of the single crystal structure and the simulated XRPD of the single crystal of Compound 1 hydrochloride (1:1) Form D.

FIG. 3A illustrates an X-ray powder diffraction (XRPD) pattern of Compound 1 sulfate Form A.

FIG. 3B illustrates a thermogravimetric analysis (TGA) profile of Compound 1 sulfate Form A.

FIG. 3C illustrates a differential scanning calorimetry (DSC) profile of Compound 1 sulfate Form A.

FIG. 3D illustrates a ¹H-nuclear magnetic resonance (¹H-NMR) spectrum of Compound 1 sulfate Form A.

FIG. 3E illustrates an X-ray powder diffraction (XRPD) pattern of Compound 1 sulfate Form B.

FIG. 3F illustrates a thermogravimetric analysis (TGA) profile of Compound 1 sulfate Form B.

FIG. 3G illustrates a differential scanning calorimetry (DSC) profile of Compound 1 sulfate Form B.

FIG. 3H illustrates a ¹H-nuclear magnetic resonance (¹H-NMR) spectrum of Compound 1 sulfate Form B.

FIG. 4A illustrates an X-ray powder diffraction (XRPD) pattern of Compound 1 phosphate Form A.

FIG. 4B illustrates a thermogravimetric analysis (TGA) profile of Compound 1 phosphate Form A.

FIG. 4C illustrates a differential scanning calorimetry (DSC) profile of Compound 1 phosphate Form A.

FIG. 4D illustrates a ¹H-nuclear magnetic resonance (¹H-NMR) spectrum of Compound 1 phosphate Form A.

FIG. 5A illustrates an X-ray powder diffraction (XRPD) pattern of Compound 1 maleate (1:1.0, anhydrate) Form A.

FIG. 5B illustrates a thermogravimetric analysis (TGA) profile of Compound 1 maleate (1:1.0. anhydrate) Form A.

FIG. 5C illustrates a differential scanning calorimetry (DSC) profile of Compound 1 maleate (1:1.0, anhydrate) Form A.

FIG. 5D illustrates a ¹H-nuclear magnetic resonance (¹H-NMR) spectrum of Compound 1 maleate (1:1.0, anhydrate) Form A.

FIG. 6A illustrates an X-ray powder diffraction (XRPD) pattern of Compound 1 fumarate Form A.

FIG. 6B illustrates a thermogravimetric analysis (TGA) profile of Compound 1 fumarate Form A.

FIG. 6C illustrates a differential scanning calorimetry (DSC) profile of Compound 1 fumarate Form A.

FIG. 6D illustrates a ¹H-nuclear magnetic resonance (¹H-NMR) spectrum of Compound 1 fumarate Form A.

FIG. 6E illustrates an X-ray powder diffraction (XRPD) pattern of Compound 1 fumarate (1:0.5) Form B.

FIG. 6F illustrates a thermogravimetric analysis (TGA) profile of Compound 1 fumarate (1:0.5) Form B.

FIG. 6G illustrates a differential scanning calorimetry (DSC) profile of Compound 1 fumarate (1:0.5) Form B.

FIG. 6H illustrates a ¹H-nuclear magnetic resonance (¹H-NMR) spectrum of Compound 1 fumarate (1:0.5) Form B.

FIG. 7A illustrates an X-ray powder diffraction (XRPD) pattern of Compound 1 HBr salt Form A (an anhydrate).

FIG. 7B illustrates a thermogravimetric analysis (TGA) profile of Compound 1 HBr salt Form A (an anhydrate).

FIG. 7C illustrates a differential scanning calorimetry (DSC) profile of Compound 1 HBr salt Form A (an anhydrate).

FIG. 7D illustrates a ¹H-nuclear magnetic resonance (¹H-NMR) spectrum of Compound 1 HBr salt Form A (an anhydrate).

FIG. 8A illustrates an X-ray powder diffraction (XRPD) pattern of Compound 1 mesylate Form A.

FIG. 8B illustrates a thermogravimetric analysis (TGA) profile of Compound 1 mesylate Form A.

FIG. 8C illustrates a differential scanning calorimetry (DSC) profile of Compound 1 mesylate Form A.

FIG. 8D illustrates a ¹H-nuclear magnetic resonance (¹H-NMR) spectrum of Compound 1 mesylate Form A.

FIG. 8E illustrates an X-ray powder diffraction (XRPD) pattern of Compound 1 mesylate Form B.

FIG. 8F illustrates a thermogravimetric analysis (TGA) profile of Compound 1 mesylate Form B.

FIG. 8G illustrates a differential scanning calorimetry (DSC) profile of Compound 1 mesylate Form B.

FIG. 8H illustrates a ¹H-nuclear magnetic resonance (¹H-NMR) spectrum of Compound 1 mesylate Form B.

FIG. 8I illustrates an X-ray powder diffraction (XRPD) pattern of Compound 1 mesylate Form C.

FIG. 8J illustrates a thermogravimetric analysis (TGA) profile of Compound 1 mesylate Form C.

FIG. 8K illustrates a differential scanning calorimetry (DSC) profile of Compound 1 mesylate Form C.

FIG. 8L illustrates a ¹H-nuclear magnetic resonance (¹H-NMR) spectrum of Compound 1 mesylate Form C.

FIG. 9A illustrates an X-ray powder diffraction (XRPD) pattern of Compound 1 esilate Form A.

FIG. 9B illustrates a thermogravimetric analysis (TGA) profile of Compound 1 esilate Form A.

FIG. 9C illustrates a differential scanning calorimetry (DSC) profile of Compound 1 esilate Form A.

FIG. 9D illustrates a ¹H-nuclear magnetic resonance (¹H-NMR) spectrum of Compound 1 esilate Form A.

FIG. 9E illustrates an X-ray powder diffraction (XRPD) pattern of Compound 1 esilate Form B.

FIG. 9F illustrates a thermogravimetric analysis (TGA) profile of Compound 1 esilate Form B.

FIG. 9G illustrates a differential scanning calorimetry (DSC) profile of Compound 1 esilate Form B.

FIG. 9H illustrates a ¹H-nuclear magnetic resonance (¹H-NMR) spectrum of Compound 1 esilate Form B.

FIG. 10A illustrates an X-ray powder diffraction (XRPD) pattern of Compound 1 Form E.

FIG. 10B illustrates a differential scanning calorimetry (DSC) profile of Compound 1 Form E.

FIG. 10C illustrates a thermogravimetric analysis (TGA) profile of Compound 1 Form E.

FIG. 10D illustrates a ¹H-nuclear magnetic resonance (¹H-NMR) spectrum of Compound 1 Form E.

### DETAILED DESCRIPTION OF THE DISCLOSURE

Unless defined otherwise, all technical and scientific terms used herein have the same meaning as is commonly understood by one of skill in the art to which this invention belongs. All patents, patent applications, and publications referred to herein are incorporated by reference.

As used herein, the term “solvate” refers to a crystalline form of Compound 1 which contains solvent.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject is suspected of having a multi-tyrosine kinase-associated cancer.

As used herein, a “therapeutically effective amount” of a crystalline Form of a salt of Compound 1 is an amount that is sufficient to ameliorate, or in some manner reduce a symptom or stop or reverse the progression of a condition, or negatively modulate or inhibit the activity of a multi-tyrosine kinase. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used in reference to XRPD peak positions refers to the inherent variability of peaks depending on the calibration of the instrument, processes used to prepare the crystalline Forms of the present invention, age of the crystalline Forms, and the type of instrument used in the analysis. The variability of the instrumentation used for XRPD analysis was about ±0.2°2θ.

As used herein, the term “about” when used in reference to DSC endothermic peak onset refers to the inherent variability of peaks depending on the calibration of the instrument, method used to prepare the samples of the present invention and the type of instrument used in the analysis. The variability of the instrumentation used for DSC analysis was ±5° C. prefer ±2° C.

As used herein, the term “about” when used in reference to reaction conditions or procedure, the variability of the temperature was about ±5° C.

### GENERAL METHODS

The general methods outlined below were used in the exemplified Examples unless otherwise noted.

### Crystallization Techniques

Crystalline Forms disclosed herein may be prepared using a variety of methods well known to those skilled in the art including crystallization or recrystallization from a suitable solvent or by sublimation. A wide variety of techniques may be employed, including those in the exemplified Examples, for crystallization or recrystallization including evaporation of a water-miscible or a water-immiscible solvent or solvent mixture, crystal seeding in a supersaturated solution, decreasing the temperature of the solvent mixture, or freeze-drying the solvent mixture.

Crystallization disclosed herein may be done with or without crystal seed. The crystal seed may come from any previous batch of the desired crystalline Form.

### Instruments and Parameters

For XRPD analysis without special instructions, Bruker D8 Advance with LYNXEYE_XE_T (1D mode) as a detector was used to characterize all the physical Forms obtained in the present disclosure, without special instructions. The XRPD parameters used are listed as follows:

| | |
|---|---|
| Radiation | Cu, kα, Kα1 (Å): 1.5406Å or 1.5418Å |
| X-ray generator power | 40 kV, 40 mA |

TGA and DSC were used to characterize the physical Forms obtained in the present disclosure, without special instructions, wherein TGA data were collected using Discovery 5500 or Q5000 Instruments; and, DSC was performed using a TA Discovery 2500 Instrument.

Karl Fischer (KF) was used to test the water content, wherein the data were collected using a Mettler Toledo Coulometric KF Titrator C30 (method was Coulometric) Instrument.

The following Examples are intended to illustrate further certain embodiments of the invention and are not intended to limit the scope of the invention.

### EXAMPLE

### Example 1A: Preparation of N-(5-(2,2-Dimethyl-2,3-Dihydro-[1,4]Dioxino[2,3-B]Pyridin-6-Yl)-4-((6-(Methylsulfonyl)-4-(Tetrahydro-2h-Pyran-4- Yl)Pyridin-2-Yl)Amino)Pyridin-2-Yl)Acetamide (Compound 1)

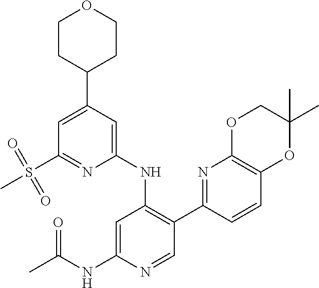

### Method A:

Step 1: 1-(2,6-dibromopyridin-3-yloxy)-2-methylpropan-2-ol

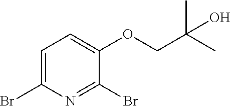

Into a 100-mL round-bottom flask, were placed 2,6-dibromopyridin-3-ol (2.00 g, 7.90 mmol), DMF (30 mL), K₂CO₃ (3.28 g, 23.73 mmol), 2,2-dimethyloxirane (0.68 g, 9.43 mmol). The resulting solution was stirred for 5 hr at 100° C. in an oil bath. After cooled to room temperature, the resulting solution was diluted with 50 mL of H₂O, extracted with 3×30 mL of ethyl acetate and the organic layers were combined and concentrated. The residue was purified by combi-flash (EtOAc/PE=1:4) to give the product (2.2 g, 85% yield). LCMS (ESI, m/z) [M+1]⁺ 324.
Step 2: 6-bromo-2,2-dimethyl-2,3-dihydro-[1,4]dioxino[2,3-b]pyridine

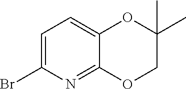

Into a 100-mL round-bottom flask, were placed 1-[(2,6-dibromopyridin-3-yl)oxy]-2-methylpropan-2-ol (2.00 g, 6.15 mmol), DMF (30 mL). This was followed by the addition of NaH (0.49 g, 60% in mineral oil, 12.30 mmol) in portions at 0° C. The resulting solution was stirred for 3 hr at 90° C. in an oil bath. After cooled to room temperature, the reaction was then quenched by the addition of 50 mL of NH₄Cl (aq). The resulting solution was extracted with 3×30 mL of ethyl acetate and the organic layers were combined and concentrated. The residue was purified by combi-flash (EtOAc/PE=1:6) to give the product (1.06 g, 71% yield).

¹H NMR (400 MHZ, CD₃Cl) δ 7.06-7.02 (m, 2 H), 4.08 (s, 2 H), 1.38 (s, 6 H). LCMS (ESI, m/z) [M+1]⁺ 244, 246.
Step 3: 2-chloro-5-(2,2-dimethyl-2,3-dihydro-[1,4]dioxino[2,3-b]pyridin-6-yl)pyridin-4-amine

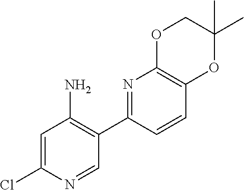 [SMILES: CC1(C)COc2nc(-c3cnc(Cl)cc3N)ccc2O1]

A solution of 2-chloro-5-(4,4,5,5-tetramethyl-1,3,2-dioxaborolan-2-yl)pyridin-4-amine (626.8 mg, 2.46 mmol), 6-bromo-2,2-dimethyl-2,3-dihydro-[1,4]dioxino [2,3-b]pyridine (500 mg, 2.05 mmol), Pd (dppf)Cl₂ (300 mg, 0.41 mmol), K₃PO₄ (869.2 mg, 4.1 mmol) in dioxane (8 mL) and H₂O (2 mL) was stirred at 75° C. for 2 hours. The mixture was cooled to rt and extracted between EA and H₂O. The organic layer was concentrated. The crude product was purified by silica gel column chromatography (PE/EA=1:1) to give the desired product (440 mg, 73.57%). MS (ESI) m/e [M+1] 292.
Step 4: N-(4-amino-5-(2,2-dimethyl-2,3-dihydro-[1,4]dioxino[2,3-b]pyridin-6-yl)pyridin-2-yl)acetamide

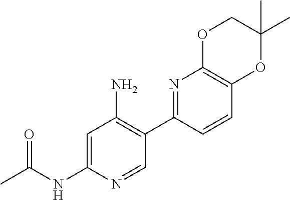 [SMILES: CC(=O)Nc1cc(N)c(-c2ccc3c(n2)OCC(C)(C)O3)cn1]

A solution of 2-chloro-5-(2,2-dimethyl-2,3-dihydro-[1,4]dioxino[2,3-b]pyridin-6-yl)pyridin-4-amine (440 mg, 1.51 mmol), acetamide (445.4 mg, 7.54 mmol), Pd₂(dba)₃ (276.6 mg, 0.3 mmol), Xant-phos (349.5 mg, 0.6 mmol) and Cs₂CO₃ (984.5 mg, 3 mmol) in dioxane (20 mL) was stirred at 130° C. for 5 hours. The mixture was cooled to r.t and filtered through celite. The filtrate was concentrated under reduced pressure. The crude product was purified by silica gel column chromatography (DCM/MeOH=20:1) to give the desired product (305 mg, 64.26%). MS (ESI) m/e [M+1]⁺ 315.
Step 5: N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2- yl)amino)pyridin-2-yl)acetamide (Compound 1)

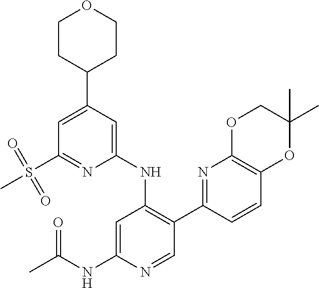

A solution of N-(4-amino-5-(2,2-dimethyl-2,3-dihydro-[1,4]dioxino [2,3-b]pyridin-6-yl)pyridin-2-yl)acetamide (60 mg, 0.2 mmol), 2-bromo-6-(methylsulfonyl)-4-(tetrahydro-2H-pyran-4-yl)pyridine (96 mg, 0.3 mmol), Pd₂(dba)₃ (36.6 mg, 0.04 mmol), Xantphos (46.3 mg, 0.08 mmol) and Cs₂CO₃ (130.4 mg, 0.4 mmol) in dioxane (3 mL) was stirred at 130°° C. for 5 hr. The mixture was cooled to RT and the solid was removed by filtration. The filtrate was concentrated under reduced pressure. The crude product was purified by silica gel column chromatography (DCM/MeOH=15/1) to give the product (63.6 mg, 57%). ¹H NMR (400 MHZ, DMSO-d₆) δ 11.86 (s, 1 H), 10.49 (s, 1 H), 9.06 (s, 1 H), 8.62 (s, 1 H), 7.59 (d, J=8.5 Hz, 1 H), 7.52-7.38 (m, 2 H), 7.05 (s, 1 H), 4.26 (s, 2 H), 4.01-3.92 (m, 2 H), 3.49-3.44 (m, 2 H), 3.43 (s, 3 H), 3.02-2.89 (m, 1 H), 2.11 (s, 3 H), 1.84-1.74 (m, 2 H), 1.72-1.59 (m, 2 H), 1.37 (s, 6 H). MS (ESI) m/e [M+1]⁺ 554.

### Method B:

Step 1: Synthesis of 2-chloro-5-(4,4,5,5-tetramethyl-1,3,2-dioxaborolan-2-yl)pyridin-4-amine

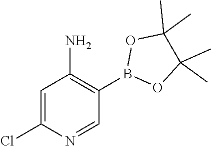 [SMILES: CC1(C)OB(c2cnc(Cl)cc2N)OC1(C)C]

To a reactor were charged 5-bromo-2-chloropyridin-4-amine (22.65 Kg), B₂Pin₂ (33.6 Kg), KOAc (21.4 Kg), and toluene (186.0 Kg), the resulting mixture was swapped with N₂ for three times, then palladium acetate (0.48 Kg) and butyldi-1-adamantylphosphine (0.14 Kg) were charged successively and the resulting mixture was stirred at 85-94° C. for 16-24 h under N₂ protection. Upon completion of the reaction, the mixture was cooled to 25° C., then EtOH (74.0 Kg) and water (120.0 Kg) were charged into the reactor and stirred at this temperature for 1 h. Then the organic later was separated and the mother liquor was extracted with EA (64.0 Kg×2). The combined organic layers were filtered, then crystal seeds (1.65 kg) were charged and the mixture was stirred at this temperature for 0.5-2 h. The solid was collected by filtration and the filter cake was washed with MeOH (20.0 Kg) and dried at 40-50° C. for 24 h. The desired product was isolated with 77.9% yield.
Step 2: synthesis of 2-chloro-5-(2,2-dimethyl-2,3-dihydro-[1,4]dioxino [2,3-b]pyridin-6-yl) pyridin-4-amine

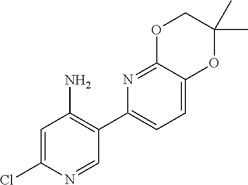 [SMILES: CC1(C)COc2nc(-c3cnc(Cl)cc3N)ccc2O1]

To a reactor were charged water (46.0 Kg), 2-chloro-5-(4,4,5,5-tetramethyl-1,3,2-dioxaborolan-2-yl)pyridin-4-amine (22.2 Kg), 6-bromo-2,2-dimethyl-2,3-dihydro-[1,4]dioxino [2,3-b]pyridine (24.6 Kg), K₃PO₄ (36.8 kg) and isopropanol (140.0 kg), the resulting mixture was swapped with N₂ for three times. Then palladium acetate (0.058 kg) and PCy₃·HBF₄ (0.190 kg) were charged successively and the and the resulting mixture was stirred at 60-70° C. for 16-24 h under N₂ protection. Upon completion of the reaction, the reaction mixture was cooled to 40-50° C. and the water layer was separated. To the organic layer was charged crystal seed (0.55 kg) and aged for 6-10 h. The solid was collected by filtration and the filter cake was washed with water (66.0 Kg) and dried at 40-50° C. for 15-25 h. The desired product was isolated with 85.2% yield.
Step 3: synthesis of N-(2-chloro-5-(2,2-dimethyl-2,3-dihydro-[1,4]dioxino[2,3-b]pyridin-6-yl)pyridin-4-yl)-6-(methylsulfonyl)-4-(tetrahydro-2H- pyran-4-yl)pyridin-2-amine

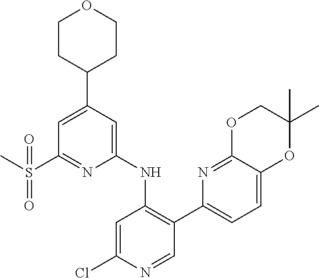

To a reactor were charged 2-Me THF (184.0 Kg), 2-chloro-5-(2,2-dimethyl-2,3-dihydro-[1,4]dioxino[2,3-b]pyridin-6-yl)pyridin-4-amine (21.9 Kg), 2-chloro-6-(methylsulfonyl)-4-(tetrahydro-2H-pyran-4-yl)pyridine (21.7 Kg), K₃PO₄ (31.5 kg) and water (20.0 Kg). The resulting mixture was swapped with N₂ three times and then Pd₂(dba)₃ (0.73 kg) and Xantphos (0.90 kg) were charged. The mixture was stirred at 70-80° C. for 24-34 h under N₂ protection. Upon completion of the reaction, acetonitrile (44.0 Kg) was charged at 30-40° C. and the resulting mixture was stirred at this temperature for 4 h. Then the solid was collected by filtration, then dried at 45-55° C. for 15 h. The desired product was isolated with 76% yield.
Step 4: synthesis of N-(5-(2,2-dimethyl-2,3-dihydro-[1,4]dioxino [2,3-b]pyridin-6-yl)-4-((6-(methylsulfonyl)-4-(tetrahydro-2H-pyran-4-yl)pyridin- 2-yl)amino)pyridin-2-yl)acetamide

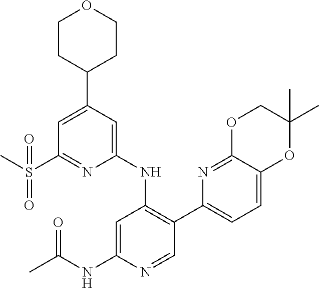

To a reactor were charged 1,4-dioxane (230.0 kg), N-(2-chloro-5-(2,2-dimethyl-2,3-dihydro-[1,4]dioxino[2,3-b]pyridin-6-yl)pyridin-4-yl)-6- (methylsulfonyl)-4-(tetrahydro-2H-pyran-4-yl)pyridin-2-amine (20.0 Kg), Acetamide (3.5 Kg) and K₃PO₄ (16.0 Kg). The resulting mixture was swapped with N₂ three times and then allylpalladium (II) chloride dimer (0.152 kg) and dCypf (0.50 Kg) were added and the mixture was stirred at 95-105° C. for 12 h. After cooled to 20-30° C., acetic acid (4.1 Kg) and water (600.0 Kg) were charged and stirred at 20-30° C. for 3 h. The product was isolated by filtration. The crude product was washed with water (361.0 Kg) and dissolved into NMP (313 Kg) at 45-55° C. To the filtrate was charged water (320 Kg) and stirred at 20-30° C. for 2 h. The crude product was isolated by filtration and the filter cake was washed with water dried at 45-55° C. for 8-12 h. The desired product was isolated in 56% yield.

### Example 2A: Preparation of Compound 1 Form A

### Method A

Compound 1 (30 g) was added into N-methylpyrrolidone (NMP, 300 mL) and then the resulting mixture was heated to reflux to obtain a solution. To the solution was added acetone (300 mL) and the mixture was slowly cooled down to room temperature (RT) and stirred, then the solid was collected by filtration, and the filter cake was washed with acetone and dried over reduced pressure to yield the desired product (25.5 g).

The X-ray powder diffraction (XRPD) pattern (performed with PANalytical X Pert3 XRPD on a Si single crystal holder, K-Alpha1 [Å]: 1.54060 m, K-Alpha2 [Å]: 1.54443, 40 mA, 45 kV) was used to characterize the obtained product, which showed that the product was in a crystalline Form designated as Compound 1 Form A, see FIG. 1A. The peaks and percent peak intensities obtained from the XRPD analysis are listed in Table 1A.

TABLE 1A
XRPD pattern of Compound 1 Form A

| Pos. [°2θ] | d-spacing [Å] | Rel. Int. [%] |
|---|---|---|
| 5.45 | 16.23 | 6.2 |
| 7.50 | 11.78 | 10.8 |
| 10.34 | 8.55 | 11.5 |
| 10.84 | 8.16 | 23.5 |
| 14.80 | 5.98 | 90.6 |
| 15.39 | 5.76 | 26.5 |
| 16.15 | 5.49 | 100.0 |
| 17.49 | 5.07 | 2.3 |
| 17.80 | 4.98 | 3.6 |
| 18.05 | 4.91 | 1.8 |
| 19.75 | 4.50 | 11.0 |
| 20.37 | 4.36 | 8.8 |
| 20.66 | 4.30 | 15.0 |
| 21.56 | 4.12 | 72.3 |
| 22.84 | 3.89 | 5.7 |
| 24.02 | 3.70 | 1.8 |
| 25.04 | 3.56 | 7.4 |
| 25.58 | 3.48 | 25.4 |
| 25.90 | 3.44 | 11.6 |

¹H NMR spectrum of Compound 1 Form A was shown in FIG. 1C. DSC and TGA curves showed that a weight loss of 2.3% to 150° C. and one endothermic peak at 305° C. (peak) was observed (FIG. 1B). The results showed that Compound 1 Form A is an anhydrate.

### Method B

Compound 1 (30 g) was added into Dimethylacetamide (DMAC,300 mL) and the resulting mixture was heated to reflux to obtain a solution. Then to the solution was added butanone (1000 mL) and the mixture was slowly cooled down to room temperature (RT) and stirred, then the solid was collected by filtration, and the filter cake was washed with butanone and dried over reduced pressure to yield the desired product (22.5 g).

### Example 2B: Preparation of Compound 1 Form B

Compound 1 Form A (50-100 mg) was equilibrated in trifluoroethanol (TFE,0.3-0.6mL) at 50° C. for 1 week with stirring, and then filtered by centrifugation, to yield the desired product.

The XRPD pattern was used to characterize the obtained product, which showed that the product was a crystalline designated as Compound 1 Form B, see FIG. 1D. The peaks and percent peak intensities obtained from the XRPD analysis are listed in Table 1B.

TABLE 1B
XRPD pattern of Compound 1 Form B

| Pos. [°2θ] | d-spacing [Å] | Rel. Int. [%] |
|---|---|---|
| 4.93 | 17.92 | 3.0 |
| 6.35 | 13.91 | 100.0 |
| 7.37 | 11.98 | 39.8 |
| 9.77 | 9.04 | 2.7 |
| 12.28 | 7.20 | 0.8 |
| 12.68 | 6.98 | 3.1 |
| 13.24 | 6.68 | 6.1 |
| 14.74 | 6.01 | 0.8 |
| 16.25 | 5.45 | 2.3 |
| 17.68 | 5.01 | 5.3 |
| 18.13 | 4.89 | 2.1 |
| 19.04 | 4.66 | 2.3 |
| 19.56 | 4.54 | 3.1 |
| 20.41 | 4.35 | 1.4 |
| 20.54 | 4.32 | 1.4 |
| 21.02 | 4.22 | 5.3 |
| 21.79 | 4.08 | 3.4 |
| 22.55 | 3.94 | 1.4 |
| 22.70 | 3.91 | 2.0 |
| 24.33 | 3.66 | 2.4 |

KF showed that Compound 1 Form B contained about 3.1% water by weight (1.0 equivalent by molar ratio). ¹H-NMR showed about 7.1% TFE by weight (0.4 equivalent by molar ratio, 0.4 equivalent by molar ratio, FIG. 1G). TGA showed about 3.0% weight loss from 35° C. to 80°° C., about 3.3% weight loss from 80°° C. to 150°° C., and about 3.0% weight loss from 150°° C. to 250° C. (FIG. 1E). DSC showed that multiple thermal events (FIG. 1F). The results showed that Compound 1 Form B was a TFE and water hetero-solvate.

### Example 2C: Preparation of Compound 1 Form C

Compound 1 Form A (50-100 mg) was equilibrated in 0.3-0.7 mL of 1,4-dioxane at 25° C. for 2weeks with stirring and then collected by centrifugation, to obtain the desired product.

The XRPD pattern was used to characterize the obtained product which showed that the product was in a crystalline designated as Compound 1 Form C, see FIG. 1H. The peaks and percent peak intensities obtained from the XRPD analysis are listed in Table 1C.

TABLE 1C
XRPD pattern of Compound 1 Form C

| Pos. [°2θ] | d-spacing [Å] | Rel. Int. [%] |
|---|---|---|
| 7.21 | 12.26 | 11.1 |
| 7.44 | 11.88 | 29.0 |
| 7.78 | 11.35 | 21.0 |
| 10.29 | 8.59 | 19.4 |
| 10.79 | 8.19 | 60.6 |
| 13.35 | 6.63 | 6.5 |
| 14.39 | 6.15 | 10.4 |
| 14.76 | 6.00 | 45.0 |
| 15.33 | 5.78 | 7.6 |
| 15.67 | 5.65 | 10.0 |
| 16.10 | 5.50 | 17.7 |
| 16.69 | 5.31 | 30.8 |
| 16.87 | 5.25 | 33.9 |
| 17.44 | 5.08 | 51.3 |
| 17.73 | 5.00 | 69.1 |
| 20.02 | 4.43 | 13.3 |
| 20.70 | 4.29 | 80.5 |
| 21.26 | 4.18 | 17.3 |
| 21.58 | 4.11 | 78.6 |
| 22.55 | 3.94 | 27.4 |
| 22.78 | 3.90 | 95.8 |
| 23.97 | 3.71 | 13.4 |
| 24.96 | 3.56 | 11.1 |

KF showed about 0.03% water by weight (0.01 equivalent by molar ratio). ¹H-NMR showed about 9.5% 1,4-dioxane by weight (0.7 equivalent by molar ratio) (FIG. 1K). TGA showed about 10.2% weight loss from 37° C. to 120° C., and about 4.2% weight loss from 120° C. to 200° C. (FIG. 1I). DSC showed an endothermic peak at 85.9° C. (Tonset), followed by an endothermic peak at 137.9° C., and a melting peak at 302.9° C. (FIG. 1J), which suggested that Compound 1 Form C converted to Compound 1 Form A after desolvation. The results showed that Compound 1 Form C is a 1,4-dioxane solvate.

### Example 2D: Preparation of Compound 1 Form D

Compound 1 Form A (50-100 mg) was equilibrated in 0.3-0.7 mL of chloroform at 25° C. for 2 weeks with stirring and then collected by centrifugation, to obtain the desired product.

The XRPD pattern was used to characterize the obtained product which showed that the product was in a crystalline designated as Compound 1 Form D, see FIG. 1L. The characteristic peaks and percent peak intensities obtained from the XRPD analysis are listed in Table 1D.

TABLE 1D
XRPD pattern of Compound 1 Form D

| Pos. [°2θ] | d-spacing [Å] | Rel. Int. [%] |
|---|---|---|
| 9.37 | 9.43 | 31.2 |
| 9.75 | 9.06 | 21.5 |
| 11.81 | 7.49 | 9.7 |
| 16.10 | 5.50 | 42.1 |
| 16.52 | 5.36 | 46.9 |
| 18.13 | 4.89 | 37.2 |
| 18.76 | 4.73 | 94.0 |
| 19.22 | 4.61 | 51.4 |
| 19.63 | 4.52 | 100.0 |
| 20.10 | 4.41 | 62.3 |
| 21.61 | 4.11 | 58.4 |
| 22.66 | 3.92 | 25.9 |
| 24.19 | 3.68 | 33.7 |

¹H-NMR showed about 19.9% chloroform by weight (1.2 equivalent by molar ratio) (FIG. 1O). TGA showed about 20.4% weight loss at about 150° C. (FIG. 1M). DSC showed an endothermic peak at about 105° C. (Tonset), followed by a melting peak at about 303° C. (FIG. 1N). Compound 1 Form D might convert to Form A after desolvation, compared to the DSC curve of Compound 1 Form A. The results showed that Compound 1 Form D is a chloroform solvate.

### Example 2E: Preparation of Compound 1 Form E

400 mg of Compound 1 Hydrochloride Form A (prepared by the method of Example 3A in this application), was weighed into a 20 mL glass vial. 4 mL of water was added into the vial under stirring at 37° C. 86 mg of NaHCO₃ (1.5 equivalents by molar ratio) was dissolved in 2 mL of water. Then the clear solution was added into Hydrochloride Form A suspension for about 1 min. After stirring at 37° C. for 30 min, solids were collected by suction filtration and washed with water three times, and then dried at ambient conditions for about 16 h. Compound 1 Form E was obtained as an off-white solid.

The XRPD pattern was used to characterize the obtained product which showed that the product was in a crystalline designated as Compound 1 Form E, see FIG. 10A. The characteristic peaks and percent peak intensities obtained from the XRPD analysis are listed in Table 1E.

TABLE 1E
XRPD pattern of Compound 1 Form E

| Pos. [°2θ] | d-spacing [Å] | Rel. Int. [%] |
|---|---|---|
| 4.34 | 20.34 | 56.3 |
| 6.89 | 12.83 | 100.0 |
| 13.10 | 6.75 | 33.8 |
| 13.71 | 6.45 | 23.8 |
| 15.36 | 5.76 | 20.7 |
| 16.53 | 5.36 | 45.7 |
| 18.42 | 4.81 | 92.5 |
| 19.64 | 4.52 | 40.1 |
| 20.69 | 4.29 | 13.0 |
| 21.73 | 4.09 | 17.2 |
| 22.76 | 3.90 | 13.6 |
| 24.70 | 3.60 | 28.6 |
| 25.46 | 3.50 | 36.8 |
| 26.10 | 3.41 | 66.3 |
| 28.49 | 3.13 | 9.6 |

¹H NMR spectrum of Compound 1 Form E was shown in FIG. 10D. DSC and TGA curves showed that a weight loss of 4.3% to 130° C. and one endothermic peak at 303° C. (peak) was observed (FIG. 10C and FIG. 10B). The results showed that Compound 1 Form E is a hydrate.

### Example 2F: Physicochemical Stability of Compound 1 Form A

Compound 1 Form A still kept the same Form in the following experiments, which showed that Compound 1 Form A has good physicochemical stability.
equilibration (25° C.) experiments conducted in 18 solvent systems, selected from water, methanol. ethanol, acetone, ACN, ethyl acetate, THF, 2-MeTHF, DCM, DMSO, DMF, NMP, DMAc, MeOH/water(40:60, v/v), acetone/water (50:50, v/v), ACN/water(90:10, v/v), THF/water (85:15, v/v), and DMSO/water(30:70, v/v);
equilibration (50° C.) experiments conducted in 19 solvent systems, selected from water, methanol, ethanol, acetone, ACN, ethyl acetate, 1,4-dioxane, THF, 2-MeTHF, CHCl3, DMSO, DMF, NMP, DMAc, MeOH/water(40:60, v/v), acetone/water(50:50, v/v), ACN/water(90:10, v/v), THF/water(85:15, v/v) and DMSO/water(30:70, v/v);
slow evaporation experiments conducted in 5 solvent systems, selected from DCM, CHCl3,DCM/MeOH (50:50, v/v), DCM/EtOH (50:50, v/v) and DCM/2-MeTHF (50:50, v/v);
fast evaporation experiments conducted in 4 solvent systems, selected from DMSO, DMF, DCM and DCM/MeOH (50:50, v/v);
anti-solvent experiments conducted in 8 solvent systems, selected from DCM/heptane, DCM/MTBE, DMAc/water, DMAc/MTBE, NMP/water, NMP/MTBE, CHCl₃/heptane CHCl₃3/MTBE; and,
DSC heating-cooling cycle experiments.

### Example 3A: Preparation of Compound 1 Hydrochloride Form A

### Method A:

To a mixture of Compound 1 (10 g) in 300 mL of acetone/H₂O (v/v=9/1) was added about 1.0 equivalent HCl water solution (4 N) under stirring at 50° C,. and the resulting mixture was stirred at this temperature for 2 hours, at 40° C. for 2 hours, then at room temperature for 16 hours. The precipitated solid was collected by filtration and the filter cake was washed with acetone and dried at 50° C. to give the product (9.0 g, Yield: 84.5%).

The X-ray powder diffraction (XRPD) pattern showed that the obtained product was a physical mixture of Compound 1 hydrochloride Form C and Form D, which was highly crystalline designated as Compound 1 hydrochloride Form A, sec FIG. 2A.

### Method B:

Compound 1 Form A (4 g) and Compound 1 hydrochloride Form A seed (about 26 mg) was added into 120 mL of acetone/water (v/v=9/1) with stirring at 50°° C. to obtain a slurry. To the mixture was added HCl acetone/water solution (HCl (12 N, 0.64 mL) in acetone/water (1.28 mL, v/v=9:1)) slowly, followed with Compound 1 hydrochloride Form A seeds (24 mg), and then stirred at 50° C. for about 2 hours, slowly cooled to 40° C., hold at 40° C. for 2 hours, slowly cooled to RT, and hold at RT for about 16 hours, to obtain the desired product (4026 mg).

The X-ray powder diffraction (XRPD) pattern showed that the obtained product was a physical mixture of Compound 1 hydrochloride Form C and Form D, which was highly crystalline designated as Compound 1 hydrochloride Pattern A. TGA showed about 0.9% weight loss at about 194° C. DSC showed no melting peak before decomposition. HPLC and Ion chromatography (IC) showed Compound 1: HCl was 1:1.0 and 1.4% acetone residual by weight, which was detected by ¹H-NMR (FIG. 2B). KF showed that Compound 1 hydrochloride Form A contained about 0.3% water by weight, equivalent to 0.1 water molecules.

### Example 3B: Preparation of Compound 1 Hydrochloride Form B

Compound 1 Form A (50 mg) and 1 equivalent of HCl were added into ACN (acetonitrile), stirred at 50° C. for 2 hours, and then at 25° C. for at least 12 hours, and the solid was collected by filtration to obtain the desired product.

The XRPD pattern was used to characterize the obtained product, which showed that the product was in a crystalline designated as Compound 1 hydrochloride Form B, see FIG. 2C. The peaks and percent peak intensities obtained from the XRPD analysis are listed in Table 2A.

TABLE 2A
XRPD pattern of Compound 1 hydrochloride Form B

| Pos. [°2θ] | d-spacing [Å] | Rel. Int. [%] |
|---|---|---|
| 7.06 | 12.51 | 100.0 |
| 7.31 | 12.08 | 55.3 |
| 7.58 | 11.66 | 51.5 |
| 9.67 | 9.14 | 8.8 |
| 12.44 | 7.11 | 43.0 |
| 13.20 | 6.70 | 41.3 |
| 13.78 | 6.42 | 32.2 |
| 14.14 | 6.26 | 5.8 |
| 14.60 | 6.06 | 28.8 |
| 15.04 | 5.88 | 9.8 |
| 15.48 | 5.72 | 77.6 |
| 15.82 | 5.60 | 71.5 |
| 16.31 | 5.43 | 16.8 |
| 17.19 | 5.16 | 16.0 |
| 18.61 | 4.76 | 14.6 |
| 18.98 | 4.67 | 38.9 |
| 19.23 | 4.61 | 28.6 |
| 19.68 | 4.51 | 30.7 |
| 19.97 | 4.44 | 41.9 |
| 20.98 | 4.23 | 87.7 |
| 21.77 | 4.08 | 43.6 |
| 22.20 | 4.00 | 40.8 |
| 22.92 | 3.88 | 68.0 |
| 24.02 | 3.70 | 32.9 |
| 24.38 | 3.65 | 89.1 |

¹H-NMR showed about 1.2% ACN by weight by weight (the stoichiometric ratio of Compound 1: acid was 1:1.0, FIG. 2F). TGA showed about 3.7% weight loss from about 34 to 118° C., and about 6.7% weight loss from about 118 to 228° C. (FIG. 2D). DSC showed an endothermic peak at 66° C. of 37° C. (Tonset), followed by a melting peak at 236° C. (FIG. 2E).

### Example 3C: Preparation of Compound 1 Hydrochloride Form C

### Method A:

To a mixture of Compound 1 (10.0 g, 18.08 mmol) in 300 mL of MeOH/H₂O (v/v=9/1) was added about 1.0 equivalent HCl (4N) under stirring at about 50° C., the resulting mixture was stirred at about 50° C. for 16 hours, then slowly cooled to room temperature. The precipitated solid was collected by filtration and the filter cake was rinsed with MeOH (20 mL×2) and dried under vacuum at about 50° C. overnight to afford 7.5 g of product (70.3% yield).

### Method B:

A mixture of Compound 1 (10.0 g, 18.08 mmol) in 300 mL of MeOH (30 v) was heated to about 60° C., then HCl/MeOH solution (3 M, 7.22 mL, 21.70 mmol, 1.2 eq) was added dropwise and the resulting mixture was stirred at about 60° C. for 16 hours. After cooled to room temperature, the precipitated solid was collected by filtration, and the filter cake was rinsed with MeOH (20 mL×2), dried under vacuum at 50° C. overnight to afford 8.8 g product (82.3% yield).

### Method C:

To a mixture of Compound 1 (1.0 g, 1.81 mmol) in 30 mL of NMP/MeOH (v/v=1/4) was added HCl/MeOH (3.0 M, 0.72 mL, 1.2 eq) solution in dropwise under stirring at about 50° C., and the resulting mixture was stirred at this temperature for 16 hours. After cooled to room temperature, the precipitated solid was collected by filtration, and the filter cake was rinsed with MeOH (20 mL×2), dried under vacuum at 50° C. overnight to afford 850 mg product (80.1% yield).

### Method D:

Compound 1 (11.92 kg) and water (6× volume) were mixed. After heating to a temperature of about 50° C. 4N HCl aq. (0.24 eq) was added followed by adding the crystal seed of Compound 1 hydrochloride Form C, and stirred for 1.5 hours, to obtain a mixture. The mixture was added with 4N HCl aq in two batches (0.96 eq, 0.3 eq) and then stirred for 9 hours and 2 hours, separately. Acetone (2 volumes) was added and stirred for 2 hours, and then cooled to 10-20° C., the solid was collected by filtration and dried to obtain the desired product (11.72 kg, HPLC purity 99.8%). 1H NMR (400 MHZ, DMSO-d6) δ 12.04 (s, 1 H), 11.53 (s, 1 H), 8.67 (s, 1 H), 8.58 (s, 1 H), 7.61 (s, 1 H), 7.53 (d, J=6.0 Hz, 1 H), 7.49 (d, J=6.0 Hz, 1 H), 7.20 (s, 1 H), 4.26 (s, 2 H), 3.95-3.97 (m, 2 H), 3.44-3.42 (m, 5 H), 3.03-2.97 (m, 1 H), 2.20 (s, 3 H), 1.81-1.80 (m, 2 H), 1.75-1.61 (m, 2 H), 1.36 (s, 6 H).

The XRPD pattern was used to characterize the obtained product, which showed that the product was in a crystalline designated as Compound 1 hydrochloride Form C, see FIG. 2G. The peaks and percent peak intensities obtained from the XRPD analysis are listed in Table 2B.

TABLE 2B
XRPD pattern of Compound 1 hydrochloride Form C

| Pos. [°2θ] | d-spacing [Å] | Rel. Int. [%] |
|---|---|---|
| 6.93 | 12.74 | 92.7 |
| 9.06 | 9.76 | 61.8 |
| 9.69 | 9.12 | 8.9 |
| 10.63 | 8.31 | 3.4 |
| 13.16 | 6.72 | 3.6 |
| 13.81 | 6.41 | 39.5 |
| 14.19 | 6.23 | 10.9 |
| 15.79 | 5.61 | 5.8 |
| 16.19 | 5.47 | 71.8 |
| 17.63 | 5.03 | 4.6 |
| 18.15 | 4.88 | 100.0 |
| 18.59 | 4.77 | 43.4 |
| 19.22 | 4.61 | 24.4 |
| 19.53 | 4.54 | 18.8 |
| 19.86 | 4.47 | 78.3 |
| 20.14 | 4.40 | 24.4 |
| 20.91 | 4.25 | 27.6 |
| 21.33 | 4.16 | 22.6 |
| 22.18 | 4.01 | 7.2 |
| 22.86 | 3.89 | 30.7 |
| 24.02 | 3.70 | 95.6 |
| 24.26 | 3.67 | 44.1 |
| 24.62 | 3.61 | 7.8 |
| 24.85 | 3.58 | 11.7 |

¹H-NMR showed no solvent residue (FIG. 2J). TGA showed no obvious weight loss from 30 to 150° C. (FIG. 2H). DSC showed no thermal transition event from 30° C. to 200° C. and started to dissociate at about 200° C. (FIG. 2I). TGA data were collected using a Discovery 550; and DSC was performed using a TA Discovery 250 Instrument.

### Example 3D: Preparation of Compound 1 Hydrochloride Form D

Compound 1 hydrochloride Pattern A (500 mg) and ACN/water (90/10, v/v, 4.0 mL) were mixed and stirred at RT. After being stirred for about 20 days at RT, and at 50° C. for about 1 day, the solids in the mixture stirred were collected by filtration and then dried, to obtain the desired product.

The XRPD pattern was used to characterize the obtained product, which showed that the product was in a crystalline designated as Compound 1 hydrochloride Form D, see FIG. 2K. The peaks and percent peak intensities obtained from the XRPD analysis are listed in Table 2C.

TABLE 2C
XRPD pattern of Compound 1 hydrochloride Form D

| Pos. [°2θ] | d-spacing [Å] | Rel. Int. [%] |
|---|---|---|
| 7.20 | 12.26 | 100.0 |
| 8.91 | 9.92 | 14.2 |
| 9.94 | 8.89 | 12.3 |
| 13.23 | 6.69 | 44.5 |
| 14.38 | 6.15 | 42.2 |
| 16.34 | 5.42 | 23.1 |
| 16.55 | 5.35 | 18.4 |
| 17.00 | 5.21 | 5.4 |
| 17.46 | 5.07 | 27.7 |
| 17.63 | 5.03 | 18.1 |
| 19.15 | 4.63 | 30.1 |
| 19.76 | 4.49 | 11.0 |
| 20.13 | 4.41 | 19.0 |
| 20.45 | 4.34 | 61.3 |
| 20.75 | 4.28 | 8.4 |
| 21.22 | 4.18 | 7.9 |
| 21.62 | 4.11 | 20.6 |
| 22.65 | 3.92 | 4.6 |
| 23.47 | 3.79 | 50.0 |
| 24.37 | 3.65 | 20.5 |
| 24.94 | 3.57 | 11.7 |

1H-NMR showed about 0.07% ACN by weight (0.01 equivalent by molar ratio, FIG. 2N). TGA showed about 0.6% weight loss from about 35 to 170° C. (FIG. 2L). DSC showed an endothermic peak at 176° C. of 174° C. (Tonset) (FIG. 2M).

### Example 3E: Preparation of Compound 1 Hydrochloride Form C and Form D in Single Crystal Compound 1 Hydrochloride Form C in Single Crystal

Compound 1 hydrochloride Form C (100 mg) and acetonitrile/water (90/10, v/v, 5 mL) were mixed. The obtained mixture was stirred at 50° C. for 10 min, and then stirred at 50° C. for 10 min and filtered by a syringe membrane filter, to obtain a clear solution. The solution was divided and transferred to a vial and equilibrated under a temperature cycle between 5° C. to 50° C. at a heating/cooling rate of 0.1° C./min for about 20 cycles. After temperature cycles, the sample was placed at ambient conditions (15-25° C., 50-80 RH) for about 9 days, to obtain needle-like crystals and plate crystals.

Then the plate crystal was determined to be Compound 1 hydrochloride Form D, and the needle-like crystal was determined to be Compound 1 hydrochloride Form C, the Compound 1 hydrochloride Form C crystallized in a monoclinic system with P21/c space group. The crystallographic data were collected 298 K on a Single Crystal X-ray Diffractometer (SCXRD), Bruker D8 Venture with CMOS area detector (Radiation was Mo/K-Alpha1 (λ=0.71073 Å); X-ray generator power was 50 kV, 1.4 mA). No solvent molecule was contained. The simulated XRPD pattern based on the single-crystal data at 298(2)K (FIG. 2O) is in accordance with XRPD of Compound 1 hydrochloride Form C.

TABLE 2D
Crystal Data of Compound 1 Hydrochloride Form C in Single Crystal

| | |
|---|---|
| Monoclinic, P2₁/c | F(000) = 1240 |
| a = 9.846 (5) Å | Dx = 1.415 Mg m⁻³ |
| b = 25.660 (11) Å | Mo Ka radiation, λ = 0.71073 Å |
| c = 10.975 (4) Å | Cell parameters from 1965 reflections |
| β = 92.551 (15)° | theta = 2.2-25.1° |
| V = 2770 (2) Å3 | u= 0.27 mm⁻¹ |
| Z = 4 | T = 298K |
| | Needle, colourless |
| | 0.16 × 0.04 × 0.02 mm |

### Compound 1 Hydrochloride Form D in Single Crystal

Compound 1 Form C (20 mg) and acetonitrile/water (80:20, v/v) were mixed. The obtained mixture was subjected to sonication for about 30 sec, heated at 50° C. for about 10 min, filtered by a syringe membrane filter to obtain a clear solution. The solution was to allow slow evaporation of solvents under ambient conditions (15-25° C., 50-80 RH), to obtain plate single crystals.

The crystal structure was determined with the obtained plate crystals by evaporation. This Compound 1 hydrochloride crystal was crystallized in a monoclinic system with P21/n space group. The crystallographic data were collected at 298 K on a Single Crystal X-ray Diffractometer (SCXRD), Bruker D8 Venture with a CMOS area detector (Radiation was Mo/K-Alpha1 (λ=0.71073 Å); X-ray generator power was 50 kV, 1.4 mA). No solvent molecule was contained.

The simulated XRPD pattern based on the single-crystal data at 298 K (FIG. 2P) is in accordance with XRPD of Compound 1 hydrochloride Form D.

TABLE 2E
Crystal Data of Compound 1 Hydrochloride Form D in Single Crystal

| | |
|---|---|
| Monoclinic, P2₁/n | F(000) = 1240 |
| a = 10.309 (9) Å | Dx = 1.418 Mg m⁻³ |
| b = 10.855 (7) Å | Mo Ka radiation, λ = 0.71073 Å |
| c = 24.995 (17) Å | Cell parameters from 2063 reflections |
| β = 98.84 (3)° | theta = 2.3-25.7° |
| V = 2764 (4) Å3 | u = 0.27 mm⁻¹ |
| Z = 4 | T = 298K |
| | Block, colourless |
| | 0.15 × 0.08 × 0.05 mm |

### Example 4A: Preparation of Compound 1 Sulfate Form A

About 50 mg of Compound 1 Form A and 1 equiv. of H₂SO₄ (sulfuric acid) were added into acetone, stirred at 50° C. for 2 hours and then at 25° C. for at least 12 hours, and filtered to obtain the desired product.

The XRPD pattern was used to characterize the obtained product, which showed that the product was in a crystalline designated as Compound 1 sulfate Form A, see FIG. 3A. The peaks and percent peak intensities obtained from the XRPD analysis are listed in Table 3A.

TABLE 3A
XRPD pattern of Compound 1 sulfate Form A

| Pos. [°2θ] | d-spacing [Å] | Rel. Int. [%] |
|---|---|---|
| 5.96 | 14.83 | 48.4 |
| 6.98 | 12.65 | 29.8 |
| 9.71 | 9.10 | 5.7 |
| 10.46 | 8.45 | 5.0 |
| 11.35 | 7.79 | 10.9 |
| 11.95 | 7.40 | 10.8 |
| 13.01 | 6.80 | 17.6 |
| 13.96 | 6.34 | 13.4 |
| 16.18 | 5.48 | 92.2 |
| 16.78 | 5.28 | 26.8 |
| 17.42 | 5.09 | 89.8 |
| 18.12 | 4.89 | 32.5 |
| 18.72 | 4.74 | 55.6 |
| 19.13 | 4.64 | 95.4 |
| 20.54 | 4.32 | 22.3 |
| 21.64 | 4.10 | 7.0 |
| 22.16 | 4.01 | 46.7 |
| 22.66 | 3.92 | 22.2 |
| 23.33 | 3.81 | 66.6 |
| 23.78 | 3.74 | 74.6 |
| 24.39 | 3.65 | 21.2 |
| 24.72 | 3.60 | 100.0 |

¹H-NMR showed about 0.2% acetone by weight (0.02 equivalent by molar ratio, the stoichiometric ratio of Compound 1: acid was 1:1.2, FIG. 3D). TGA showed about 1.7% weight loss from about 34 to 129° C. (FIG. 3B). DSC showed desolvation at 33° C. (Tonset), followed by a melting peak at 226° C. (FIG. 3C).

### Example 4B: Preparation of Compound 1 Sulfate Form B

Compound 1 Form A (50 mg) and 1 equiv. of H₂SO₄ were added into acetonitrile (ACN), stirred at 50° C. for 2 hours and then at 25° C. for at least 12 hours, and filtered to yield Compound 1 sulfate Form B.

The XRPD pattern was used to characterize the obtained product, which showed that the product was in a crystalline designated as Compound 1 sulfate Form B, see FIG. 3E. The peaks and percent peak intensities obtained from the XRPD analysis are listed in Table 3B.

TABLE 3B
XRPD pattern of Compound 1 sulfate Form B

| Pos. [°2θ] | d-spacing [Å] | Rel. Int. [%] |
|---|---|---|
| 5.92 | 14.92 | 93.7 |
| 6.75 | 13.08 | 58.1 |
| 11.47 | 7.71 | 42.5 |
| 11.93 | 7.41 | 19.5 |
| 12.37 | 7.15 | 29.1 |
| 12.62 | 7.01 | 12.9 |
| 13.46 | 6.57 | 30.9 |
| 14.81 | 5.98 | 13.5 |
| 16.04 | 5.52 | 30.1 |
| 16.34 | 5.42 | 58.8 |
| 17.18 | 5.16 | 43.8 |
| 17.72 | 5.00 | 48.9 |
| 18.68 | 4.75 | 9.7 |
| 19.26 | 4.61 | 100.0 |
| 19.62 | 4.52 | 40.4 |
| 20.45 | 4.34 | 9.8 |
| 21.11 | 4.21 | 43.7 |
| 22.19 | 4.00 | 11.7 |
| 22.67 | 3.92 | 16.5 |
| 23.36 | 3.80 | 44.4 |
| 23.60 | 3.77 | 35.7 |
| 24.64 | 3.61 | 79.1 |

¹H-NMR showed about 0.8% ACN by weight (the stoichiometric ratio of Compound 1: acid was 1:1.2, FIG. 3H). TGA showed about 2.3% weight loss from about 34 to 125° C. (FIG. 3F). DSC showed an endothermic peak at 45° C. (Tonset=33° C.), followed by an onset of 238° C. and a melting peak at 243° C. (FIG. 3G).

### Example 5A: Preparation of Compound 1 Phosphate Form A

### Method A:

Compound 1 Form A (800 mg) was added into ACN (6.4 mL) and stirred at 50° C. to obtain a suspension. To the suspension was added phosphoric acid solution (0.2 mL of 85% water solution, diluted with 1.8 mL of ACN, about 2.05 equivalent by molar ratio) slowly, then Compound 1 phosphate Form A seeds (5 mg) was added and stirred at 50° C. for about 2 hours. Additional ACN (5 mL) was added to maintain suspension and kept stirring for about 1 day. The solid was collected by filtration and the filter cake was dried to obtain the desired product.

HPLC and IC showed that the stoichiometric ratio of Compound 1:phosphoric acid was about 1:2.2.

The XRPD pattern was used to characterize the obtained product, which showed that the product was in a crystalline designated as Compound 1 phosphate Form A, see FIG. 4A. The peaks and percent peak intensities obtained from the XRPD analysis are listed in Table 4A.

TABLE 4A
XRPD pattern of Compound 1 phosphate Form A

| Pos. [°2θ] | d-spacing [Å] | Rel. Int. [%] |
|---|---|---|
| 6.95 | 12.70 | 41.5 |
| 9.02 | 9.79 | 2.9 |
| 11.06 | 8.00 | 26.0 |
| 12.18 | 7.26 | 6.8 |
| 12.62 | 7.01 | 39.9 |
| 13.18 | 6.71 | 32.1 |
| 13.86 | 6.39 | 14.1 |
| 14.51 | 6.10 | 52.1 |
| 15.83 | 5.59 | 10.3 |
| 16.81 | 5.27 | 26.9 |
| 17.60 | 5.03 | 12.3 |
| 18.10 | 4.90 | 31.0 |
| 18.57 | 4.77 | 23.9 |
| 19.42 | 4.57 | 14.9 |
| 19.76 | 4.49 | 61.4 |
| 20.68 | 4.29 | 35.8 |
| 20.91 | 4.25 | 27.8 |
| 21.69 | 4.09 | 26.6 |
| 22.23 | 4.00 | 19.9 |
| 22.91 | 3.88 | 19.0 |
| 23.31 | 3.81 | 16.7 |
| 24.11 | 3.69 | 100.0 |
| 24.63 | 3.61 | 10.5 |

¹H-NMR showed 0.5% ACN by weight (0.10 equivalent by molar ratio, FIG. 4D). TGA showed about 0.9% weight loss from about 34 to about 180° C. (FIG. 4B). DSC showed a decomposition upon melting peak having an onset of 234° C. (FIG. 4C). Karl Fisher showed 0.2% water by weight (0.08 equivalent by molar ratio). The results showed that Compound 1 phosphate Form A is an anhydrate.

### Method B:

Compound 1 Form A (50 mg) and 1 equiv. of H₃PO₄ (phosphoric acid) were added into acetone, stirred at 50° C. for 2 hours and then at 25° C. for at least 12 hours, and filtered to obtain the desired product.

### Example 6A: Preparation of Compound 1 Maleate Form A

Compound 1 Form A (50 mg) and 1 equiv. of maleic acid was added into acetone, stirred at 50° C. for 2 hours and then at 25° C. for at least 12 hours, and filtered to obtain the desired product.

The XRPD pattern was used to characterize the obtained product, which showed that the product was in a crystalline designated as Compound 1 maleate Form A, see FIG. 5A. The peaks and percent peak intensities obtained from the XRPD analysis are listed in Table 5A.

TABLE 5A
XRPD pattern of Compound 1 Maleate Form A

| Pos. [°2θ] | d-spacing [Å] | Rel. Int. [%] |
|---|---|---|
| 5.64 | 15.67 | 59.2 |
| 6.97 | 12.67 | 17.9 |
| 11.82 | 7.48 | 13.5 |
| 14.47 | 6.12 | 5.6 |
| 14.95 | 5.92 | 8.1 |
| 15.75 | 5.62 | 23.3 |
| 16.41 | 5.40 | 100.0 |
| 16.76 | 5.29 | 12.4 |
| 17.42 | 5.09 | 19.8 |
| 17.90 | 4.95 | 16.0 |
| 18.17 | 4.88 | 46.0 |
| 18.81 | 4.71 | 22.3 |
| 19.06 | 4.65 | 59.6 |
| 19.77 | 4.49 | 5.2 |
| 20.82 | 4.26 | 46.6 |
| 21.15 | 4.20 | 11.6 |
| 21.53 | 4.12 | 13.0 |
| 21.77 | 4.08 | 18.5 |
| 22.40 | 3.97 | 6.2 |
| 22.90 | 3.88 | 24.1 |
| 23.86 | 3.73 | 46.6 |
| 24.13 | 3.69 | 68.0 |

¹H-NMR showed no residual solvent (the stoichiometric ratio of Compound 1: acid was 1:1.0, FIG. 5D). TGA showed about 0.4% weight loss from about 34 to 150° C. (FIG. 5B). DSC showed an endothermic peak having an onset of about 205° C. and a melting peak at about 215° C. (FIG. 5C). All the results showed that Compound 1 maleate salt Form A is an anhydrate.

### Example 7A: Preparation of Compound 1 Fumarate Form A

Compound 1 Form A (50 mg) and 1 equiv. of fumaric acid was added into 2,2,2-Trifluoroethanol (TFE), stirred at 50° C. for 2 hours and then at 25° C. for at least 12 hours, and filtered to obtain the desired product.

The XRPD pattern was used to characterize the obtained product, which showed that the product was in a crystalline designated as Compound 1 fumarate Form A, see FIG. 6A. The peaks and percent peak intensities obtained from the XRPD analysis are listed in Table 6A.

TABLE 6A
XRPD pattern of Compound 1 fumarate Form A

| Pos. [°2θ] | d-spacing [Å] | Rel. Int. [%] |
|---|---|---|
| 4.66 | 18.94 | 11.3 |
| 5.71 | 15.48 | 81.2 |
| 6.40 | 13.79 | 56.2 |
| 7.59 | 11.64 | 5.6 |
| 10.27 | 8.61 | 6.1 |
| 11.38 | 7.77 | 5.4 |
| 11.80 | 7.50 | 4.2 |
| 13.24 | 6.68 | 10.5 |
| 14.00 | 6.32 | 14.0 |
| 14.50 | 6.11 | 12.4 |
| 15.93 | 5.56 | 13.1 |
| 16.14 | 5.49 | 22.0 |
| 16.52 | 5.36 | 12.5 |
| 17.39 | 5.10 | 16.6 |
| 18.15 | 4.88 | 14.3 |
| 18.66 | 4.75 | 53.0 |
| 19.19 | 4.62 | 8.4 |
| 19.81 | 4.48 | 15.0 |
| 20.50 | 4.33 | 12.4 |
| 22.27 | 3.99 | 12.2 |
| 22.84 | 3.89 | 28.6 |
| 23.47 | 3.79 | 8.9 |
| 24.18 | 3.68 | 6.1 |

¹H-NMR showed no residual solvent (the stoichiometric ratio of Compound 1: acid was 1:1.2, FIG. 6D). TGA showed about 2.2% weight loss from about 34 to 117° C. (FIG. 6B). DSC showed an endothermic peak at 51° C. (Tonset=32° C.), followed by an onset of 221° C. and a melting peak at about 235° C. (FIG. 6C).

### Example 7B: Preparation of Compound 1 Fumarate Form B

### Method A

Compound 1 Form A (50 mg) and 1 equiv. of fumaric acid were added into acetone, stirred at 50° C. for 2 hours and then at 25° C. for at least 12 hours, and filtered to obtain the desired product.

The XRPD pattern was used to characterize the obtained product, which showed that the product was in a crystalline designated as Compound 1 fumarate Form B, see FIG. 6E. The peaks and percent peak intensities obtained from the XRPD analysis are listed in Table 6B.

TABLE 6B
XRPD pattern of Compound 1 fumarate Form B.

| Pos. [°2θ] | d-spacing [Å] | Rel. Int. [%] |
|---|---|---|
| 6.93 | 12.75 | 50.8 |
| 10.10 | 8.75 | 6.1 |
| 12.19 | 7.25 | 19.7 |
| 13.95 | 6.34 | 22.8 |
| 15.09 | 5.87 | 29.3 |
| 15.50 | 5.71 | 15.4 |
| 16.09 | 5.50 | 15.9 |
| 17.06 | 5.19 | 25.0 |
| 17.24 | 5.14 | 34.9 |
| 19.55 | 4.54 | 19.7 |
| 19.77 | 4.49 | 14.6 |
| 20.40 | 4.35 | 69.8 |
| 21.33 | 4.16 | 13.2 |
| 21.84 | 4.07 | 31.2 |
| 22.95 | 3.87 | 15.9 |
| 23.27 | 3.82 | 15.5 |
| 23.67 | 3.76 | 13.0 |
| 24.34 | 3.65 | 8.6 |

¹H-NMR showed no residual solvent (the stoichiometric ratio of Compound 1: acid was 1:0.5 FIG. 6H). TGA showed about 0.8% weight loss from about 34 to 129° C. (FIG. 6F). DSC showed no obvious melting peaks 32° C.), followed by an onset of 221° C. and a melting peak at about 235° C. (FIG. 6G). All the results showed that Compound 1 fumarate Form B was an anhydrate.

### Method B:

Compound 1 Form A (800 mg) and ACN (6.4mL) were added under stirring at 50° C. to obtain a mixture.

The mixture was slowly added with fumaric acid (92.71 mg, about 0.55 equivalent by molar ratio) and stirred for about 2 hours, and then added with Compound 1 fumarate Form B seeds (5 mg), cooled to RT, and stirred for about 1 day. The obtained solid was filtrated and dried, to obtain the Compound 1 fumarate Form B (710 mg) as an off-white solid.

Compound 1 fumarate From B was highly crystalline. DSC showed no melting peak before decomposition. TGA showed about 0.7% weight loss at about 150° C. ¹H-NMR showed that Compound 1: fumaric acid was 1:0.5 and no residual solvent was detected. KF shows it contains about 0.3% water by weight, equivalent to 0.1 water molecules.

### Example 8A: Preparation of Compound 1 HBr salt Form A,

About 50 mg of Compound 1 Form A and 1 equiv. of HBr were added into acetone or ACN (acetonitrile), stirred at 50° C. for 2 hours and then at 25° C. for at least 12 hours, and filtered to obtain the desired product.

The XRPD pattern was used to characterize the obtained product, which showed that the product was in a crystalline designated as Compound 1 HBr salt Form A, see FIG. 7A. The peaks and percent peak intensities obtained from the XRPD analysis are listed in Table 7A.

TABLE 7A
XRPD pattern of Compound 1 HBr salt Form A.

| Pos. [°2θ] | d-spacing [Å] | Rel. Int. [%] |
|---|---|---|
| 6.98 | 12.66 | 75.7 |
| 8.90 | 9.93 | 16.0 |
| 9.54 | 9.26 | 2.6 |
| 10.68 | 8.27 | 9.3 |
| 11.32 | 7.81 | 7.1 |
| 13.89 | 6.37 | 35.2 |
| 15.84 | 5.59 | 3.0 |
| 16.19 | 5.47 | 18.8 |
| 17.78 | 4.99 | 38.4 |
| 18.44 | 4.81 | 60.8 |
| 18.89 | 4.69 | 15.2 |
| 19.07 | 4.65 | 9.6 |
| 19.79 | 4.48 | 75.5 |
| 20.90 | 4.25 | 30.6 |
| 21.23 | 4.18 | 12.5 |
| 22.18 | 4.00 | 6.1 |
| 22.59 | 3.93 | 27.4 |
| 23.97 | 3.71 | 100.0 |
| 24.60 | 3.62 | 9.8 |

¹H-NMR showed 0.6% acetone by weight (the stoichiometric ratio of Compound 1: acid was 1:1.3, FIG. 7D). TGA showed about 1.8% weight loss about 33 to 129° C. (FIG. 7B). DSC showed no obvious melting peaks (FIG. 7C). All the results showed that Compound 1 HBr salt Form A was an anhydrate.

### Example 9A: Preparation of Compound 1 Mesylate Form A

Compound 1 Form A (50 mg) and 1 equivalent of MSA (methanesulfonic acid) were added into acetone, stirred at 50° C. for 2 hours, and then at 25° C. for at least 12 hours, and filtered to obtain the desired product.

The XRPD pattern was used to characterize the obtained product, which showed that the product was in a crystalline designated as Compound 1 mesylate Form A, see FIG. 8A. The peaks and percent peak intensities obtained from the XRPD analysis are listed in Table 8A.

TABLE 8A
XRPD pattern of Compound 1 mesylate Form A.

| Pos. [°2θ] | d-spacing [Å]2 | Rel. Int. [%] |
|---|---|---|
| 5.50 | 16.06 | 78.6 |
| 6.42 | 13.76 | 49.1 |
| 10.40 | 8.50 | 7.2 |
| 10.96 | 8.07 | 12.3 |
| 13.03 | 6.79 | 9.3 |
| 13.44 | 6.58 | 9.9 |
| 14.40 | 6.15 | 17.8 |
| 15.03 | 5.89 | 17.4 |
| 16.38 | 5.41 | 24.5 |
| 16.62 | 5.33 | 40.8 |
| 17.36 | 5.11 | 100.0 |
| 18.98 | 4.67 | 7.6 |
| 19.48 | 4.55 | 35.3 |
| 19.72 | 4.50 | 21.6 |
| 20.09 | 4.42 | 14.1 |
| 20.67 | 4.29 | 32.6 |
| 20.97 | 4.23 | 27.2 |
| 21.35 | 4.16 | 61.2 |
| 21.90 | 4.06 | 32.7 |
| 22.23 | 4.00 | 25.4 |
| 22.53 | 3.94 | 46.8 |
| 22.61 | 3.93 | 39.8 |
| 24.10 | 3.69 | 10.1 |

¹H-NMR showed 1.8% acetone by weight (0.20 equivalent by molar ratio, the stoichiometric ratio of Compound 1: acid was 1:1.1, FIG. 8D). TGA showed about 0.8% weight loss about 34 to 70° C. (FIG. 8B). DSC showed an endothermic peak at 66° C. (Tonset=31.4), followed by the onset of about 186° C. and a melting peak at about 194° C. (FIG. 8C), and then an exothermic peak with an onset of about 200° C.

### Example 9B: Preparation of Compound 1 Mesylate Form B

About 50 mg of Compound 1 Form A and 1 equiv. of MSA were added into ACN (acetonitrile), stirred at 50° C. for 2 hours and then at 25° C. for at least 12 hours, and filtered to obtain the desired product.

The XRPD pattern was used to characterize the obtained product, which showed that the product was in a crystalline designated as Compound 1 mesylate Form B, see FIG. 8E. The peaks and percent peak intensities obtained from the XRPD analysis are listed in Table 8B.

TABLE 8B
XRPD pattern of Compound 1 mesylate Form B.

| Pos. [°2θ] | d-spacing [Å] | Rel. Int. [%] |
|---|---|---|
| 5.57 | 15.86 | 100.0 |
| 5.95 | 14.85 | 26.7 |
| 6.55 | 13.48 | 18.5 |
| 6.99 | 12.64 | 11.1 |
| 7.88 | 11.21 | 2.0 |
| 10.03 | 8.82 | 5.9 |
| 11.05 | 8.00 | 9.6 |
| 14.65 | 6.04 | 18.6 |
| 15.48 | 5.72 | 10.9 |
| 15.95 | 5.55 | 10.3 |
| 16.59 | 5.34 | 24.6 |
| 17.43 | 5.08 | 15.6 |
| 18.39 | 4.82 | 32.3 |
| 19.26 | 4.60 | 6.1 |
| 20.01 | 4.43 | 14.0 |
| 20.52 | 4.33 | 33.7 |
| 20.88 | 4.25 | 20.2 |
| 21.55 | 4.12 | 19.5 |
| 22.26 | 3.99 | 29.1 |
| 22.87 | 3.88 | 13.7 |

¹H-NMR showed 0.6% ACN by weight (0.09 equivalent by molar ratio, the stoichiometric ratio of Compound 1: acid was 1:1.1, FIG. 8H). TGA showed about 2.7% weight loss about 31 to 124° C. (FIG. 8F). DSC showed an endothermic peak at 66° C. (Tonset=57° C.), followed by an exothermic peak with an onset of 215° C. (FIG. 8G).

### Example 9C: Preparation of Compound 1 Mesylate Form C

To a mixture of Compound 1 (20 g, 7.2 mmol) in 400 mL of acetone/H₂O (v/v=9/1) was added about 1.0 equivalent methane sulfonic acid (MSA) (3.47 g, 7.2 mmol) under stirring at 20° C., the solution was stirred at 20° C. for 5 hours, and filtered. The filter cake was washed with acetone and dried at 50° C. to obtain the desired product (18 g).

The XRPD pattern was used to characterize the obtained product, which showed that the product was in a crystalline designated as Compound 1 mesylate Form C, see FIG. 8I. The peaks and percent peak intensities obtained from the XRPD analysis are listed in Table 8C.

TABLE 8C
XRPD pattern of Compound 1 mesylate Form C.

| Pos. [°2θ] | d-spacing [Å]2 | Rel. Int. [%] |
|---|---|---|
| 5.55 | 15.90 | 4.3 |
| 6.65 | 13.29 | 100.0 |
| 7.73 | 11.43 | 18.3 |
| 10.78 | 8.20 | 4.4 |
| 11.79 | 7.50 | 2.4 |
| 12.21 | 7.24 | 2.3 |
| 12.77 | 6.93 | 31.3 |
| 13.25 | 6.68 | 33.6 |
| 14.70 | 6.02 | 2.9 |
| 15.44 | 5.74 | 25.1 |
| 15.76 | 5.62 | 3.3 |
| 16.31 | 5.43 | 18.1 |
| 16.62 | 5.33 | 31.3 |
| 16.90 | 5.24 | 8.8 |
| 19.29 | 4.60 | 6.7 |
| 19.54 | 4.54 | 7.0 |
| 19.91 | 4.46 | 10.8 |
| 20.90 | 4.25 | 3.9 |
| 21.59 | 4.11 | 53.2 |
| 21.84 | 4.07 | 37.1 |
| 22.65 | 3.92 | 4.1 |
| 22.96 | 3.87 | 15.6 |
| 23.21 | 3.83 | 19.5 |
| 23.85 | 3.73 | 2.0 |
| 24.43 | 3.64 | 3.6 |

¹H-NMR showed 0.4% acetone by weight (the stoichiometric ratio of Compound 1: acid was 1:1.1, FIG. 8L). TGA showed about 1.9% weight loss at about 80° C. and about 4.4% weight loss from 80° C. to 130° C. (FIG. 8J). DSC showed an endothermic peak at about 122° C. (Tonset =57° C.), followed by an exothermic peak (FIG. 8K). KF showed it contained about 6.3% water by weight, 2.4 equivalent by molar ratio.

### Example 10A: Preparation of Compound 1 Esilate Form A

Compound 1 Form A (50 mg) and 1 equiv. of ethanesulfonic acid were added into acetone, stirred at 50° C. for 2 hours and then at 25° C. for at least 12 hours, and filtered to obtain Compound 1 esilate Form A.

The XRPD pattern was used to characterize the obtained product, which showed that the product was in a crystalline designated as Compound 1 esilate Form A, see FIG. 9A. The peaks and percent peak intensities obtained from the XRPD analysis are listed in Table 9A.

TABLE 9A
XRPD pattern of Compound 1 Esilate Form A.

| Pos. [°2θ] | d-spacing [Å] | Rel. Int. [%] |
|---|---|---|
| 6.77 | 13.04 | 21.6 |
| 7.43 | 11.89 | 25.7 |
| 12.05 | 7.34 | 8.9 |
| 13.16 | 6.72 | 20.9 |
| 13.59 | 6.51 | 20.5 |
| 13.76 | 6.43 | 19.4 |
| 14.97 | 5.91 | 39.4 |
| 16.09 | 5.51 | 18.9 |
| 16.70 | 5.31 | 42.0 |
| 16.97 | 5.22 | 12.8 |
| 18.05 | 4.91 | 51.0 |
| 19.88 | 4.46 | 46.6 |
| 20.17 | 4.40 | 40.2 |
| 21.22 | 4.18 | 13.3 |
| 21.80 | 4.07 | 14.1 |
| 22.06 | 4.03 | 27.4 |
| 23.03 | 3.86 | 20.5 |
| 23.50 | 3.78 | 18.7 |
| 24.09 | 3.69 | 16.3 |
| 24.47 | 3.63 | 11.4 |
| 24.90 | 3.57 | 11.8 |

¹H-NMR showed 0.2% acetone by weight (0.03 equivalent by molar ratio, the stoichiometric ratio of Compound 1: acid was 1:1.1, FIG. 9D). TGA showed about 1.4% weight loss from about 35° C. to 230° C. (FIG. 9B). DSC showed an endothermic peak at about 257° C. (Tonset=257° C.) (FIG. 9C).

### Example 10B: Preparation of Compound 1 Esilate Form B

About 50 mg of Compound 1 Form A and 1 equivalent of ethanesulfonic acid were added into ACN (acetonitrile), stirred at 50° C. for 2 hours, and then at 25° C. for at least 12 hours, and filtered to obtain.

The XRPD pattern was used to characterize the obtained product, which showed that the product was in a crystalline designated as Compound 1 esilate Form B, see FIG. 9E. The peaks and percent peak intensities obtained from the XRPD analysis are listed in Table 9B.

TABLE 9B
XRPD pattern of Compound 1 Esilate Form B

| Pos. [°2θ] | d-spacing [Å] | Rel. Int. [%] |
|---|---|---|
| 5.53 | 15.98 | 100.0 |
| 5.99 | 14.73 | 67.3 |
| 6.76 | 13.07 | 28.1 |
| 7.43 | 11.90 | 3.6 |
| 8.04 | 10.99 | 2.4 |
| 9.06 | 9.75 | 4.7 |
| 10.25 | 8.62 | 8.7 |
| 10.97 | 8.06 | 12.2 |
| 12.67 | 6.98 | 4.1 |
| 13.78 | 6.42 | 12.0 |
| 14.43 | 6.13 | 18.3 |
| 15.97 | 5.55 | 12.3 |
| 16.43 | 5.39 | 32.0 |
| 16.84 | 5.26 | 10.8 |
| 17.30 | 5.12 | 16.0 |
| 17.86 | 4.96 | 4.5 |
| 18.31 | 4.84 | 19.4 |
| 18.69 | 4.74 | 21.8 |
| 19.17 | 4.63 | 21.1 |
| 19.42 | 4.57 | 19.2 |
| 20.30 | 4.37 | 13.3 |
| 20.56 | 4.32 | 38.2 |
| 21.95 | 4.05 | 8.4 |
| 22.19 | 4.00 | 17.0 |
| 22.44 | 3.96 | 19.7 |
| 23.02 | 3.86 | 16.4 |
| 24.03 | 3.70 | 10.5 |

¹H-NMR showed 0.4% ACN by weight (0.07 equivalent by molar ratio, the stoichiometric ratio of Compound 1: acid was 1:1.0, FIG. 9H). TGA showed about 2.6% weight loss from 34° C. to 100° C., and 0.7% weight loss from 100° C. to 161° C. (FIG. 9F). DSC showed an endothermic peak at 87° C. (Tonset=34° C.), followed by an onset of 133° C. and melting peak at 141° C., and an exothermic peak with an onset of 178.9° C. (FIG. 9G).

### Example 11: Evaluation of Compound 1 Salts in Different Forms

### 1) Bulk Stability

Bulk stability of Compound 1 mesylate Form C, phosphate Form A, maleate Form A, fumarate Form B was investigated under 25° C./60% RH in an open container and under 40° C./75% RH in an open container over 1 week. Bulk stability of Compound 1 hydrochloride Form C and Form D, were evaluated under 25° C./60% RH in an open container, under 40° C./75% RH in an open container, and at 60° C. in a tight container over 1 week. All the physical Forms were physically and chemically stable under these conditions.

In addition, some Forms were physically and chemically stable under 25° C./60% RH, 30° C./65% RH, and 40° C./75% RH in a tight container over six months.

### 2) Solubility

Solubility of the Compound 1 mesylate Form C, phosphate Form A, maleate Form A, and fumarate Form B was measured in 6 aqueous pH buffers and bio-relevant solutions including pH 1.2 HCl solution (0.1 N), pH 4.5 acetate buffer (50 mM), water, pH 2.0 SGF, pH 6.5 FaSSIF-v1, and pH 5.0 FeSSIF-v1 at 37° C. for 1 h and 2 h. Residual solids after the solubility test were analyzed by XRPD.

All four salts showed a pH-dependent solubility profile. The four salts showed relatively high solubility (0.1-0.7 mg/mL in free Form) in pH 1.2 buffer but dropped to about 0.001 mg/mL in pH 4.5 buffer. In SGF, the phosphate Form A showed the highest solubility of about 0.74 mg/mL at 1 h. In FeSSIF-v1 and FaSSIF-v1, all four salts showed similar solubility to that in pH 4.5 buffer. After the solubility test, residual solids changed to potential hydrochloride in pH 1.2 buffer and SGF media or to free Form in the other media except for fumarate salt. The fumarate salt remained unchanged in SGF.

### 3) Hygroscopicity

Hygroscopicity of Compound 1 mesylate Form C, phosphate Form A, maleate Form A, fumarate Form B, hydrochloride Form C, and hydrochloride Form D were evaluated by dynamic vapor sorption (DVS) test at 25° C.
Compound 1 mesylate Form C is slightly hygroscopic. It absorbed about 1.1% water from 40% RH to 95% RH at 25° C. No Form and crystallinity changed after the DVS test.
Compound 1 phosphate Form A is slightly hygroscopic. It absorbed about 2.3% water from 40% RH to 95% RH at 25° C. No Form and crystallinity change after the DVS test.
Compound 1 maleate Form A is slightly hygroscopic. It absorbed about 0.4% water from 40% RH to 95% RH at 25° C. No Form and crystallinity changed after the DVS test.
Compound 1 fumarate Form B was stable from 0% RH to 40% RH. However, it started to absorb water when relative humidity was above 40% and converted to a potential hydrate. The potential hydrate underwent dehydration when RH<70% and returns back to the anhydrate. After the DVS test, the obtained sample was still Compound 1 fumarate Form B.
Compound 1 hydrochloride Form C is slightly hygroscopic. It absorbed about 0.29% water from 40% RH to 95% RH at 25° C. No Form changed after the DVS test.
Compound 1 hydrochloride Form D is non-hygroscopic. It absorbed about 0.14% water from 40% RH to 95% RH at 25° C. After the DVS test. No Form changed after the DVS test.

### 4) Feasibility of Formulation Process

Feasibility of Formulation process for Compound 1 mesylate Form C, phosphate Form A, maleate Form A, fumarate Form B hydrochloride Form C, and hydrochloride Form D were evaluated by compression (at 2 MPa, 5 MPa, 10 MPa, and 20 MPa), dry grinding, and granulation simulation experiments.

All the salts showed good tolerance to compression, dry grinding, granulation simulation with no Form change and obvious crystallinity decrease, except Compound 1 fumarate Form B. After dry grinding and compression, Compound 1 fumarate Form B showed Form change. However, the peaks of Compound 1 hydrochloride Form D became slightly broad after compression under 5 MPa and 10 MPa.

Compound 1 hydrochloride Form C, showed good tolerance to compression with no Form change and no obvious crystallinity decrease, but the peaks became slightly broad when compression at 5 MPa, 10 MPa, and 20 MPa.

While the invention has been described in connection with specific embodiments thereof, it will be understood that it is capable of further modifications, and this application is intended to cover any variations. uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A crystalline Form of N-(5-(2,2-dimethyl-2,3-dihydro-[1,4]dioxino[2,3-b]pyridin-6-yl)-4-((6-(methylsulfonyl)-4-(tetrahydro-2H-pyran-4-yl)pyridin-2-yl)amino)pyridin-2-yl)acetamide or a pharmaceutically acceptable salt thereof.

2. (canceled)

3. The crystalline form according to claim 1, wherein the crystalline Form has an X-ray powder diffraction pattern comprising diffraction peaks having °2θ angle values at 10.3±0.2°, 14.8±0.2°, and 16.2°±0.2°.

4. The crystalline form according to claim 1, wherein the crystalline Form has an X-ray powder diffraction pattern comprising diffraction peaks having °2θ angle values at 10.3±0.2°, 14.8±0.2°, 16.2±0.2°, 20.7±0.2° and 21.6±0.2°.

5. The crystalline form according to claim 1, wherein the crystalline Form has an X-ray powder diffraction pattern comprising diffraction peaks having °2θ angle values at 7.5±0.2°, 10.3±0.2°, 14.8±0.2°, 16.2±0.2°, 20.7±0.2°, and 21.6±0.2°

6. The crystalline form according to claim 1, wherein the crystalline Form has an X-ray powder diffraction pattern comprising diffraction peaks having °2θ angle values at 7.5±0.2°, 10.3±0.2°, 14.8±0.2°, 15.4±0.2°, 16.2±0.2°, 19.8±0.2°, 20.7±0.2°, 21.6±0.2°, 22.8±0.2° and 25.0±0.2°.

7. The crystalline form according to claim 1, wherein the crystalline form has an XRPD pattern substantially as shown in FIG. 1A.

8. The crystalline form according to claim 1, wherein the crystalline form is characterized by having one endotherm peak at 305±5° C. by differential scanning calorimetry (DSC).

9. The crystalline form according to claim 1, wherein the crystalline form has a DSC thermogram substantially as shown in FIG. 1B.

10-95. (canceled)

96. A pharmaceutical composition, comprising a therapeutically effective amount of a crystalline form according to claim 1, and at least one pharmaceutically acceptable excipient.

97. A method for treating an inflammatory or autoimmune disease in a subject in need thereof comprising administering to the subject with a therapeutically effective amount of a crystalline form of claim 1.

98. The method according to claim 97, wherein the subject is a human.

99-108. (canceled)
